(19) United States
(12) Patent Application Publication
(10) Pub. No.: US 2021/0270847 A1
(43) Pub. Date: Sep. 2, 2021

(54) PROTEIN AND PEPTIDE BIOMARKERS FOR TRAUMATIC INJURY TO THE CENTRAL NERVOUS SYSTEM

(71) Applicant: UNIVERSITY OF FLORIDA RESEARCH FOUNDATION, INCORPORATED, Gainesville, FL (US)

(72) Inventors: Kevin Ka-Wang WANG, Gainesville, FL (US); George Anis SARKIS, Lexington, MA (US); Manasi KAMAT, Gainesville, FL (US); Hamad YADIKAR, Gainesville, FL (US); Ahmed MOGHIEB, Royersford, PA (US)

(21) Appl. No.: 17/269,041

(22) PCT Filed: Aug. 19, 2019
(86) PCT No.: PCT/US2019/047030

§ 371 Date: Feb. 17, 2021

Related U.S. Application Data
(60) Provisional application No. 62/719,254, filed on Aug. 17, 2018.

(51) Int. Cl. G01N 33/68

(52) U.S. Cl. CPC G01N 33/6896; G01N 2800/28; G01N 2496/00

(57) ABSTRACT

The invention relies on detection of specific identified proteins, protein breakdown products, and peptide fragments, to diagnose and evaluate traumatic brain injury, spinal cord injury, and any traumatic injury to the CNS in a subject. These analytes (proteins, protein breakdown products thereof, and peptide fragments thereof) are released from injured tissue into blood and/or cerebrospinal fluid, and can be used to identify the central nervous system cell types (i.e. neuron, astrocyte, oligodendrocyte, and the like) or subcellular structure (e.g., axon, dendrites, presynaptic terminal, post-synaptic terminal, and extracellular matrix) affected, and to determine the diagnosis, location, and severity of the injury. Time course measurements of these analytes measured at different times after an injury or suspected injury also are used as tools for diagnosis and prognosis of central nervous system injury. Proteins, protein breakdown products, and peptide fragments are claimed, as well as kits and methods for their use.

### STATEMENT OF GOVERNMENT INTEREST

This invention was made with Government support under Contract No. R21 NS085455-01 awarded by the National Institutes of Health. The government has certain rights in the invention.

### BACKGROUND

### Field of the Invention

The invention relates generally to protein and higher molecular weight protein breakdown products (ranging from about 85% or less of the size of the intact proteins to greater than 10 kDa) and lower molecular weight peptide fragment (ranging from 500 Da to 10, kDa) biomarkers that are released into biological fluids and can be measured in fluid biological samples, such as cerebrospinal fluid, blood, dialysate, or central nervous system tissue lysate, after traumatic injury to the central nervous system. Specifically, particular discrete anatomical regions of the brain, cell types, subcellular structures, and brain extracellular matrix can be identified as damaged through detection of these markers. The invention therefore also encompasses methods of diagnosis, prognosis and management of central nervous system injury.

### Background of the Invention

Injury to the central nervous system (CNS) occurs in a variety of medical conditions and in trauma, and has been the subject of intense scientific scrutiny in recent years. The brain has such high metabolic requirements that it can suffer permanent neurological damage if deprived of sufficient oxygen (hypoxia) for even a few minutes. Under conditions of hypoxia or anoxia, when mitochondrial production of ATP cannot meet the metabolic requirements of the brain, tissue damage occurs.

This process is exacerbated by neuronal release of the neurotransmitter glutamate, which stimulates NMDA (N-methyl-D-aspartate), AMPA (α-amino-3-hydroxy-5-methyl-4-isoxazole propionate) and kainate receptors. Activation of these receptors initiates calcium influx into the neurons and production of reactive oxygen species, which are potent toxins that damage important cellular structures such as membranes, DNA and enzymes.

The brain has many redundant blood supplies, which means that its tissue is seldom completely deprived of oxygen, even during acute ischemic events caused by thromboembolic events or trauma. A combination of the injury of hypoxia with the added insult of glutamate toxicity therefore is believed to be ultimately responsible for cellular death, therefore, if glutamate toxicity can be alleviated, neurological damage could also be lessened. Antioxidants and anti-inflammatory agents have been proposed to reduce damage, but they often have poor access to structures such as the brain, which is protected by the blood brain barrier.

Brain injury, such as cerebral apoplexy, is a result of a sudden circulatory disorder of a human brain area with subsequent functional losses and corresponding neurological and/or psychological symptoms. Cerebral apoplexy can be caused by cerebral hemorrhages (e.g., after a vascular tear in hypertension, arteriosclerosis and apoplectic aneurysms) and ischemia (e.g., due to a blood pressure drop crisis or embolism), leading to degeneration or destruction of the brain cells. After a cerebral vascular occlusion, only part of the tissue volume is destroyed as a direct result of the restricted circulation and the associated decreased oxygen supply. The tissue area designated as the infarct core can only be kept from dying off by immediate re-canalization of the vascular closure, e.g., by local thrombolysis, and is therefore only accessible to therapy in a very limited fashion. The outer peripheral zone, referred to as the penumbra, loses its function immediately after onset of the vascular occlusion, but initially remains adequately supplied with oxygen by the collateral supply and becomes irreversibly damaged after only a few hours or days. Since the cell death in this area does not occur immediately, methods to block the damage after stroke and trauma have been investigated. However, without early diagnosis, the prognosis for such subjects is poor.

The mammalian nervous system comprises the peripheral nervous system (PNS) and the central nervous system (CNS, comprising the brain and spinal cord), and is composed of two principal classes of cells: neurons and glial cells. The glial cells fill the spaces between neurons, nourishing them and modulating their function. Certain glial cells, such as Schwann cells in the PNS and oligodendrocytes in the CNS, also provide a protective myelin sheath that surrounds and protects neuronal axons, the processes that extend from the neuron cell body and through which the electric impulses of the neuron are transported. In the peripheral nervous system, the long axons of multiple neurons are bundled together to form a nerve or nerve fiber. These in turn may be combined into fascicles, such that the nerve fibers form bundles embedded together with the intraneural vascular supply in a loose collagenous matrix bounded by a protective multilamellar sheath. In the central nervous system, the neuron cell bodies are visually distinguishable from their myelin-sheath processes, giving rise to the terms gray matter, referring to the neuron cell bodies, and white matter, referring to the myelin-covered processes.

During development, differentiating neurons from the central and peripheral nervous systems send out axons that must grow and make contact with specific target cells. In some cases, growing axons must cover enormous distances; some extend into the periphery, whereas others stay confined within the central nervous system. In mammals, this stage of neurogenesis is complete during the embryonic phase of life and neuronal cells do not multiply once they have fully differentiated. Accordingly, the neural pathways of a mammal are particularly at risk if neurons are subjected to mechanical or chemical trauma or neuropathic degeneration sufficient to put the neurons that define the pathway at risk of dying.

A host of neuropathies, some of which affect only a subpopulation or a system of neurons in the peripheral or central nervous systems, have been identified to date. The neuropathies, which may affect the neurons themselves or the associated glial cells, may result from cellular metabolic dysfunction, infection, exposure to toxic agents, autoimmunity dysfunction, malnutrition or ischemia. In some cases the cellular dysfunction is thought to induce cell death directly. In other cases, the neuropathy may induce sufficient tissue necrosis to stimulate the body's immune/inflammatory system and the body's immune response to the initial neural injury then destroys the neurons and the pathway defined by these neurons.

Another common injury to the CNS is stroke, the destruction of brain tissue as a result of intracerebral hemorrhage or infarction. Stroke is a leading cause of death in the developed world. Injury after stroke can be caused by reduced blood flow (ischemia or ischemic stroke) that results in deficient blood supply and death of tissues in one area of the brain (infarction). Causes of ischemic strokes include blood clots that form in the blood vessels in the brain (thrombi) and blood clots or pieces of atherosclerotic plaque or other material that travel to the brain from another location (emboli). Bleeding (hemorrhage) within the brain may also cause symptoms that mimic ischemic stroke.

Mammalian neural pathways also are at risk due to damage caused by neoplastic lesions. Neoplasias of both the neurons and glial cells have been identified. Transformed cells of neural origin generally lose their ability to behave as normal differentiated cells and can destroy neural pathways by loss of function. In addition, the proliferating tumors may induce lesions by distorting normal nerve tissue structure, inhibiting pathways by compressing nerves, inhibiting cerebrospinal fluid or blood supply flow, and/or by stimulating the body's immune response. Metastatic tumors, which are a significant cause of neoplastic lesions in the brain and spinal cord, may similarly damage neural pathways and induce neuronal cell death.

In 2010, about 2.5 million emergency department visits, hospitalizations or deaths were associated with traumatic brain injury (TBI), either alone or in combination with other injuries, in the United States. TBI contributed to the deaths of more than 50,000 people and was diagnosed in more than 280,000 hospitalizations.

Over the past decade (2001-2010), while rates of TBI-related emergency visits increased by 70%, hospitalization rates increased by only 11% and death rates decreased by 7%. In 2009, an estimated 248,418 children ages 20 or younger were treated for TBI in the United States. Emergency room visits for sports and recreation-related injuries included a diagnosis of concussion or TBI. From 2001 to 2009 the rate of emergency room visits for sports and recreation-related injuries with a diagnosis of concussion or TBI, alone or in combination with other injuries, rose 57% among children and young adults.

Chronic Traumatic Encephalopathy (CTE) is a progressive degenerative disease resulting from repetitive TBI. This type of injury was previously called punch-drunk syndrome or dementia pugilistica. CTE is commonly found in professional athletes participating in contact sports such as boxing, rugby, American football, ice hockey, and professional wrestling. It has also been found in soldiers exposed to blast or concussive injury. Symptoms associated with CTE may include dementia such as memory loss, aggression, confusion and depression, which generally appear years or decades after the trauma.

It has been hypothesized that the pathological process that leads to acute traumatic injury to the CNS consists of two steps. The primary injury results from the physical and mechanical force or blast overpressure wave as a result of direct impact to the CNS tissue. The secondary injury is the cascade of biochemical events such as proteolysis of cytoskeletal, membrane, and myelin proteins due to the elevation in intracellular Ca²⁺ that activates cysteine proteases such as calpain. The proteolysis causes progressive tissue degeneration, including neuronal cell death, axonal degeneration, and demyelination.

Neurological examinations are currently used for diagnosis, determination of severity, and prediction of neurological outcome in the brain injuries such as TBI and stroke. Although these tests can diagnose acute brain injury, assessment of injury severity and prognosis is often challenging. Current methods often cannot accurately assess the severity of TBI or predict long-term outcomes of TBI subjects. It also has been difficult to pinpoint the exact area of the brain or the cell type that has been injured. In addition, the neurological and functional recovery of TBI subjects is highly variable.

Therefore, there is a need in the art, not only for improved methods to diagnose traumatic injury to tissues of the central and peripheral nervous system, but also for new methods that can more discretely identify the nature and the extent of the injury for purposes of diagnosis and prognosis, and to guide treatment protocols.

### SUMMARY OF THE INVENTION

Diagnostic clinical assessments of nervous system injury severity and therapeutic treatment efficacy have been studied, including biomarkers that can indicate brain damage and traumatic brain injury. The discovery and use of biomarkers for TBI is expected to lead to development of new therapeutic interventions that can be applied to prevent or reduce disability due to TBI. Biomarkers generated after brain damage have not been associated with specific regions or cell types, however. Identification of neurochemical markers specific to or predominantly found in the nervous system (CNS and PNS) would prove immensely beneficial for both prediction of outcome and guidance of targeted therapeutic delivery.

Therefore, the invention relates to a method of diagnosing trauma to the central nervous system in a subject in need thereof, comprising testing a first fluid biological sample obtained from the subject for the level of at least two proteins, protein breakdown products, or peptide fragments of one or more proteins selected from the group consisting of (a) Synapsin (Synapsin I, Synapsin II, Synapsin III); (b) Glutamate decarboxylase (GAD1; GAD2); (c) Golli-Myelin Basic Protein 1; (d) Golli-Myelin Basic Protein 1 in combination with classic Myelin Basic Protein Isoform 5; (e) Microtubule associated protein 6 (MAP6); (f) Neurogranin; (g) Vimentin; (h) Vimentin in combination with Glial Fibrillary Acidic Protein; (i) Tau-758 isoform; (j) Tau-758 isoform in combination with Tau-441 isoform; (k) Glial fibrillary acidic protein (GFAP); (1) Cortexin (Cortexin 1, Cortexin 2, Cortexin 3); (m) Striatin; (n) Neurexin (Neurexin-1, Neurexin-2, Neurexin-3); (o) Brain acidic soluble protein 1 (BASP1); (p) GAP43; (q) Calmodulin Regulated Spectrin Associated Protein (CAMSAP1, CAMSAP2, CAMSAP3); (r) Chondroitin Sulfate Proteoglycan 4; (s) Neurocan; and (t) Brevican; wherein levels of the at least two proteins or protein breakdown products that are at least two-fold higher in the fluid biological sample from the subject than the levels of the at least two proteins or protein breakdown products in a fluid biological sample from an uninjured subject indicate the presence of a central nervous system injury. In addition, the invention relates to a method of diagnosing trauma to the central nervous system in a subject in need thereof, comprising testing a first fluid biological sample obtained from the subject for the level of at least two proteins, protein breakdown products, or peptide fragments of one or more proteins selected from the group consisting of (a) Synapsin (Synapsin I, Synapsin II, Synapsin III); (b) Tau-441 isoform; (c) Tau-758 isoform; (d) Neurogranin; (e) Vimentin; (f) Classic Myelin Basic Protein Isoform 5; (g) Golli-Myelin Basic Protein 1; (h) Glial Fibrillary Acidic Protein; and (i) MAP6, (j) complement protein Clq (Clqa, Clqb, Clqc), C3, C5, C1s, C1QRF and complement receptor CR1; wherein levels of the at least two peptide fragments that are at least two-fold higher in the fluid biological sample from the subject than the levels of the at least two peptide fragments in a fluid biological sample from an uninjured subject indicate the presence of a central nervous system injury.

In preferred embodiments, the at least two peptide fragments are selected from the group consisting of:

Tau-441 peptides:
(SEQ ID NO: 471)
AEPRQEFEVMEDHAGTYGLG;
(SEQ ID NO: 472)
AAQPHTEIPEGTTAEEALEDEAAGHVTQARMVS;
(SEQ ID NO: 473)
LSKVTSKCGSLG;
(SEQ ID NO: 474)
SPQLATLADEVSASLAK;
(SEQ ID NO: 475)
TLADEVSASLAKQGL;
Tatt-758 (Tau-G) peptides:
(SEQ ID NO: 476)
PQLKARMVSKSKDGTGSDDKKAKTSTRSSA;
(SEQ ID NO: 477)
SPKHPTPGSSDPLIQPSSPAVCPEPPSSPKYVSSVTSRTGSSGAKEM;
(SEQ ID NO: 478)
PPSSPKYVSSVTSRTGSSGAKEMKLKGADGKTKIATPRGAA;
(SEQ ID NO: 479)
SVTSRTGSSGAKEMKLKGADGK;
(SEQ ID NO: 480)
SPKHPTPGSSDPLIQPSSPAVCPE;
(SEQ ID NO: 481)
PPSSPKYVSSVTSRTGSSGAKEMKL;
Neurogranin peptides:
(SEQ ID NO: 482)
ILDIPLDDPGANAAAAKIQAS(p)FRGHMARKKIKSGERGRKGPGPGGPG
GA;
(SEQ ID NO: 483)
ILDIPLDDPGANAAAAKIQASFRGHMARKKIKSGERGRKGPGPGGPGGA;
(SEQ ID NO: 484)
DDDILDIPLDDPGANAAAAKIQAS(p)FR;
(SEQ ID NO: 485)
DDDILDIPLDDPGANAAAAKIQASFR;
(SEQ ID NO: 486)
PGANAAAAKIQAS(p)FRGHMARKKIKSGERGRKGPGPGG;
(SEQ ID NO: 487)
PGANAAAAKIQASFRGHMARKKIKSGERGRKGPGPGG;
Vimentin peptides:
(SEQ ID NO: 488)
NVKMALDIEIAT;
(SEQ ID NO: 489)
LLEGEESRISLPLPNFSSLNLR;
(SEQ ID NO: 490)
NVKMALDIEIATYRKLLEGEESRISLPLPNFSSLNLRETNLDSLPLVDTH
SKR;
(SEQ ID NO: 491)
TLLIKTVETRDGQVIN;
(SEQ ID NO: 492)
MSTRSVSSSSYRRMFGGPGTASRPSSSRSYVTTSTRTYSLGSALRPSTSR
SLYASSPGGVYATRSSAVRLRSSVP
(SEQ ID NO: 493)
STRSVSSSSYRRMFGGPGTASRPSSSRSYVTTSTRTYSLGSALR;
MBP peptides:
(SEQ ID NO: 494)
HGSKYLATASTMD;
(SEQ ID NO: 495)
HGSKYLATASTMDHARHGFLPRHRDTGILDSIGR;
(SEQ ID NO: 496)
GRTQDENPVVHFFKNIVTPRTPPPSQGKGRGLSLSRF;
(SEQ ID NO: 497)
HKGFKGVDAQGTLS;
Golli-MBP1 isoform peptides:
(SEQ ID NO: 498)
HAGKRELNAEKASTNSETNRGESEKKRNLGELSRTT;
(SEQ ID NO: 499)
NAWQDAHPADPGSRPHLIRLFSRDAPGREDNTFKDRPSESDE;
GFAP peptides:
(SEQ ID NO: 500)
ITSAARRSYVSSGEMMVGGLAPGRRLGPGTRLSLARMP;
(SEQ ID NO: 501)
YVSSGEMMVGGLAPGRRLGPGTRLS;
(SEQ ID NO: 502)
RSYVSSGEMMVGGLAPGRRLGP;
(SEQ ID NO: 503)
AARRSYVSSGEMMVGGLAPGRRLGPGTRLSLARMPPPLPTR;
(SEQ ID NO: 504)
GEENRITIPVQTFSNLQIRETSLDTKSV;
(SEQ ID NO: 505)
QTFSNLQIRETSLDTKSVSEGHLKRNIVVKTVEMR;
(SEQ ID NO: 506)
DGEVIKES;
(SEQ ID NO: 507)
DGEVIKE;
(SEQ ID NO: 508)
DGEVIKESKQEHKDVM;
(SEQ ID NO: 509)
TKYSEATEHPGAPPQPPPPQQ;
(SEQ ID NO: 510)
QLPTVSPLPRVMIPTAPHTEYIESS.
Complement C1q subcomponent subunit B
(D6R934) peptide:
(SEQ ID NO: 701)
HGEFGEKGDPGIPG;
Complement C3 (P01024) peptide:
(SEQ ID NO: 702)
HWESASLL;
(SEQ ID NO: 703)
VKVFSLAVNLIAI;
Complement C5 (P01031) peptide:
(SEQ ID NO: 705)
VTcTNAELVKGRQ;
Complement C1s (P09871) peptide:
(SEQ ID NO: 706)
IISGDTEEGRLCGQRSSNNPHSPIVE;
Complement receptor type 1 CR1 (E9PDY4)
peptides:
(SEQ ID NO: 704a)
KTPEQFPFAS;
(SEQ ID NO: 704b)
SCDDFMGQLLNGRVLFPVNLQLGAK;
Microtubule-associated Protein 6 (MAP6)
(Q7TSJ2) peptides:
(SEQ ID NO: 179)
TKYSEATEHPGAPPQPPPPQQ;
(SEQ ID NO: 180)
QLPTVSPLPRVMIPTAPHTEYIESS;
Synapsin I (SYNI) (P17600-1 or P17600-2)
peptides:
(SEQ ID NO: 181)
QDEVKAETIRS;
Synapsin II (SYN2) (Q9277-1 or Q64332)
peptides:
(SEQ ID NO: 182)
SQSLTNAFSFSESSFFRS;
Synapsin III (SYN3) (Q14994-1 or P07437)
peptides:
(SEQ ID NO: 183)
DWSKYFHGKKVNGEIEIRV;
and
((SEQ ID NO: 184)
GEHVEEDRQLMADLVVS

Also, in preferred embodiments, the first fluid biological sample is obtained from the subject within 24 hours of the trauma to the central nervous system or within 3 days of the trauma to the central nervous system. In other embodiments, the one or more additional fluid biological samples are obtained from the subject at subsequent times to the first fluid biological sample.

Preferably, the testing comprises subjecting the fluid biological samples are subjected to ultrafiltration using a ultrafiltration membrane filter with a molecular weight cutoff of about 10,000 Da to separate an ultrafiltrate fraction and then subjecting the ultrafiltrate fraction to assay for proteins, protein breakdown products or peptide fragments. In certain embodiments, an increasing level of the at least two proteins, protein breakdown products, or peptide fragments in fluid biological samples taken at subsequent times indicates worsening of the severity of the central nervous system injury; a decreasing level of the at least two proteins, protein breakdown products, or peptide fragments in fluid biological samples taken at subsequent times indicates improvement in the central nervous system injury; and an unchanging level of the at least two proteins, protein breakdown products, or peptide fragments in fluid biological samples taken at subsequent times indicates a leveling of the severity of the central nervous system injury.

Embodiments of the invention also include a method of identifying the anatomical location of trauma to the central nervous system in a subject in need thereof, comprising testing a fluid biological sample obtained from the subject for the presence of any combination of (a) one or more cortexin proteins, protein breakdown products, or peptide fragments, the presence of which above control levels identifies the cortex as the anatomical location; (b) one or more myelin basic protein proteins, protein breakdown products, or peptide fragments, the presence of which above control levels identifies the white matter as the anatomical location; and (c) one or more striatin proteins, protein breakdown products, or peptide fragments, the presence of which above control levels identifies the striatum as the anatomical location.

Further embodiments of the invention include a method of identifying cell types injured in trauma to the central nervous system in a subject in need thereof, comprising testing a fluid biological sample obtained from the subject for the presence of any combination of (a) one or more protein, or protein breakdown product of brain acidic soluble protein−1, glutamate decarboxylase 1, glutamate decarboxylase 2, neurochondrin or any combination thereof, the presence of which above control levels identifies the cell type as neurons; (b) one or more protein, or protein breakdown product of Vimentin, the presence of which above control levels identifies the cell type as astroglia; and (c) one or more protein, or protein breakdown product of myelin basic protein 5 or Golli-myelin basic protein, the presence of which above control levels identifies the cell type as oligodendrocytes and complent protein Clq (Clqa, Clqb, Clqc), C3, C5, C1s, Clq ligand and complment receptor CR1 from microglia cells. Additional embodiments include a method of identifying the subcellular location of injury to the central nervous system after trauma in a subject in need thereof, comprising testing a fluid biological sample obtained from the subject for the presence of any combination of (a) one or more protein, or protein breakdown product of neurexin-1, neurexin-2, neurexin-3, synapsin-I, synapsin-II, synapsin-III or any combination thereof, the presence of which above control levels identifies the subcellular location as the presynaptic terminal; (b) one or more protein, or protein breakdown product of neurogranin, the presence of which above control levels identifies the subcellular location as the post-synaptic terminal; (c) one or more protein, or protein breakdown product of brain acidic soluble protein 2, growth associated protein 43 or a combination thereof, the presence of which above control levels identifies the subcellular location as the growth cone; (d) one or more protein, or protein breakdown product of nesprin-1, the presence of which above control levels identifies the subcellular location as the neuronal nucleus; (e) one or more protein, or protein breakdown product of Calmodulin regulated spectrin-associated protein 1, Calmodulin regulated spectrin-associated protein 2, Calmodulin regulated spectrin-associated protein 3, or any combination thereof, the presence of which above control levels identifies the subcellular location as the cortical cytoskeleton and axon; (f) one or more protein, or protein breakdown product of microtubule associated protein 6, the presence of which above control levels identifies the subcellular location as dendrites; and (g) one or more protein, or protein breakdown product of chondroitin sulfate proteoglycan 4, neurocan, brevican or any combination thereof, the presence of which above control levels identifies the subcellular location as the extracellular matrix.

The invention also includes embodiments such as a method of diagnosing the severity of trauma to the central nervous system in a subject in need thereof, comprising the steps of (a) testing a first fluid biological sample obtained from the subject up to 3 days after central nervous system injury for the levels of one or more proteins, protein breakdown products, and peptide fragments derived from a protein selected from one or more of Synapsin I, Synapsin II, Synapsin III, Tau-441 isoform, Tau-758 isoform, neurogranin, Vimentin, myelin basic protein Isoform 5, Golli-myelin basic protein 1, complement protein Clq (Clqa, Clqb, Clqc), C3, C5, Cls, Clq ligand and complment receptor CR1 and glial fibrillary acidic protein; (b) testing a second subsequent fluid biological sample obtained from the subject subsequent to the first fluid biological sample for the levels of the same one or more proteins, protein breakdown products, and peptide fragments as step (a); (c) optionally testing further subsequent fluid biological samples for the levels of the same one or more proteins, protein breakdown products, and peptide fragments as step (a); (d) comparing the levels of the one or more proteins, protein breakdown products, and peptide fragments in the fluid biological samples to a control sample from an uninjured subject and to each other; and (e) when the levels of peptide breakdown products in the fluid biological samples increase in subsequent samples, diagnosing a severe central nervous system injury.

Embodiments of the invention include a method of distinguishing severe trauma to the central nervous system with pathoanatomical lesions detectable by CT, MRI, or both, from less severe central nervous system trauma with no detectable pathoanatomical lesions in a subject in need thereof, comprising (a) testing at least one first fluid biological sample obtained from the subject within 24 hours after central nervous system injury for the levels of one or more peptide fragments of a protein selected from one or more of Synapsin I, Synapsin II, Synapsin III, Tau-441 isoform, Tau-758 isoform, neurogranin, Vimentin, myelin basic protein isoform 5, Golli-myelin basic protein 1, a complement protein and glial fibrillary acidic protein; (b) testing a second subsequent fluid biological sample obtained from the subject about 2 days to about 6 months subsequent to the first fluid biological sample for the levels of the same one or more peptide fragments as step (a); (c) comparing the levels of the same one or more peptide fragments in the first and second fluid biological samples to a control sample from an uninjured subject and to each other; and (d) when the levels of the same one or more peptide fragments in the first fluid biological sample are above those in the control sample but decrease in the second fluid biological samples, diagnosing an acute central nervous system injury; and when the levels of the same one or more peptide fragments in the first fluid biological samples are above those in the control sample and increase or remain constant in subsequent samples, diagnosing a chronic central nervous system injury.

Embodiments of the invention also include a method of determining the damaged central nervous system anatomical areas, cell types and subcellular structures in a subject with central nervous system injury in need thereof, comprising (a) testing a fluid biological sample obtained from the subject after central nervous system injury for the levels of one or more proteins, protein breakdown products and/or peptide fragments of (1) a protein selected from cortexin-1, cortexin-2, cortexin-3 and any combination thereof; (2) a protein selected from myelin basic protein 5, Golli-myelin basic protein and a combination thereof; and (3) the protein striatin; (b) testing the fluid biological sample for the levels of one or more proteins, protein breakdown products and/or peptide fragments of (1) a protein selected from brain acidic soluble protein 1, glutamine decarboxylase 1, glutamate decarboxylase 2, neurochondrin or any combination thereof; (2) Vimentin; and (3) a protein selected from myelin basic protein 5, Golli-myelin basic protein and a combination thereof; and (c) testing the fluid biological sample for the levels of one or more proteins, protein breakdown products and/or peptide fragments of (1) a protein selected from cortexin-1, cortexin-2, cortexin-3, neurexin-1, neurexin-2, neurexin-3 and any combination thereof; (2) neurogranin; (3) BASP2/GAP43; (4) nesprin-1; (5) a protein selected from calmodulin regulated spectrin-associated protein 1, calmodulin regulated spectrin-associated protein 2, calmodulin regulated spectrin-associated protein 3, Tau-441, Tau-758 and any combination thereof; (6) microtubule associated protein 6; and (7) a protein selected from chondroitin sulfate proteoglycan 4, neurocan, brevican, or any combination thereof; wherein the presence of levels above control of cortexin-1, cortexin-2, or cortexin-3 is associated with cerebrocortical injury; the presence of levels above control of myelin basic protein 5 or Golli-myelin basic protein is associated with white matter or myelin sheath injury; the presence of levels above control of striatin is associated with striatum injury; the presence of levels above control of brain acidic soluble protein 1, glutamine decarboxylasel, glutamine decarboxylase 2 or neurochondrin is associated with neuronal cell body injury; the presence of levels above control of Vimentin is associated with astroglial injury; the presence of levels above control of myelin basic protein 5 or Golli-myelin basic protein is associated with oligodendrocyte injury; the presence of levels above control of cortexin-1, cortexin-2, cortexin-3, neurexin-1, neurexin-2, or neurexin-3 is associated with presynaptic terminal damage; the presence of levels above control of neurogranin is associated with post-synaptic terminal damage; the presence of levels above control of BASP2/GAP43 is associated with growth cone damage; the presence of levels above control of Nesprin-1 is associated with neuronal nuclear damage; the presence of levels above control of calmodulin regulated spectrin-associated protein 1, calmodulin regulated spectrin-associated protein 2, calmodulin regulated spectrin-associated protein 3, Tau-441, or Tau-758 is associated with axonal injury; the presence of levels above control of microtubule associated protein 6 is associated with dendritic damage; and the presence of levels above control of chondroitin sulfate proteoglycan 4, neurocan or brevican is associated with brain extracellular matrix damage; to determine the damaged central nervous system anatomical areas, cell types and subcellular structures in a subject associated with the one or more proteins, protein breakdown products and/or peptide fragments of present above control levels in the fluid biological sample.

Preferred embodiments of the invention are those wherein the trauma is cortical impact, closed head injury, blast overpressure induced brain injury, or concussion, and wherein the fluid biological sample is cerebrospinal fluid, blood, plasma, serum, wound fluid, or biopsy, necropsy or autopsy samples of brain tissue, spinal tissue, retinal tissue, and/or nerves.

Embodiments of the invention include a diagnostic kit comprising (a) detection agents for antibody, aptamer or mass spectrometry detection methods for detection of one or more peptide fragments selected from the group consisting of

Tau-441 peptides:
(SEQ ID NO: 471)
AEPRQEFEVMEDHAGTYGLG;
(SEQ ID NO: 472)
AAQPHTEIPEGTTAEEALEDEAAGHVTQARMVS;
(SEQ ID NO: 473)
LSKVTSKCGSLG;
(SEQ ID NO: 474)
SPQLATLADEVSASLAK;
(SEQ ID NO: 475)
TLADEVSASLAKQGL;
Tau-758 (Tau-G) peptides:
(SEQ ID NO: 476)
PQLKARMVSKSKDGTGSDDKKAKTSTRSSA;
(SEQ ID NO: 477)
SPKHPTPGSSDPLIQPSSPAVCPEPPSSPKYVSSVTSRTGSSGAKEM;
(SEQ ID NO: 478)
PPSSPKYVSSVTSRTGSSGAKEMKLKGADGKTKIATPRGAA;
(SEQ ID NO: 479)
SVTSRTGSSGAKEMKLKGADGK;
(SEQ ID NO: 480)
SPKHPTPGSSDPLIQPSSPAVCPE;
(SEQ ID NO: 481)
PPSSPKYVSSVTSRTGSSGAKEMKL;
Neurogranin peptides:
(SEQ ID NO: 482)
ILDIPLDDPGANAAAAKIQAS(p)8FRGHMARKKIKSGERGRKGPGPGGP
GGA (*(p)=phospho-Serine);
(SEQ ID NO: 483)
ILDIPLDDPGANAAAAKIQASFRGHMARKKIKSGERGRKGPGPGGPGGA;
(SEQ ID NO: 484)
DDDILDIPLDDPGANAAAAKIQAS(p)FR;
(SEQ ID NO: 485)
DDDILDIPLDDPGANAAAAKIQASFR;
(SEQ ID NO: 486)
PGANAAAAKIQAS(p)FRGHMARKKIKSGERGRKGPGPGG;
(SEQ ID NO: 487)
PGANAAAAKIQASFRGHMARKKIKSGERGRKGPGPGG;
Vimentin peptides:
(SEQ ID NO: 488)
NVKMALDIEIAT;
(SEQ ID NO: 489)
LLEGEESRISLPLPNFSSLNLR;
(SEQ ID NO: 490)
NVKMALDIEIATYRKLLEGEESRISLPLPNFSSLNLRETNLDSLPLVDTH
SKR;
(SEQ ID NO: 491)
TLLIKTVETRDGQVIN;
(SEQ ID NO: 492)
MSTRSVSSSS YRRMFGGPGT ASRPSSSRSY VTTSTRTYSL
GSALRPSTSR SLYASSPGGV YATRSSAVRL RSSVP;
(SEQ ID NO: 493)
STRSVSSSSYRRMFGGPGTASRPSSSRSYVTTSTRTYSLGSALR;
MBP peptides:
(SEQ ID NO: 494)
HGSKYLATASTMD;
(SEQ ID NO: 495)
HGSKYLATASTMDHARHGFLPRHRDTGILDSIGR;
(SEQ ID NO: 496)
GRTQDENPVVHFFKNIVTPRTPPPSQGKGRGLSLSRF;
(SEQ ID NO: 497)
HKGFKGVDAQGTLS;
Golli-MBP1 isoform peptides:
(SEQ ID NO: 498)
HAGKRELNAEKASTNSETNRGESEKKRNLGELSRTT;
(SEQ ID NO: 499)
NAWQDAHPADPGSRPHLIRLFSRDAPGREDNTFKDRPSESDE;
GFAP peptides:
(SEQ ID NO: 500)
ITSAARRSYVSSGEMMVGGLAPGRRLGPGTRLSLARMP;
(SEQ ID NO: 501)
YVSSGEMMVGGLAPGRRLGPGTRLS;
(SEQ ID NO: 502)
RSYVSSGEMMVGGLAPGRRLGP;
(SEQ ID NO: 503)
AARRSYVSSGEMMVGGLAPGRRLGPGTRLSLARMPPPLPTR;
(SEQ ID NO: 504)
GEENRITIPVQTFSNLQIRETSLDTKSV;
(SEQ ID NO: 505)
QTFSNLQIRETSLDTKSVSEGHLKRNIVVKTVEMR;
(SEQ ID NO: 506)
DGEVIKES;
(SEQ ID NO: 507)
DGEVIKE;
(SEQ ID NO: 508)
DGEVIKESKQEHKDVM;
(SEQ ID NO: 509)
TKYSEATEHPGAPPQPPPPQQ;
(SEQ ID NO: 510)
QLPTVSPLPRVMIPTAPHTEYIESS.
Complement C1q subcomponent subunit B (D6R934)
peptide:
(SEQ ID NO: 701)
HGEFGEKGDPGIPG;
Complement C3 (P01024) peptide:
(SEQ ID NO: 702)
HWESASLL;
(SEQ ID NO: 703)
VKVFSLAVNLIAI;
Complement C5 (P01031) peptide:
(SEQ ID NO: 705)
VTcTNAELVKGRQ;
Complement C1s (P09871) peptide:
(SEQ ID NO: 706)
IISGDTEEGRLcGQRSSNNPHSPIVE;
Complement receptor type 1 CR1 (E9PDY4 ) peptides:
(SEQ ID NO: 704a)
KTPEQFPFAS;
(SEQ ID NO: 704b)
SCDDFMGQLLNGRVLFPVNLQLGAK;
Microtubule-associated Protein 6 (MAP6) (Q7TSJ2)
peptides:
(SEQ ID NO: 179)
TKYSEATEHPGAPPQPPPPQQ;
(SEQ ID NO: 180)
QLPTVSPLPRVMIPTAPHTEYIESS;
Synapsin I (SYNI)(P17600-1 or P17600-2) peptides:
(SEQ ID NO: 181)
QDEVKAETIRS;
Synapsin II (SYN2)(Q9277-1 or Q64332 ) peptides:
(SEQ ID NO: 182)
SQSLTNAFSFSESSFFRS;
Synapsin III (SYN3)(Q14994-1 or P07437 )
peptides:
(SEQ ID NO: 183)
DWSKYFHGKKVNGEIEIRV;
and
((SEQ ID NO: 184)
GEHVEEDRQLMADLVVS

(b) an analyte protein, protein breakdown product, or peptide fragment to serve as internal standard and/or positive control; and (c) a signal generation coupling component.

### BRIEF DESCRIPTION OF THE FIGURES

The following figures are included to further demonstrate certain non-limiting embodiments of the present invention. The invention may be better understood by reference to one or more of these drawings in combination with the detailed description of specific embodiments presented herein.

FIG. 1 is a schematic diagram showing the production of higher molecular weight protein breakdown products (PBP) and lower molecular weight peptide fragments (PF) after traumatic injury to the central nervous system or extracellular matrix, including higher molecular weight protein breakdown products (also referred to as PBP) over about 10,100-100,000 Da and low molecular weight peptide fragments (PF) of about 1,000-10,000 Da.

FIG. 2 is a schematic diagram showing the steps for identifying the PBP and PF of this invention.

FIG. 3 is a series of photographs showing representative brain areas that produce Cortexin-1, Striatin, and MBP/Golli-MBP upon traumatic injury, based on their respective mRNA expression.

FIG. 4 is a diagram showing cetain subcellular compartments and the protein breakdown products which are produced in them upon injury. BBB indicates blood-brain barrier.

FIG. 5 is a graph showing LC/MS characterization (spectrum) of neurogranin (NGRN) proteolytic breakdown products (PF and concurrent PBP formation) in mouse brain lysate after TBI in mice. The figure shows an MS/MS spectrum of the NRGN PF PGANAAAAKIQASFRGHMARKKIKSGERGRKGPGG; NRGN aa 24-63; SEQ ID NO:1) released from ipsilateral cortex CCI (day 1) after injury in mice. The tandem mass spectrum shows the fragment (product) ions with observed b⁺⁻ and y⁺⁻type ions shown in italics and underline, respectively. The NRGN peptide (precursor) ion, shown in bold, was observed as a charge of +3 for monoisotopic mass-to-charge ratio (m/z) 1245.87.

FIG. 6 is a graph showing an MS/MS spectrum of the NRGN PF DDDILDIPLDDPGANAAAAKIQASFR; NGRN aa 16-38; SEQ ID NO:2) released from ipsilateral cortex CCI (day 7) after injury in mice. The figure displays the fragment ions for this peptide, charge +3, monoisotopic m/z 904.30 Da.

FIG. 7A and FIG. 7B are photographs of a western blot (FIG. 7A) showing the ipsilateral cortex profile of the NRGN fragmentation pattern at different time points (day 1 and day 7, as indicated) after CCI and repetitive closed head injury (rCHI) in mice and a graph (FIG. 7B) showing a densitometric quantitation of the intact and PBP of NRGN. Error bars represent the standard error of the mean (n=3). * shows statistical significance over naive mice (p value <0.05), 2-tailed unpaired T-test.

FIG. 7C and FIG. 7D are photographs of a western blot (FIG. 7C) showing the ipsilateral hippocampal profile of the NRGN fragmentation pattern at different time points (day 1 and day 7, as indicated) after CCI and rCHI in mice and a graph (FIG. 8D) showing a densitometric quantitation of the intact and PBP of NRGN. Error bars represent the standard error of the mean (n=3). * shows statistical significance over naive mice (p value <0.05), 2-tailed unpaired T-test.

FIG. 8A shows a characterization of Vimentin (VIM) PFs and concurrent PBP formation in mouse cortical lysate after TBI in mice. The figure shows an MS/MS spectrum of the VIM PF GSGTSSRPSSNRSYVTTSTRTYSLGSALRPSTSR; VIM aa 17-50; SEQ ID NO:10), charge +2, monoisotopic m/z 1902.83 Da, displaying the fragment ions for this peptide.

FIG. 8B is an MS/MS spectrum of a VIM PF released from ipsilateral cortex CCI (day 1) injury in mice. The figure shows an MS/MS spectrum for the VIM PF NLESLPLVDTHSKRTLLIKTVETRDGQVINE (VIM aa 426-456; SEQ ID NO:11), charge+3, monoisotopic m/z 1227.03 Da, displaying the fragment ions for this peptide.

FIG. 9A and FIG. 9B show the profile of the VIM fragmentation pattern at different time points (day 1, day 3 and day 7) as indicated, after CCI in mouse cortex. FIG. 9D is a western blot showing the PBPs of VIM using an internal epitope antibody (Abcam ab92547) with internal loading control β-actin (43 kDa). Intact VIM appears as a 50 kDa band, while major PBPs appear as 48 and 38 kDa bands. FIG. 9E is a densitometric quantitation of the intact VIM protein and its PBPs. Error bars represent the standard error of the mean (N=3). * indicates statistical significance over naive (p-value <0.05) (2 tailed unpaired T-test).

FIG. 9C and FIG. 9D show the profile of the VIM fragmentation pattern at different time points (day 1, day 3 and day 7) as indicated, after CCI in mouse hippocampus. FIG. 9F is a western blot showing the PBPs of VIM using an internal epitope antibody (Abcam ab92547) with internal loading control β-actin (43 kDa). Intact VIM appears as a 50 kDa band, while major PBPs appear as 48 and 38 kDa bands. FIG. 9G is a densitometric quantitation of the intact VIM protein and its PBPs. Error bars represent the standard error of the mean (N=3). * indicates statistical significance over naive (p-value <0.05) (2 tailed unpaired T-test).

FIG. 10A presents an MS/MS spectrum of the mouse myelin basic protein PF KNIVTPRTPPP (aa 115-152; SEQ ID NO:48).

FIG. 10B is a western blot showing the myelin basic protein 10 kDa products, visualized with an epitope-specific antibody recognizing the peptide KNIVTPRTPPP (SEQ ID NO:195) and using internal loading of the control β-actin. FIG. 10C shows the densitometric quantitation of the 10 kDa myelin basic protein PF.

FIG. 10D is a western blot showing the myelin basic protein 10 kDa products, visualized with an epitope-specific antibody recognizing the peptide KNIVTPRTPPP (SEQ ID NO:195) and using internal loading of the control β-actin. FIG. 10E shows the densitometric quantitation of the 10 kDa myelin basic protein PF.

FIG. 11 presents an MS/MS spectrum for the brain acidic soluble protein 1 (BASP-1) PF EAPAAAASSEQSV (SEQ ID NO:78) released from a hippocampus lysate digestion with calpain-1. The figure shows the fragment ions for this peptide.

FIG. 12A the MS/MS spectra of several low molecular weight PFs produced from calpain digestion of human GFAP (a cellular protease that is hyperactivated after traumatic brain injury). The peptide sequences are provided. FIG. 12B is a schematic diagram showing the general structure of the GFAP protein. FIG. 12C shows the sequences of GFAP peptides from the N-terminus and C-terminus of GFAP.

FIG. 13A is a schematic drawing showing th PFs identified from a Tau-441 calpain

SEQ ID NO: 83
[M].AEPRQEFEVMEDHAGTY.[G],;
SEQ ID NO: 84
[M].AEPRQEFEVMEDHAGTYG.[L],;
SEQ ID NO: 85
[E].PRQEFEVMEDHAGTYG.[L],;
SEQ ID NO: 86
[G].DRKDQGGYTMHQDQEGSEEPGSETSDAK.[S],;
SEQ ID NO: 87
[K].ESPLQTPTEDGSEEPGSETSDAK.[S],;
SEQ ID NO: 88
[A].AAQPHTEIPEGTTAEEAGIGDTPSLEDEAAGHVT.[Q],;
SEQ ID NO: 89
[A].AQPHTEIPEGTTAEEAGIGDTPSLEDEAAGHVT.[Q],;
SEQ ID NO: 90
[T].EIPEGTTAEEAGIGDTPSLEDEAAGHVT.[Q],;
SEQ ID NO: 91
[T].EIPEGTTAEEAGIGDTPSLEDEAAGHVTq.[A],;
SEQ ID NO: 92
[G].TTAEEAGIGDTPSLEDEAAGHVT.[Q],;
SEQ ID NO: 93
[Q].TAPVPMPDLK.[N],;
SEQ ID NO: 94
[T].APAVPMPDLK.[N],;
SEQ ID NO: 95
[D].LSVTSKCGSLG.[N],;
SEQ ID NO: 96
[K].SEKLDFKDRVQ.[S],;
SEQ ID NO: 97
[F].RENAKAKTDHGAEIVYKSPVVSGDT.[S],;
SEQ ID NO: 98
[N].AKAKTDHGAEIVYKSPVVSGDT.[S],;
SEQ ID NO: 99
[A].KAKTDHGAEIVYKSPVVSGDT.[S],;
SEQ ID NO: 100
[K].TDHGAIVYKSPVVSGDT.[S],;
SEQ ID NO: 101
[G].AEIVYKSPVVSGDT.[S],;
SEQ ID NO: 102
[T].SPRHLSNVSSTGSIDMVDSPQLATLADEVS.[A],;
SEQ ID NO: 103
[T].SPRHLSNVSSTGSIDMVDSPQLA.[T],;
SEQ ID NO: 104
[S].STGSIDMVDSPQLA.[T],;
and
[S].ASLAKQGL.[-].

digestion. The sequences in the order shown are

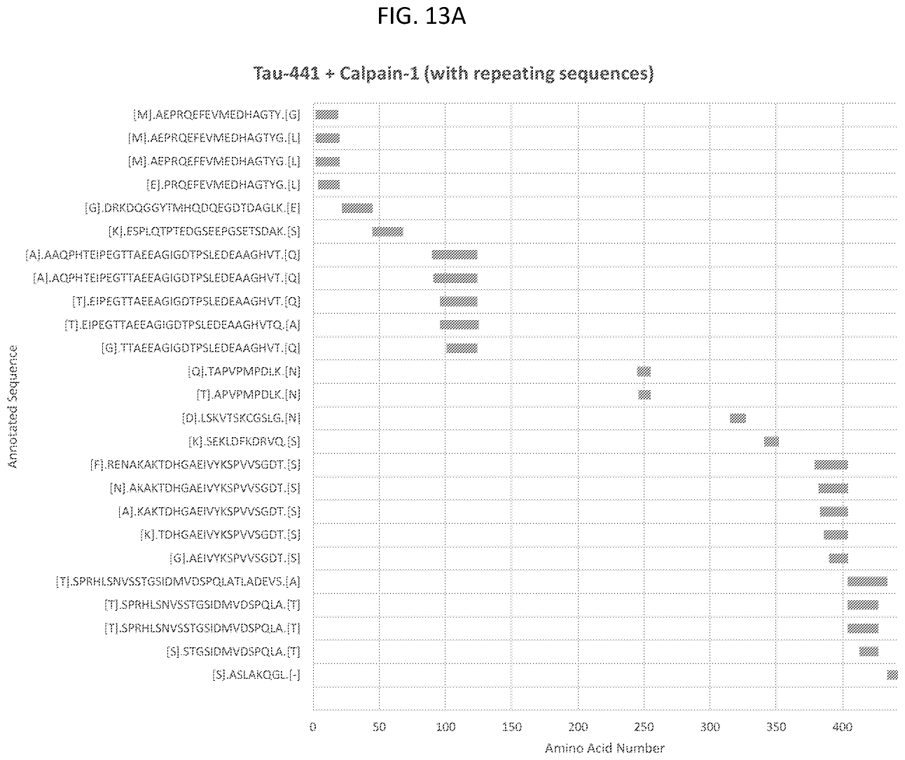

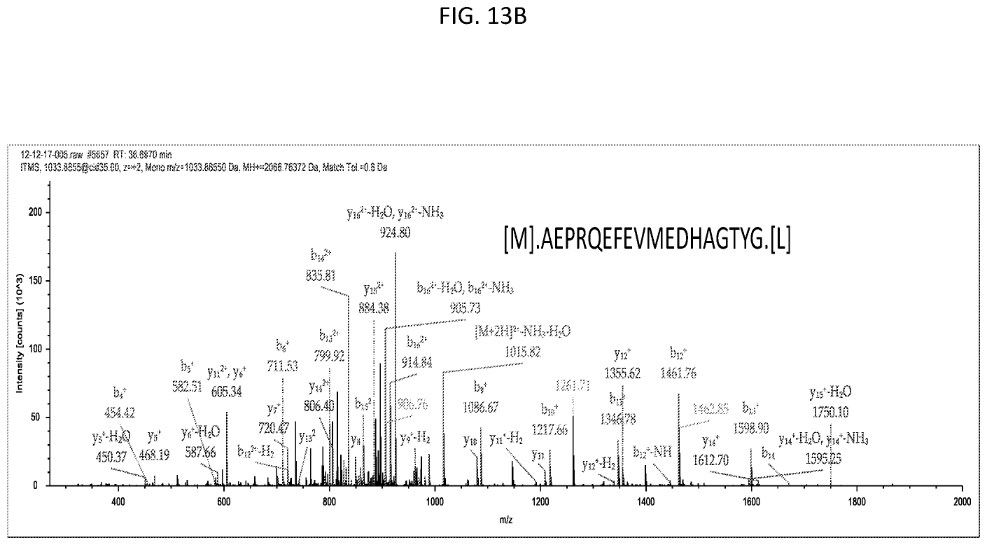

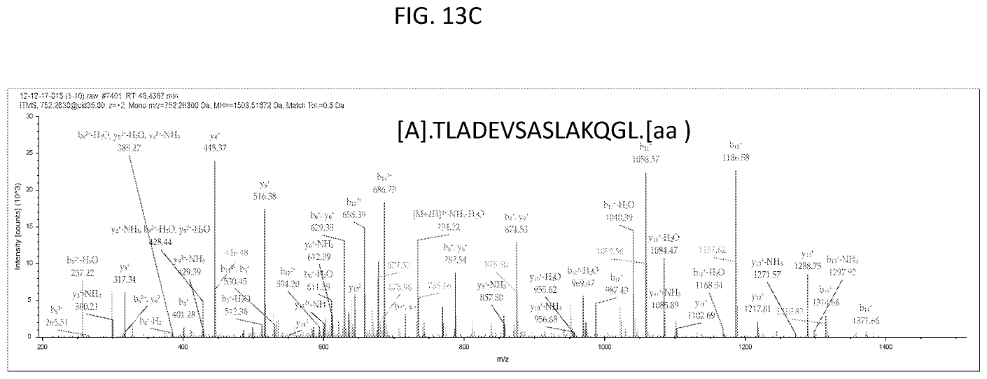

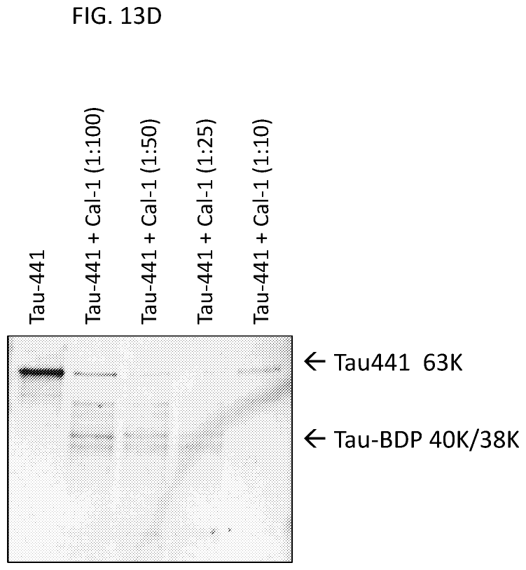

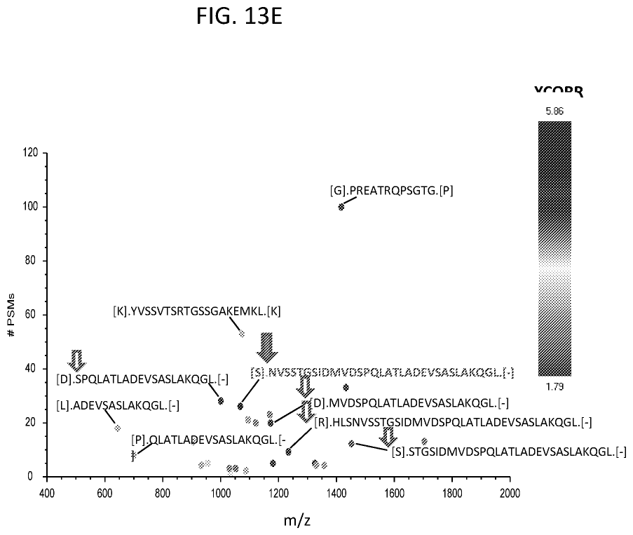

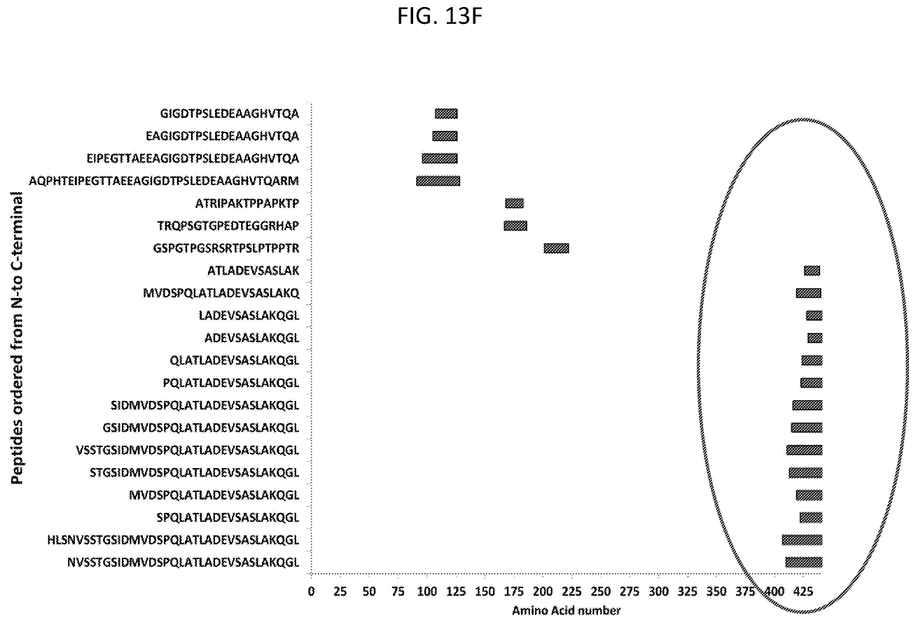

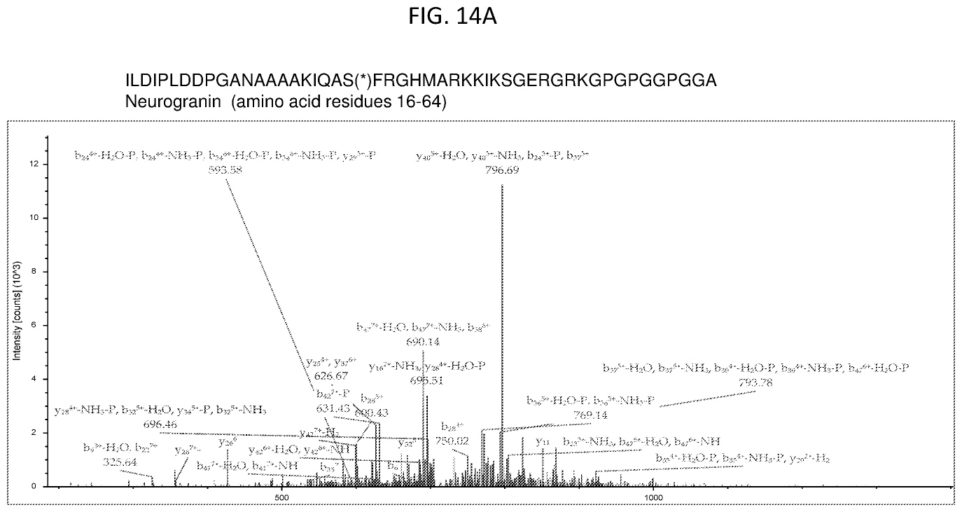

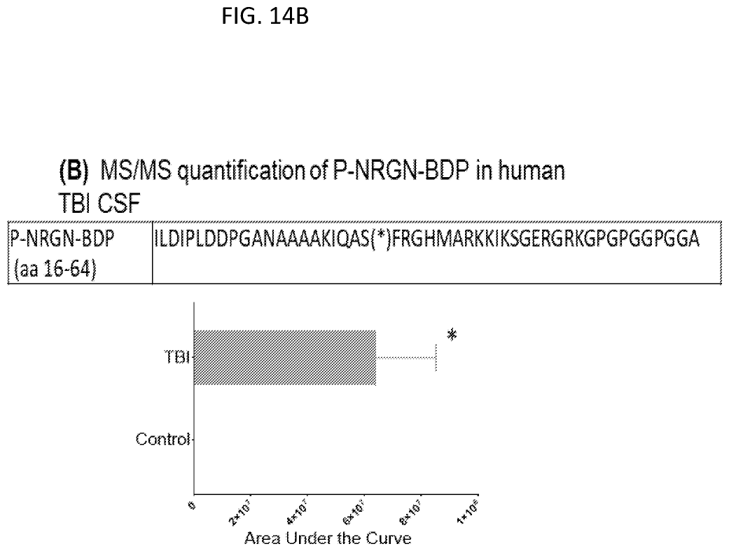

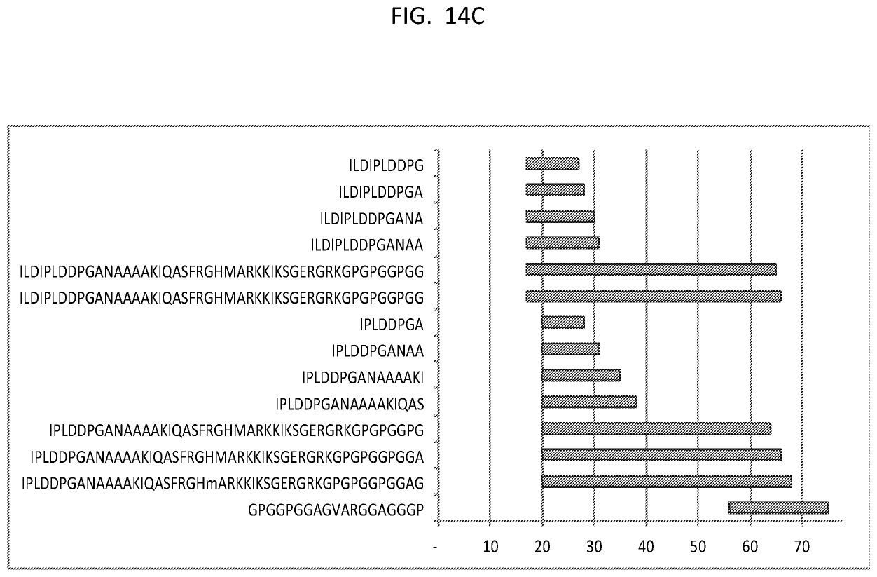

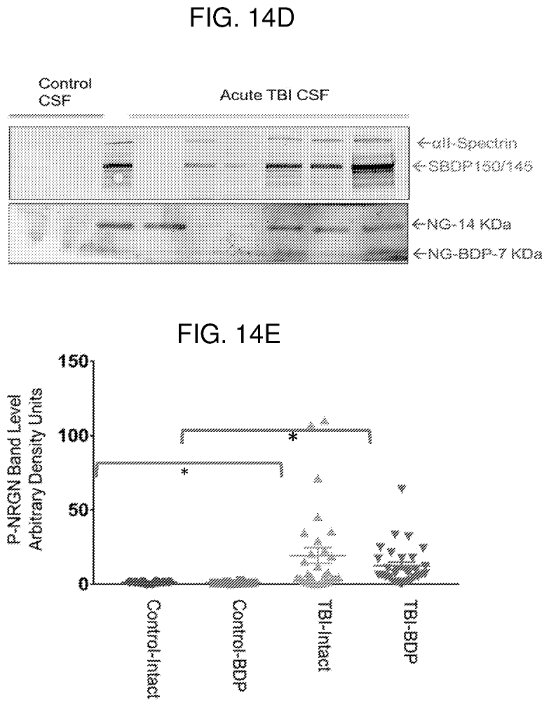

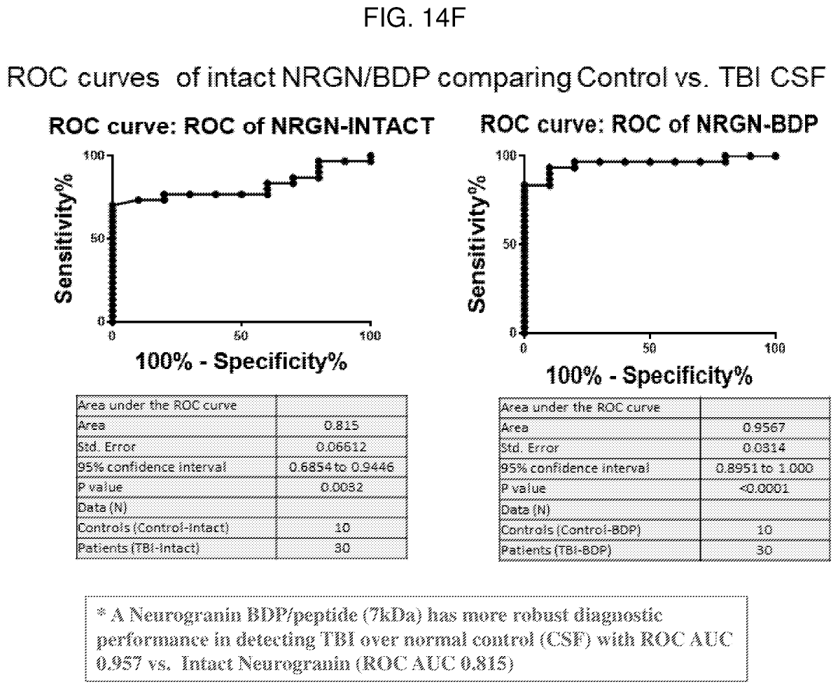

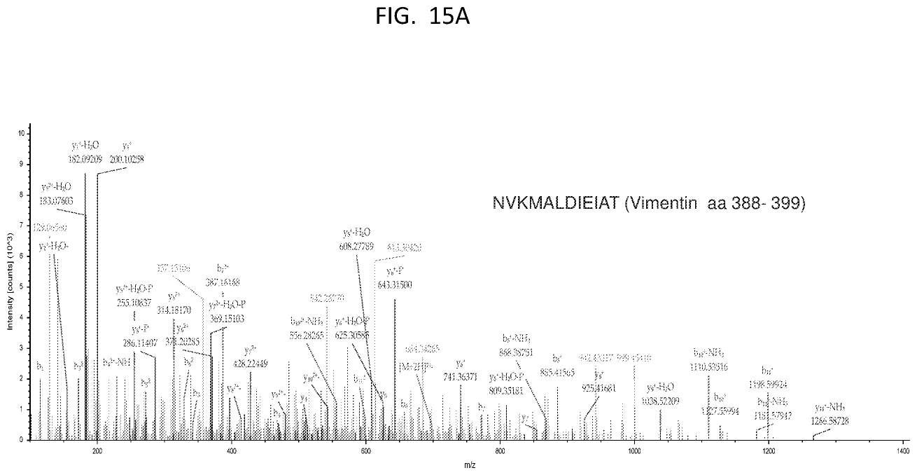

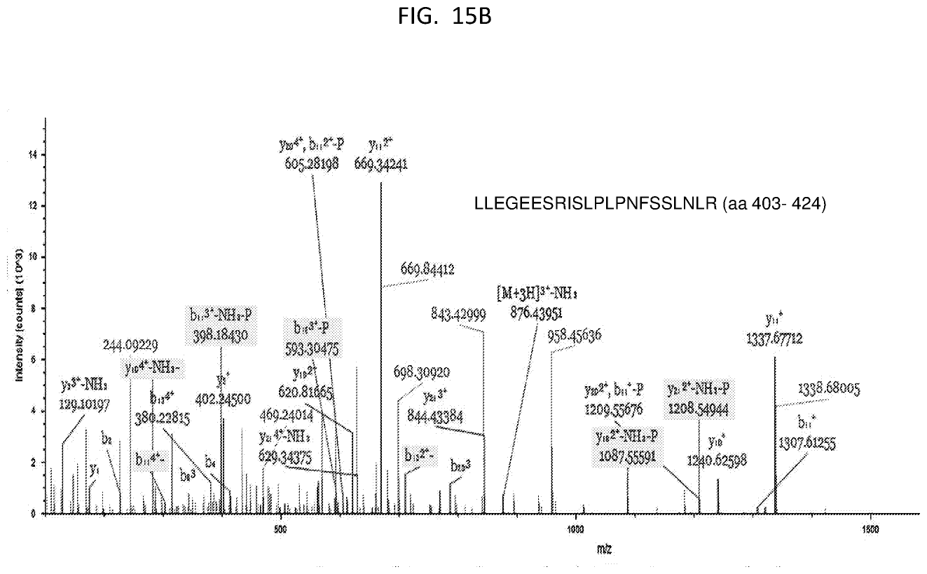

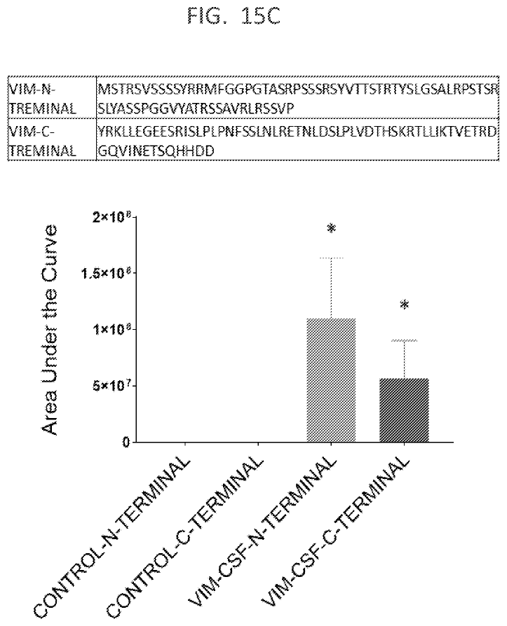

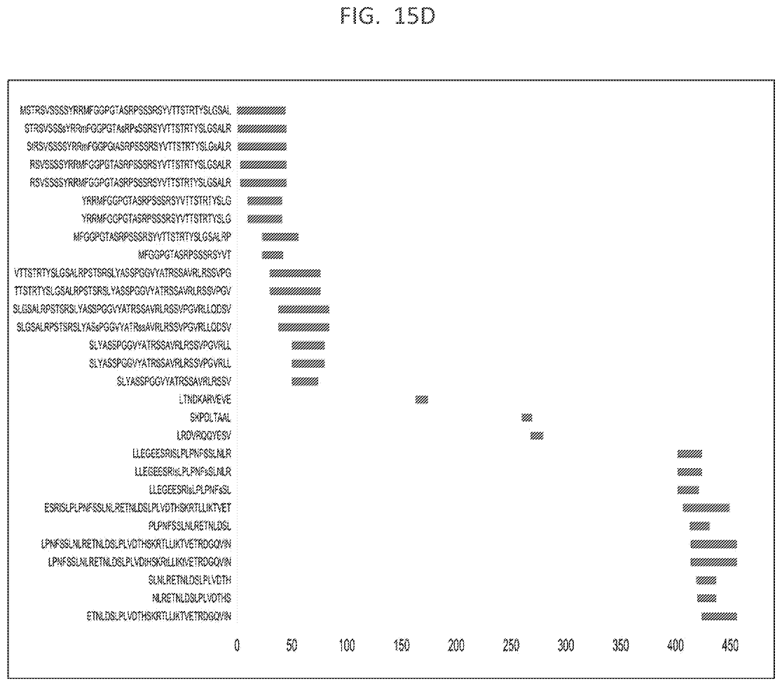

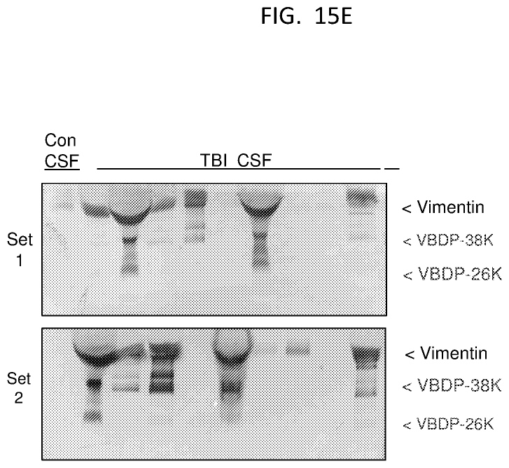

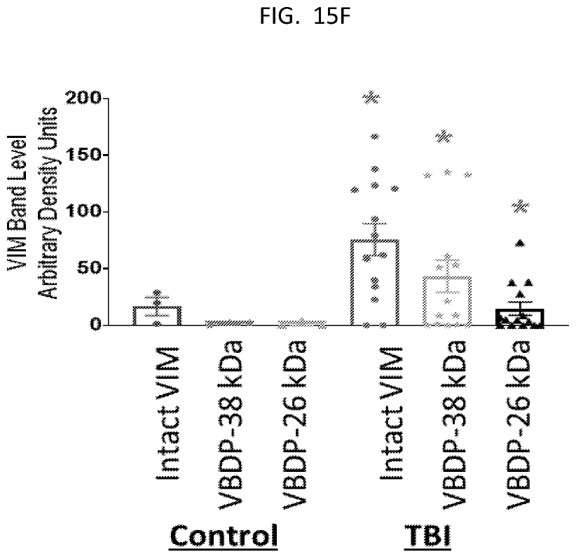

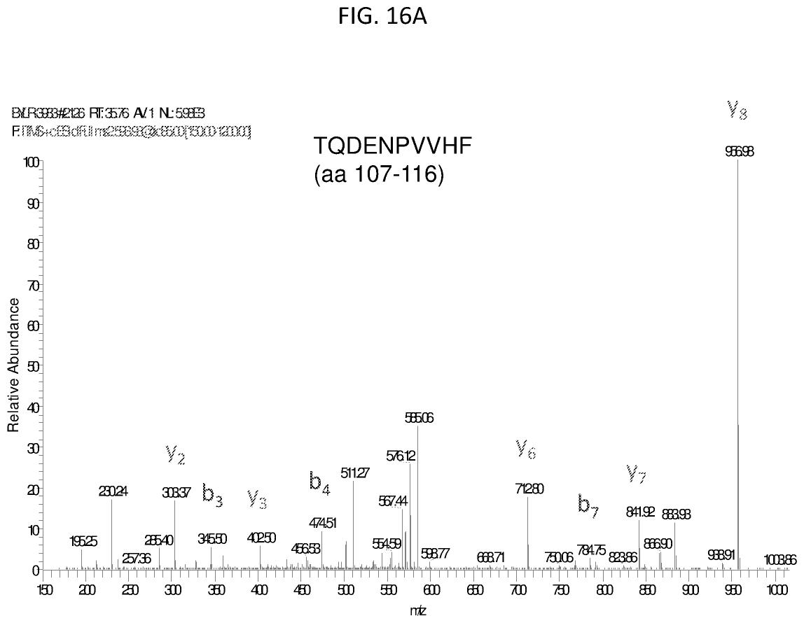

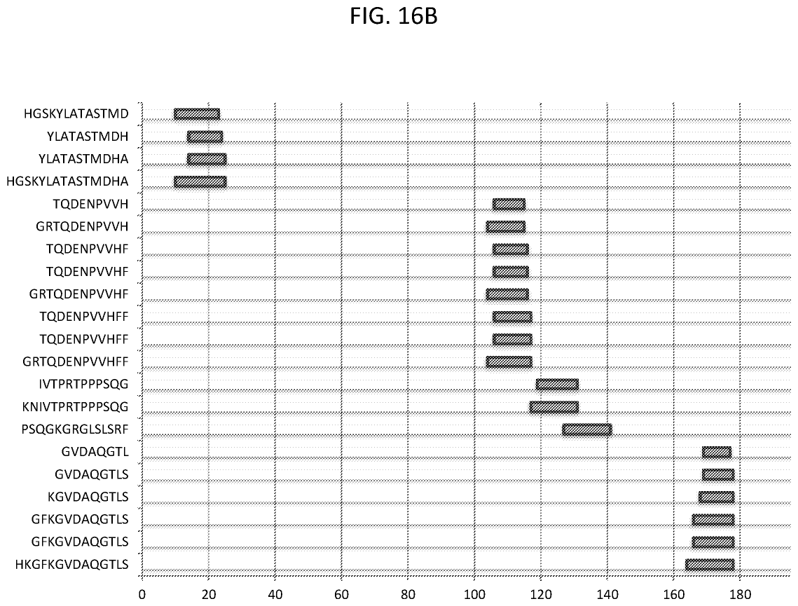

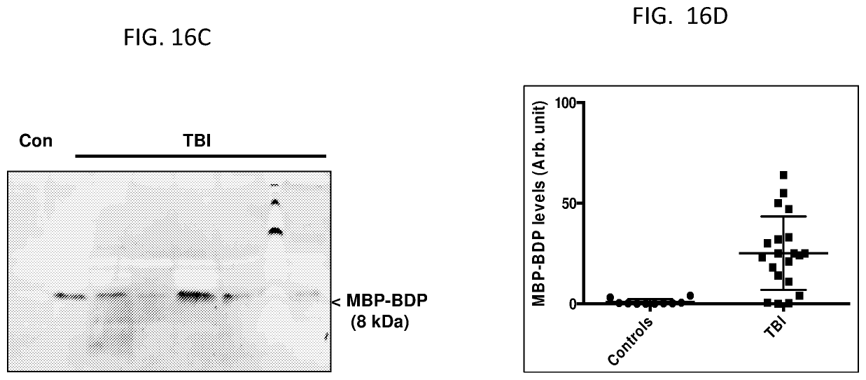

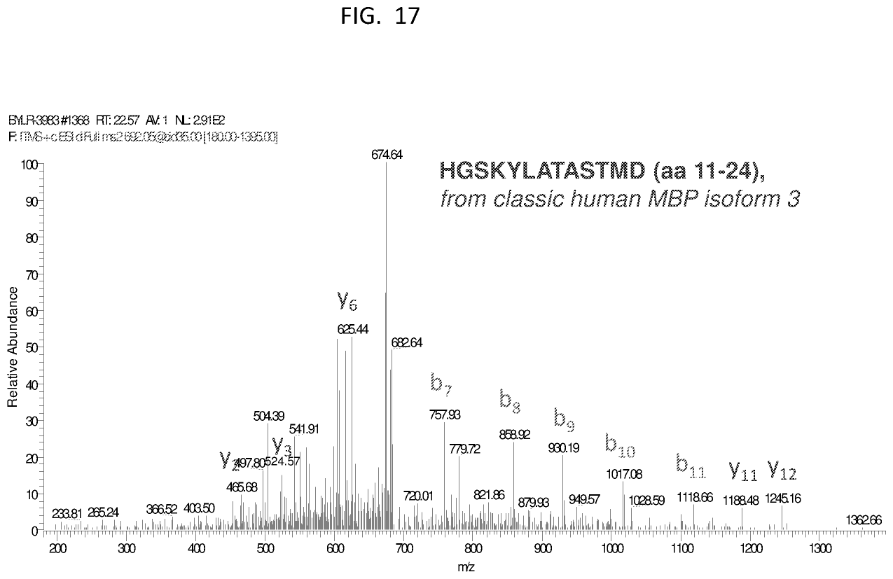

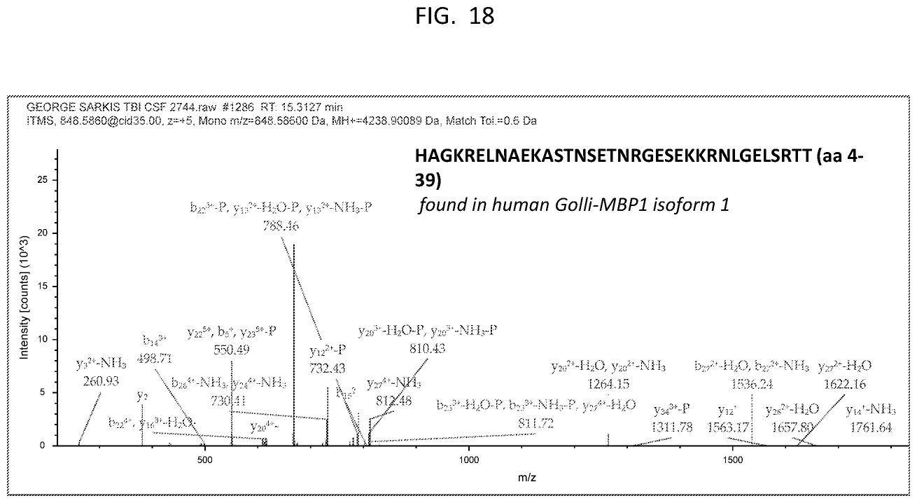

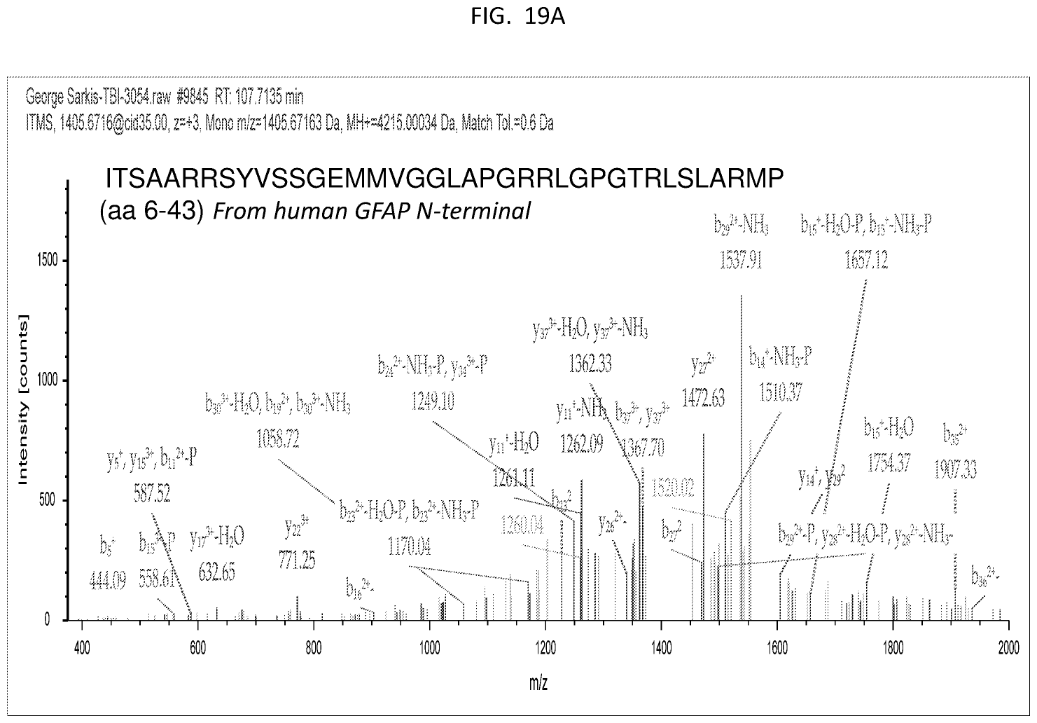

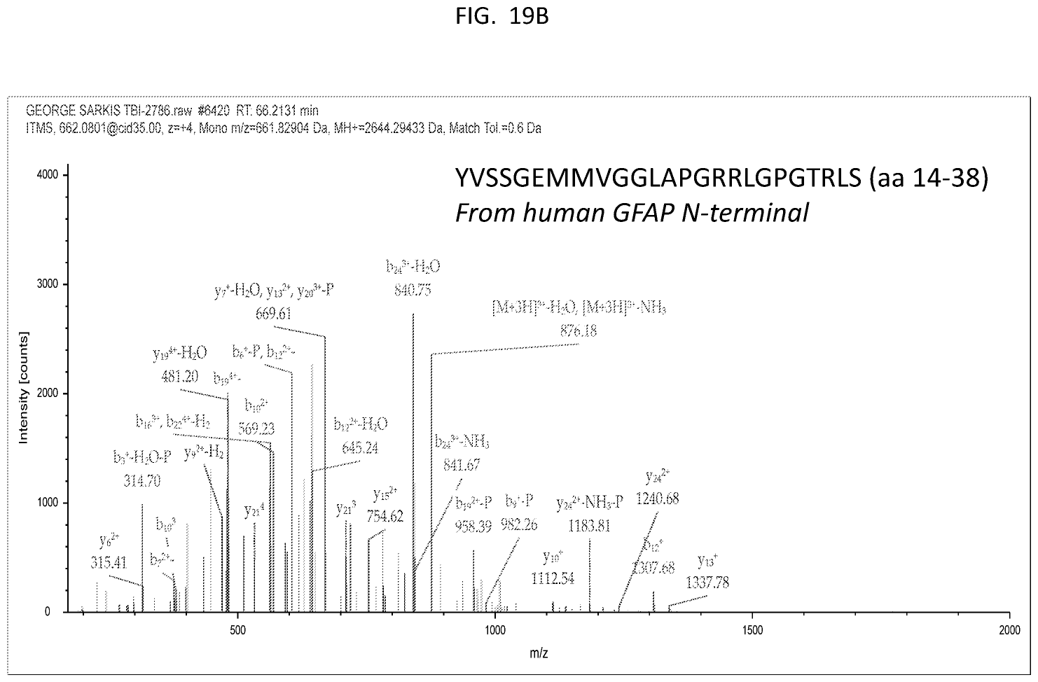

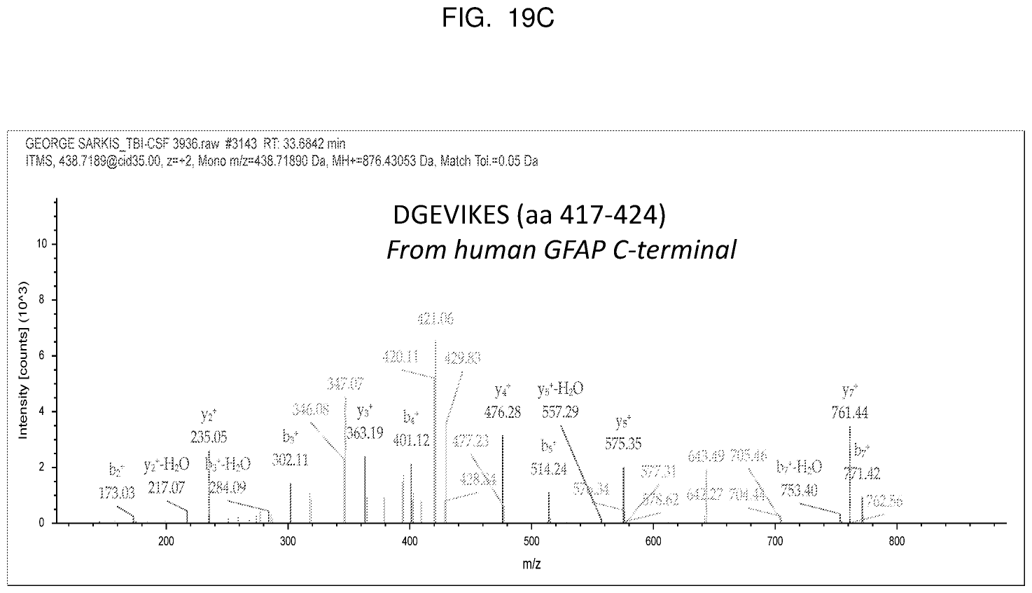

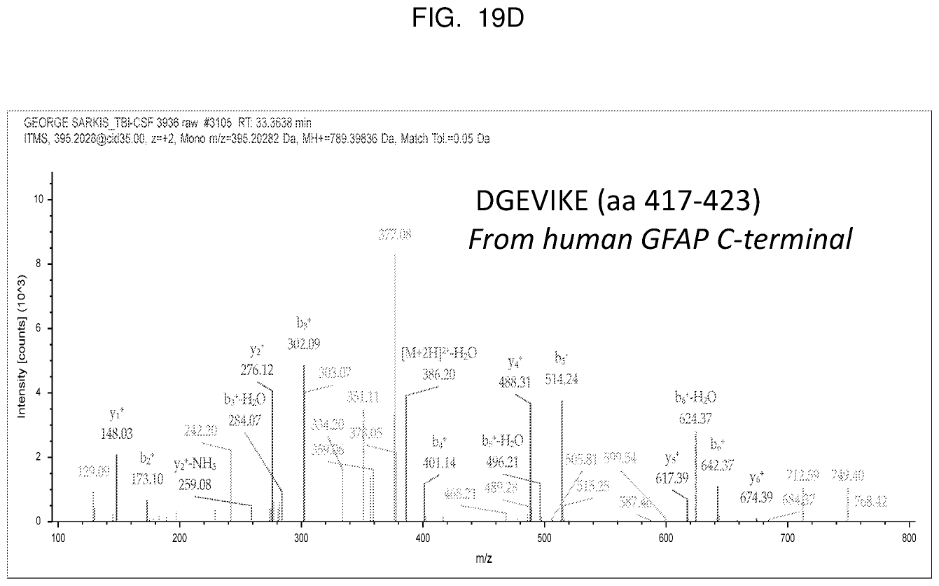

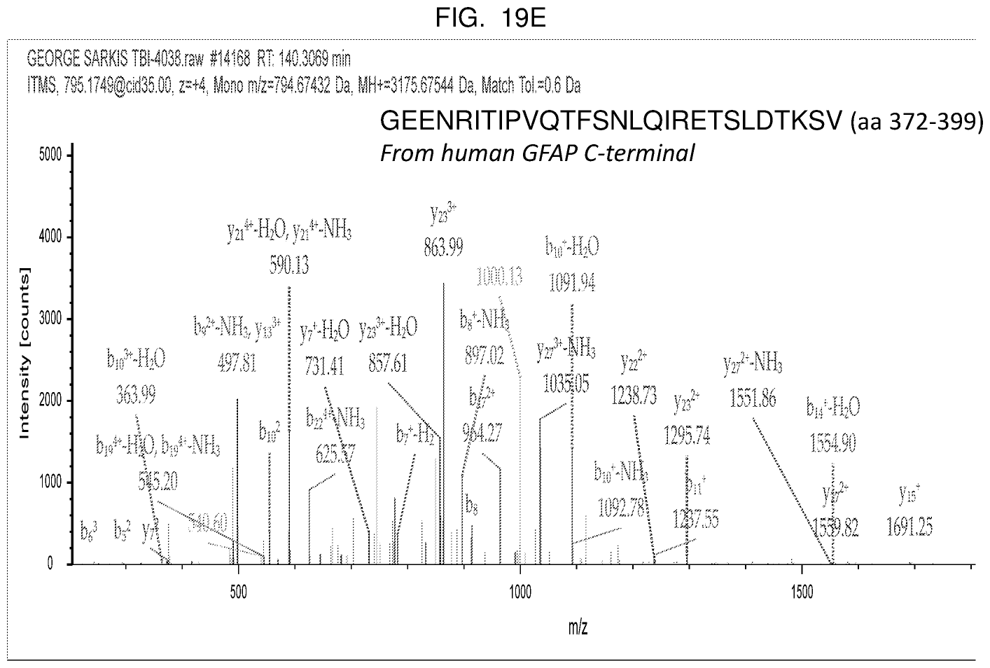

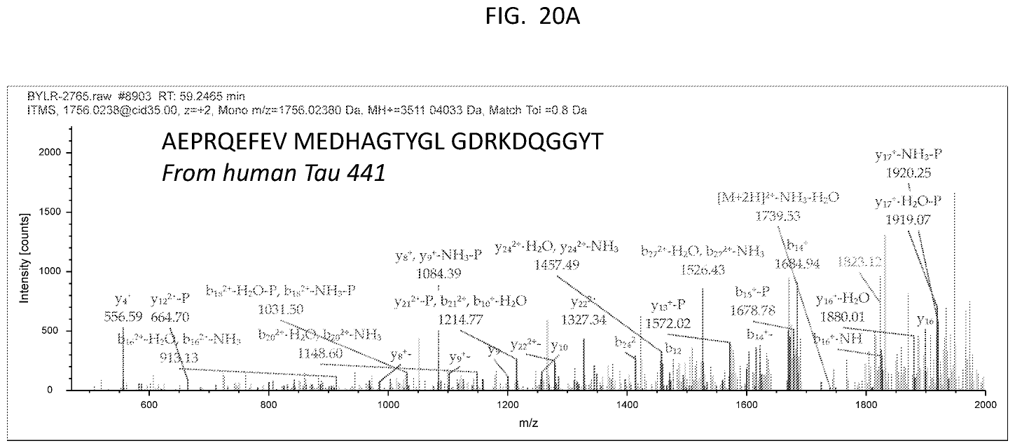

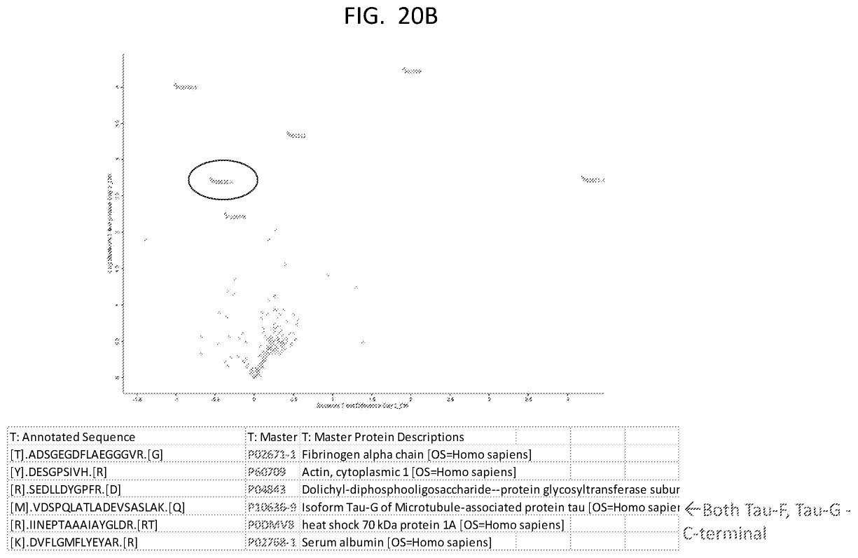

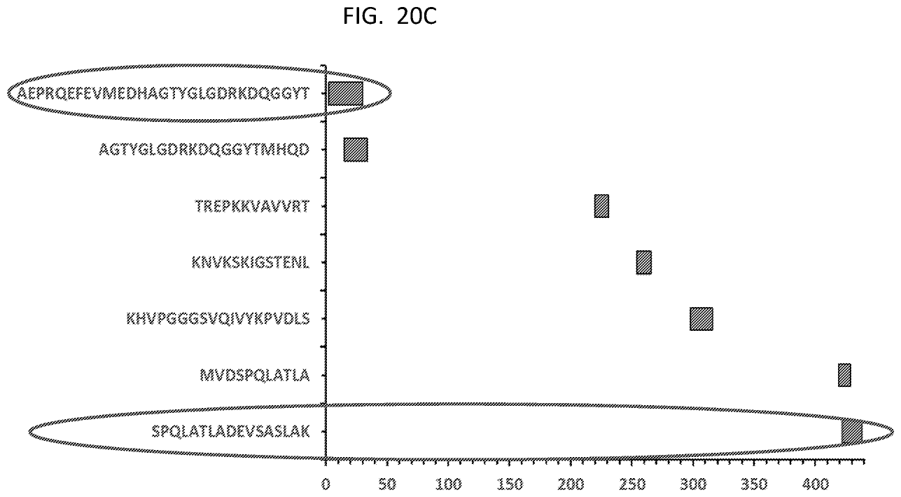

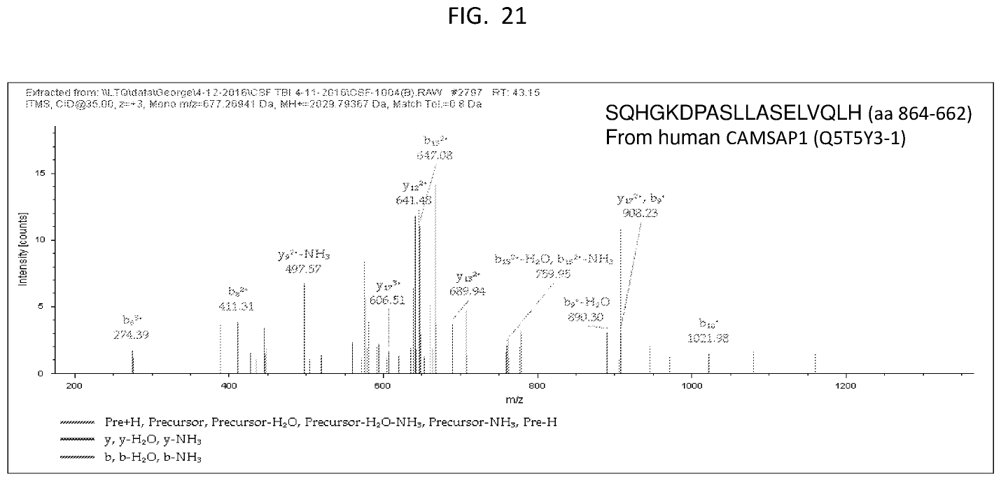

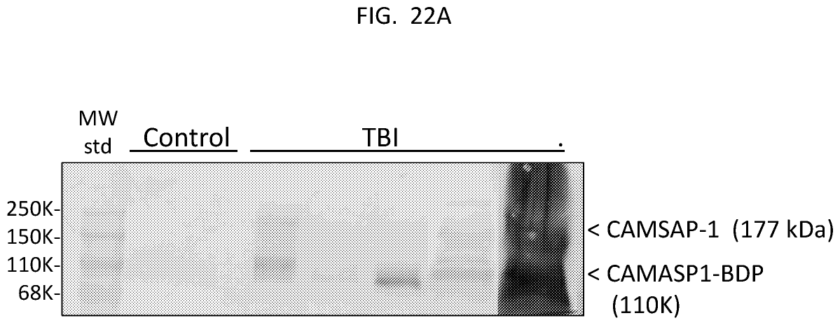

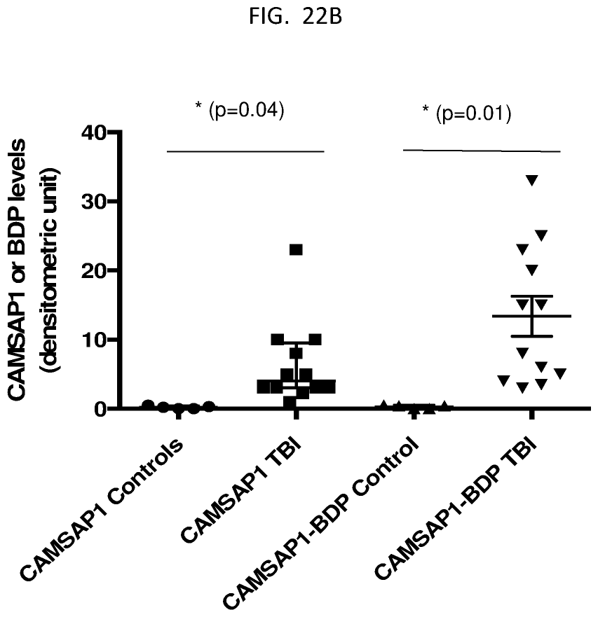

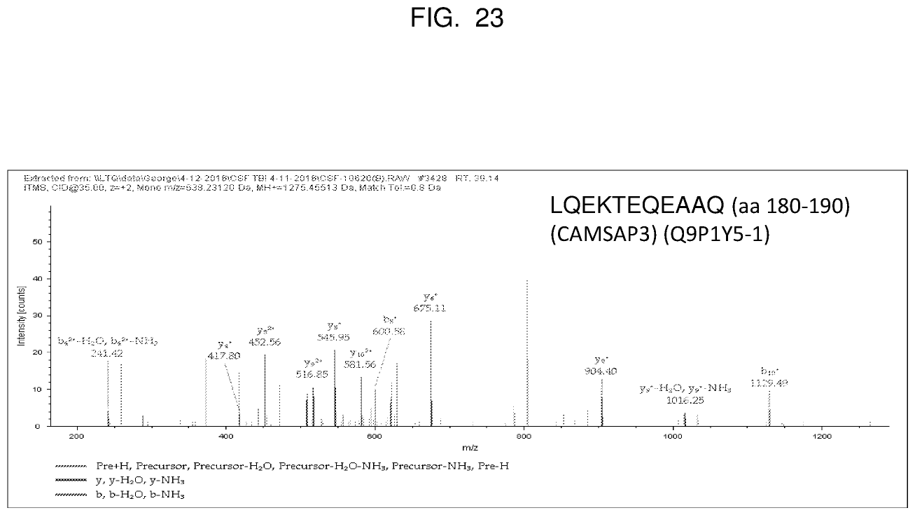

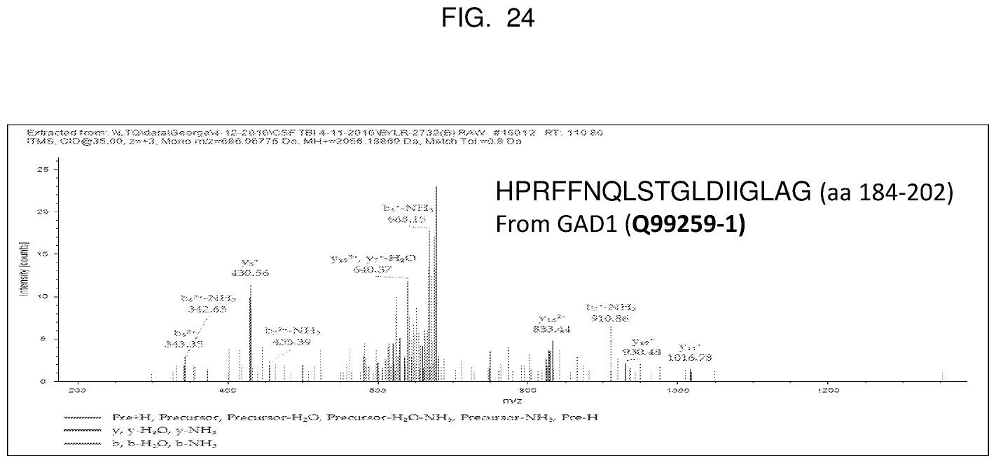

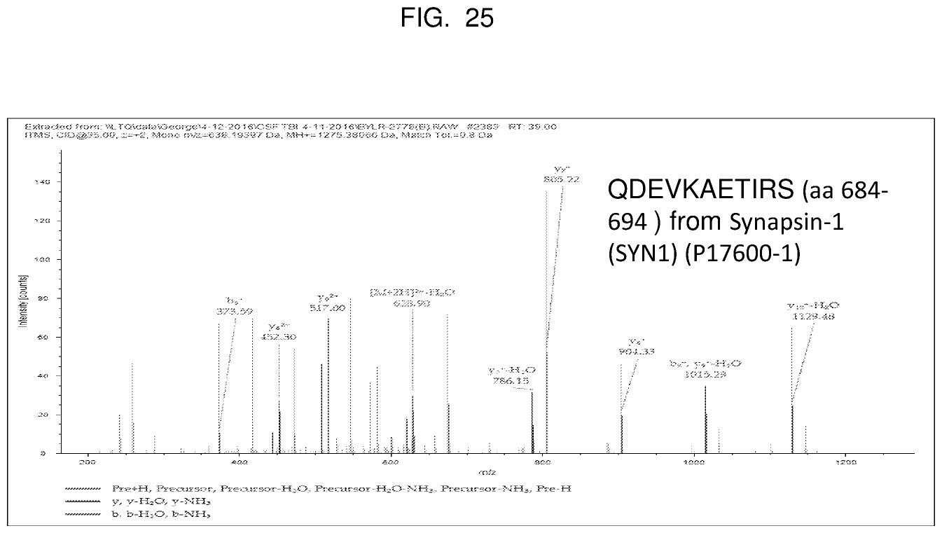

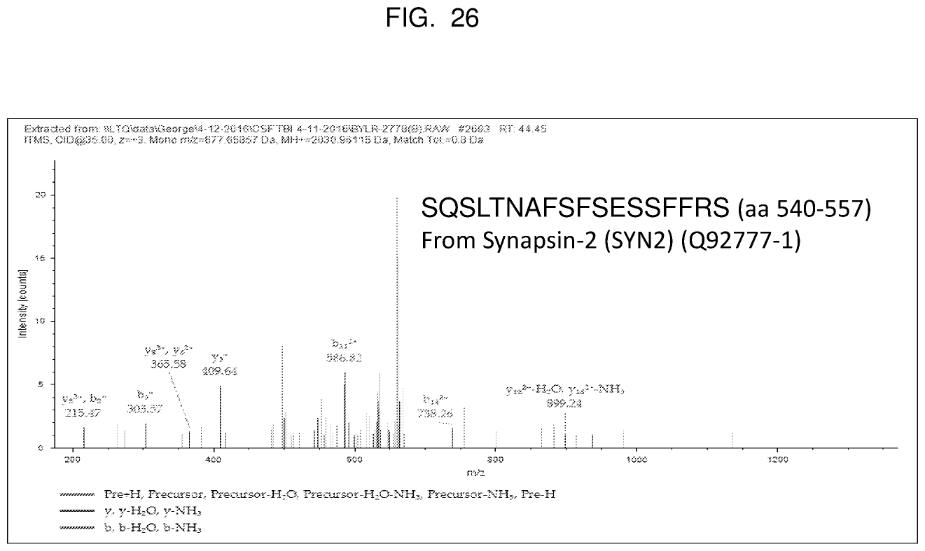

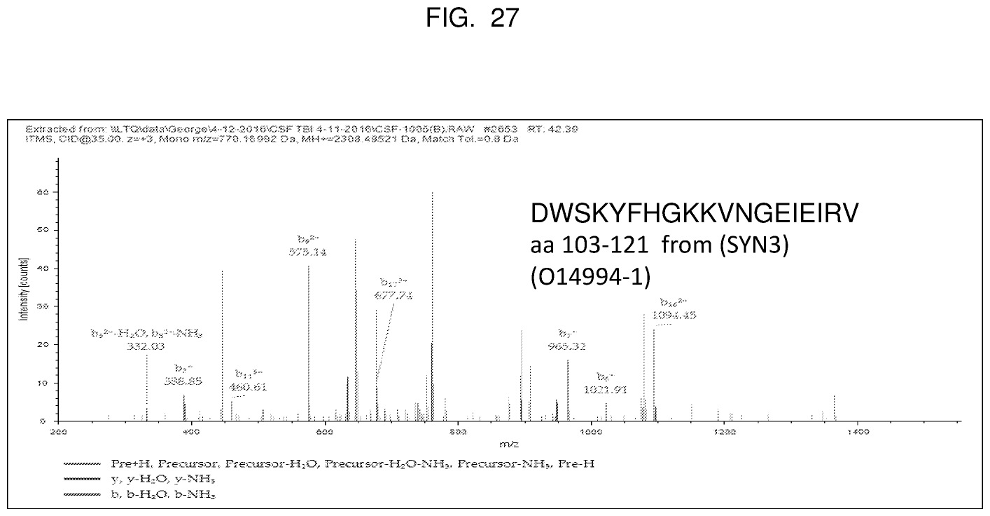

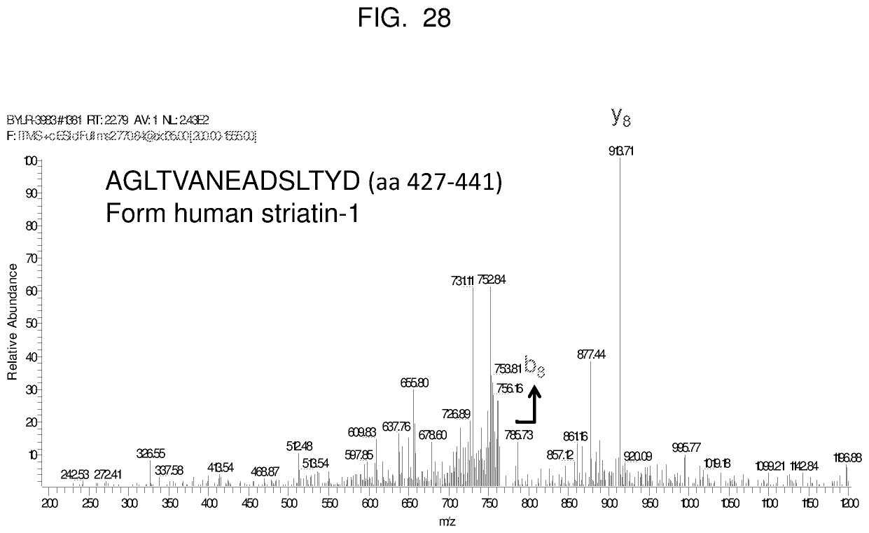

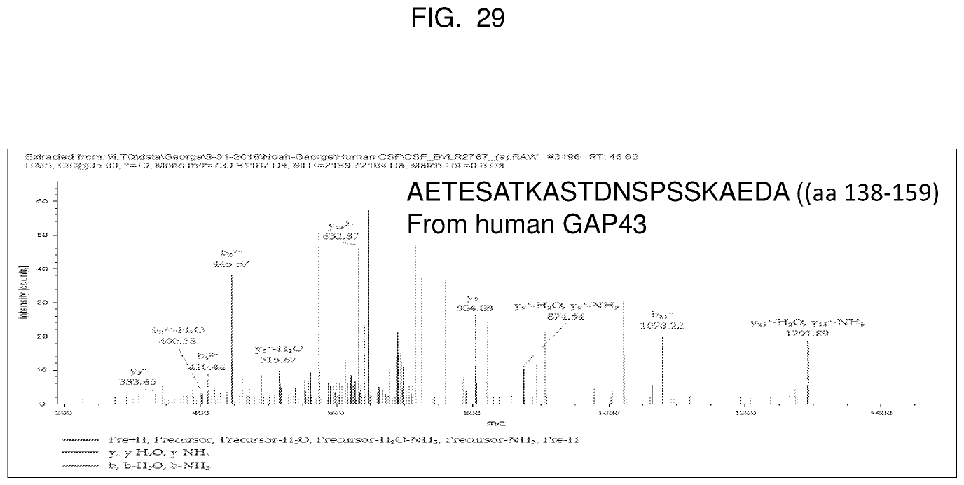

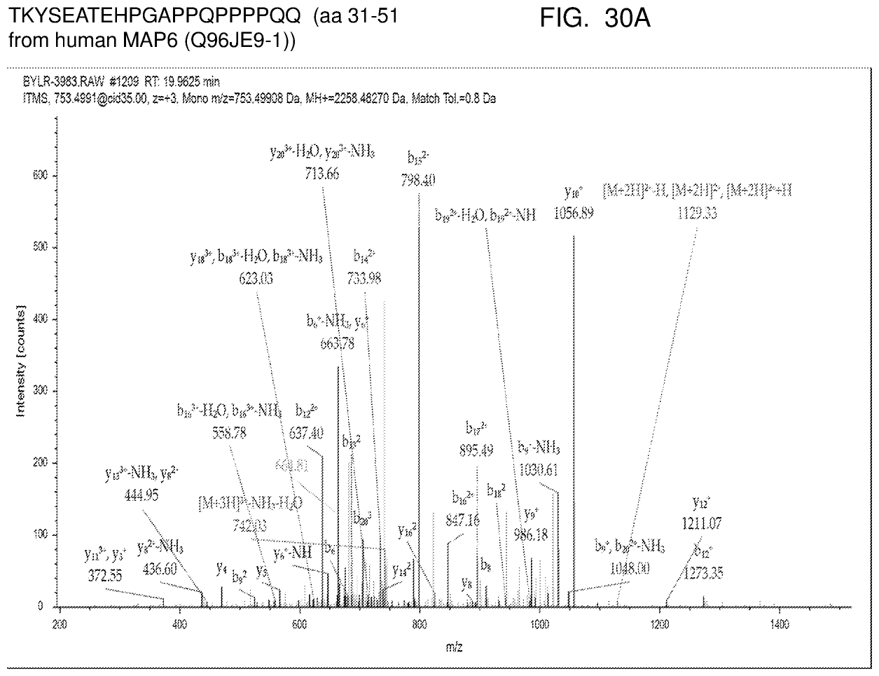

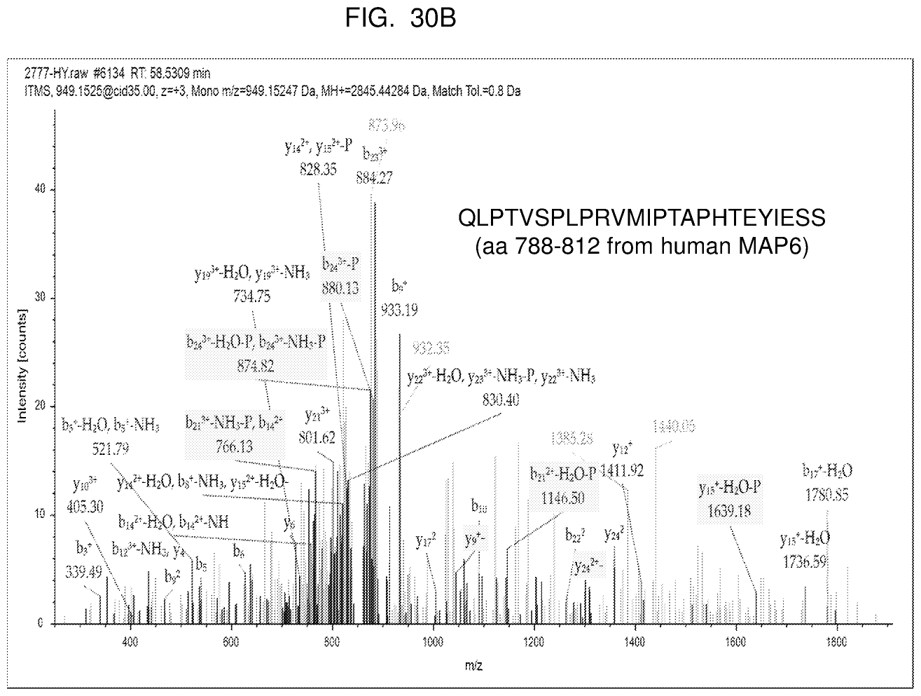

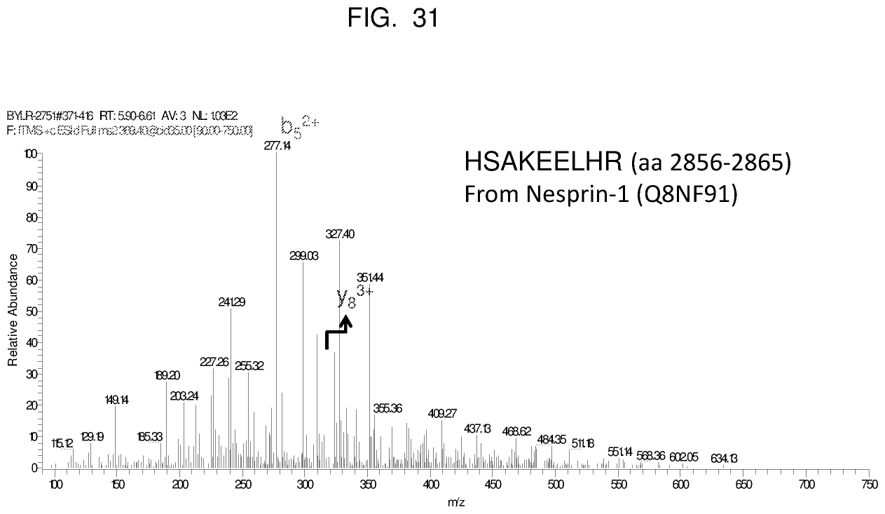

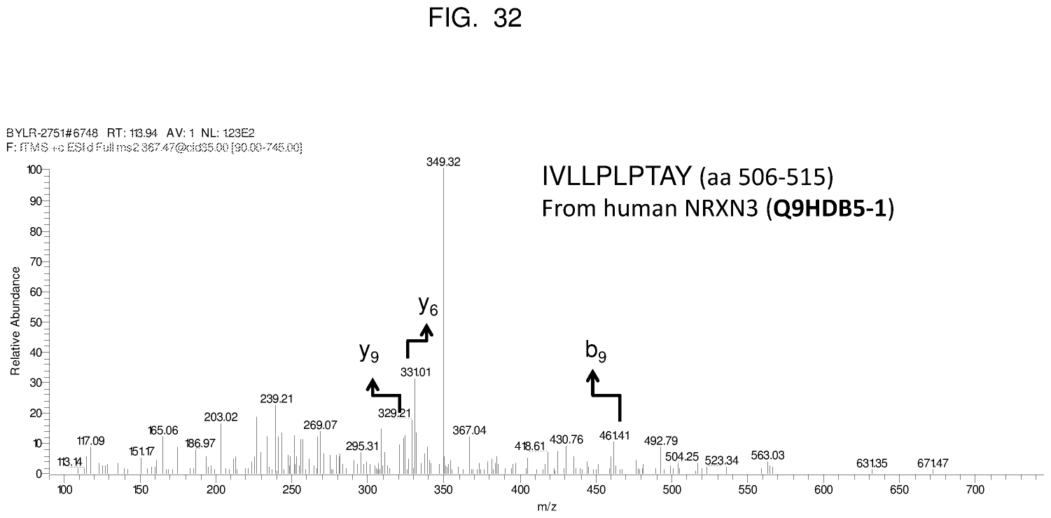

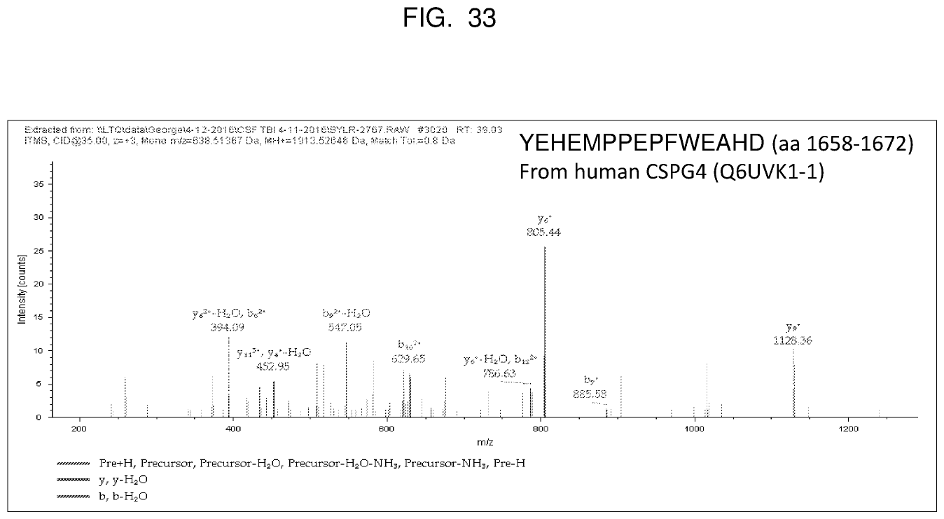

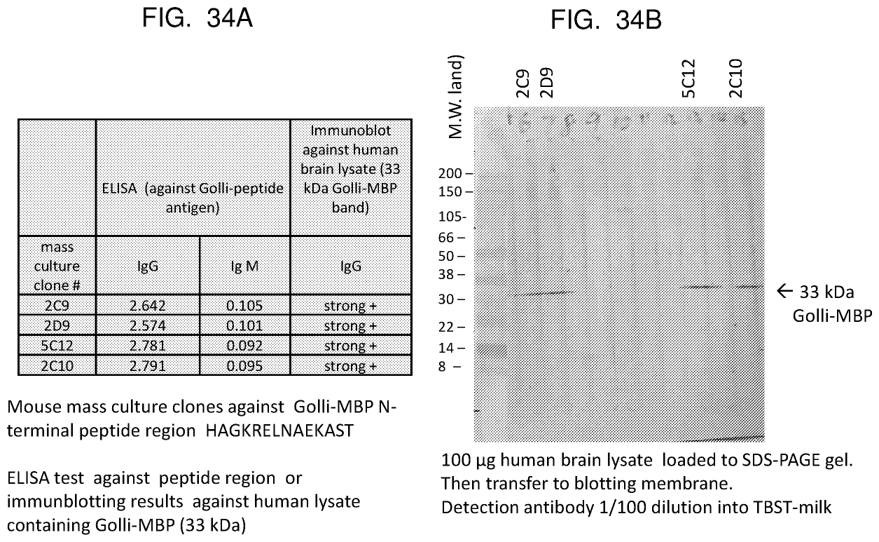

FIG. 13B is an MS/MS spectrum for the shown calpain digestion of humna Tau-441 generated PF with sequence AEPRQEFEVMEDHAGTYG (Aa 2-19 of human Tau-441 (P10636-8) (SEQ ID NO:105). The figure shows the fragment ions for this peptide.

FIG. 13C is an MS/MS spectrum for the sequence of another calpain-produced Tau PF, TLADEVSASLAKQGL (aa 427-441 of Tau-441; SEQ ID NO:138). The figure shows the fragment ions for this peptide.

FIG. 13D is a western blot of the calpain digestion of human tau-441 protein (63K) showing high molecular weight PBP of 40-38K.

FIG. 13E. Top proteolytic peptides of Tau isolated from brain lysate filtrate from TBI-treated human Tau overespressing mouse. Peptides that had the top PSMs value plotted on the y-axis and their corresponding m/z on the x-axis. XCorr value is represented in color with the bar on the right panel as a reference. The brackets at the end of each peptide show adjacent amino acid residue.

FIG. 13F. Schematic representation for the TBI-generated tau peptides recovered from ultrafiltrate fractions as in FIG. 13E. Duplicate peptides found are not shown. None of the peptides shown was found in non-injured control naive samples. Residue # shown on the X-axis. Peptides are ordered from N-terminal to C-terminal.

FIG. 14A provides data showing the identification of a human NRGN PF released into cerebrospinal fluid (CSF) of a human TBI subject with a sequence ILDIPLDDPGANAAAAKIQAS(p)FRGHMARKKIKSGERGRKGPGPGGPGGA (aa 16-64 of human NRGN (NP_006167.1) SEQ ID NO:482). (p) in the sequence indicates phosphorylation modification of the preceding residue.

FIG. 14B shows MS/MS quantification of P-NRGN-BDP in human TBI CSF.

FIG. 14C graphical representation of spectrum of NRGN peptide in human TBI CSF (24 hr).

FIG. 14D shows is a western blot of human NRGN in control CSF and in CSF from a human TBI subject, showing the presence of NGRN and its PBP. For comparison, alpha spectrin and its PBPs are also shown by probing the top part of the blotting membrane with anti-alpha II-spectrin antibody. FIG. 14E is a scatter plot showing densitometric quantitation of control and TBI intact and NGRN PBP. FIG. 14F shows ROC curves of intact NRGN/BDP comparing Control vs. TBI CSF.

FIG. 15A is an MS/MS spectrum of the VIM peptide NVKMALDIEIAT (aa 388-399 of human VIM (P08670; SEQ ID NO:108), charge+2, monoisotopic m/z 699.34711 Da, released into the CSF of a TBI subject. FIG. 15B is an MS/MS spectrum of the VIM PF, LLEGEESRISLPLPNFSSLNSR (aa 403-424; SEQ ID NO:109), released into the CSF of a TBI subject. FIG. 15C shows area under the curve (AUC) for the noted peptides. FIG. 15D is a schematic representation of the noted peptides TBI CSF (24 hr). FIG. 15E is a western blot showing a profile of VIM PBPs (38 kDa and 26 kDa) released into human CSF after TBI. FIG. 15F is a scatterplot of intact VIM and the 38 kDa and 26 kDa VIM PBP released into human CSF after TBI.

FIG. 16A is an MS/MS spectrum of the MBP PF, TQDENPVVHF (aa 107-116, SEQ ID NO:322) derived from human classic MBP, charge+2, monoisotopic m/z 593.96 Da, released into CSF of a human TBI subject. FIG. 16B is a schematic representation of the noted peptides.

FIG. 16C is a western blot providing the profile of MBP breakdown products in human CSF (8000 Da) released less than or equal to 24 hours after TBI, compared to controls (*p<0.01).

FIG. 16D is a scatterplot showing densitometric quantitation of the 8000 Da MBP fragment with mean and SEM. * shows statistical significance over naive (p-value <0.05, 2 tailed unpaired T-test).

FIG. 17 is an MS/MS spectrum of human MBP isoform 2-specific PF, HGSKYLATASTMD (aa 11-24; SEQ ID NO:111), found in a human TBI subject's CSF ultrafiltrate sample.

FIG. 18 is an MS/MS spectrum of human Golli-MBP isoform 1 (304 aa)-specific PF, HAGKRELNAEKASTNSETNRGESEKKRNLGELSRTT (aa 4-39) SEQ ID No. 164).

FIG. 19A is an MS/MS spectrum of GFAP PF (643 aa) ITSAARRSYVSSGEMMVGGLAPGRRLGPGTRLSLARMP (SEQ ID NO:113), found in human TBI subject's CSF sample ultrafiltrate.

FIG. 19B is an MS/MS spectrum of GFAP PF (aa 14-38) YVSSGEMMVGGLAPGRRLGPGTRLS (SEQ ID NO:114), found in human TBI subject's CSF sample ultrafiltrate.

FIG. 19C is an MS/MS spectrum of GFAP PF, DGEVIKES (aa 417-424; SEQ ID NO:115) found in human TBI subject's CSF sample ultrafiltrate.

FIG. 19D is an MS/MS spectrum of GFAP PF, DGEVIKE (aa 417-423; SEQ ID NO:116) found in human TBI subject's CSF sample ultrafiltrate.

FIG. 19E is an MS/MS spectrum of GFAP PF, GEENRITIPVQTFSNLQIRETSLDTKSV (aa 372-399; SEQ ID NO:117) found in a human TBI subject's CSF ultrafiltrate sample.

FIG. 20A is an MS/MS spectrum of Tau-441 PF, AEPRQEFEVMEDHAGTYGLGDRKDQGGYT (aa 2-30; SEQ ID NO:118) identified from a human TBI subject CSF ultrafiltrate sample. FIG. 20B shows sorting data for the noted peptides showing absence in Ctrl and presence in Either Day 1 or 2). The ANOVA/T-test analysis are done based on a datapoint required for all of the replicates (10 control, 5 Day 1 and 7 Day2). FIG. 20C shows a schematic representation for TBI-generated tau proteolytic peptides recovered from CSF ultrafiltrate fractions. Duplicate peptides found are not shown. Peptide amino acid letters are shown on the X-axis. Sequence numbers are shown on the y-axis and are based on human tau-441. None of the peptides shown was found in control CSF samples. Peptides are ordered from N-terminal to C-terminal

FIG. 21 is an MS/MS spectrum for the Calmodulin regulated spectrin-associated protein-1 (CAMSAP-1; #Q5T5Y3-1) PF, SQHGKDPASLLASELVQLH (aa 864-882; SEQ ID NO:119) identified in a human TBI CSF ultrafiltrate sample.

FIG. 22A is an immunoblot showing the presence of CAMSAP1 (177 kDa) and its 110 kDa PBP in human CSF. FIG. 22B is a scatterplot showing both intact CAMSAP1 and the CAMSAP 110 kDa PBP levels are higher in TBI subject CSF compared to control.

FIG. 23 is an MS/MS spectrum for the Calmodulin regulated spectrin-associated protein-3 (CAMSAP-3) PF, LQEKTEQEAAQ (aa 180-190; SEQ ID NO:120) identified in a human TBI CSF ultrafiltrate sample.

FIG. 24 is an MS/MS spectrum for the glutamate decarboxylase 1 (GAD1) PF, HPRFFNQLSTGLDIIGLAG (Q99259-1; aa184-202; SEQ ID NO:121) identified in a human TBI CSF ultrafiltrate sample.

FIG. 25 is an MS/MS spectrum for the Synapsin-1 (SYN1) PF, QDEVKAETIRS (P17600-1; aa 684-694; SEQ ID NO:122), identified in a human TBI CSF ultrafiltrate sample.

FIG. 26 is an MS/MS spectrum for the Synapsin-2 (SYN2) PF, SQSLTNAFSFSESSFFRS (Q9277-1; aa 540-557; SEQ ID NO:123) identified in a human TBI CSF ultrafiltrate sample.

FIG. 27 is an MS/MS spectrum for the Synapsin-3 (SYN3) PF, DWSKYFHGKKVNGEIEIRV (Q14994-1; aa 103-121; SEQ ID NO:124) identified in a human TBI CSF ultrafiltrate sample.

FIG. 28 is an MS/MS spectrum for the Striatin-1 PF, AGLTVANEADSLTYD (043815-1, aa 427-441; SEQ ID NO:125) identified in a human TBI CSF ultrafiltrate sample.

FIG. 29 is an MS/MS spectrum for the growth associated protein 34 (GAP43) PF, AETESATKASTDNSPSSKAEDA (P17677-1; aa 138-159; SEQ ID NO:126) identified in a human TBI CSF ultrafiltrate sample.

FIG. 30A is an MS/MS spectrum for the PF, TKYSEATEHPGAPPQPPPPQQ of human Microtubule-Associated Protein 6 (MAP6; Q96JE9-1; aa 31-51; SEQ ID NO:127) and FIG. 30B is an MS/MS spectrum for the PF, QLPTVSPLPRVMIPTAPHTEYIESS of MAP6 (aa 788-812; SEQ ID NO:128) identified in a human TBI CSF ultrafiltrate sample.

FIG. 31 is an MS/MS spectrum for the Nesprin-1 PF, HSAKEELHR (#Q8NF91; aa 2856-2865; SEQ ID NO:129) identified in a human TBI CSF ultrafiltrate sample.

FIG. 32 is an MS/MS spectrum for the Neurexin-3 PF, IVLLPLPTAY (Q9HDB5-1; aa 506-515; SEQ ID NO:130) identified in a human TBI CSF ultrafiltrate sammple.

FIG. 33 is an MS/MS spectrum for the Chondroitin sulfate proteoglycan 4 (CSPG4) PF, YEHEMPPEPFWEAHD (#Q6UVK1-1; aa 1658-1672; SEQ ID NO:131) identified in a human TBI CSF ultrafiltrate sample.

FIG. 34A is example of mouse mass culture clones against Golli-MBP N-terminal peptide region HAGKRELNAEKAST with ELISA test against this peptide region.

FIG. 34B is the same mass culture clones against Golli-MBP N-terminal peptide region HAGKRELNAEKAST tested with human lysate showing strong detection of Golli-MBP (33 kDa)

### DETAILED DESCRIPTION

### 1. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount. Unless defined otherwise, all technical and scientific terms used herein have the same meaning as commonly understood by one of ordinary skill in the art.

As used herein, the terms “protein breakdown product” or “PBP” refer to a high molecular weight product of protein proteolysis, produced by one or more cleavages of a peptide bonds in the amino acid sequence, i.e., a product of protein cleavage, including chains of any length shorter than the native full-length sequence and longer than about 10,100 Da. The terms “peptide fragment,” or “PF” refer to a low molecular weight products of protein proteolysis, produced by one or more cleavages of a peptide bonds in the amino acid sequence, i.e., a product of protein cleavage. In one example, PFs may include fragments of the intact protein having 85 percent or less the size of the intact protein and greater than 10,000 Da. In another embodiment, PFs may include smaller fragments, i.e. including chains of any length shorter than about 10,000 Da, or 10,100 Da, or such peptide fragments that are able to pass through an ultrafiltration membrane with an approximate 10,000 Da cutoff, including PFs in the range of about 1,000 Da to about 10,000 Da, preferably about 2,000 to 8,000 Da, and most preferably about 2,000 to 5,000 Da. In general, a peptide fragment (PF), as used in this application, refers to an amino acid chain small enough to pass through an ultrafiltration membrane with an approximate 10,000 Da cutoff. As used herein, the term “analyte” and all of its cognates refers to any and all of the proteins, PBPs, or PFs that are analyzed or detected according to this invention.

The PFs and PBPs of the invention are referenced in this application by sequence, amino acid residue number from a protein, or by name. The invention, however, is intended to include peptides that are variants of these particular disclosed sequences. For example, minor differences such as deletion of one or two C- or N-terminal amino acids (or both) of the sequence are contemplated for use with the invention as peptide variants. Other minor differences such a an addition of one or two C- or N-terminal amino acids (or both) of the sequence likewise are contemplated for use with the invention. Minor differences which are caused by variable sequences of the protein, also are contemplated as part of the invention, including differences caused by natural differences in the protein sequence among species or among individuals are intended to be included in certain embodiments of the invention, as well.

As used herein, the phrase “trauma to the central nervous system,” “CNS trauma,” or “traumatic brain injury” includes any sudden injury to the brain, retina, spinal cord, or any part thereof, and includes injury to the projections (e.g., axons, dendrites, neurites) and subcellular parts of cells of the central nervous system due to trauma such as a physical impact or force, or a blast overpressure wave. Examples of CNS trauma include traumatic brain injury (TBI) or traumatic spinal cord injury (SCI). Much of the time, the injury will be the direct result of a traumatic injury, however the invention contemplates uses for injury or destruction of central nervous system tissue and/or cells indirectly caused by trauma, including but not limited to inflammation induced by trauma, swelling induced by trauma, or degenerative disease induced by trauma (such as CTE, Alzheimer's disease, Parkinsonianism, and the like).

As used herein, the term “subject in need” or “subject in need thereof” refers to any animal or a human subject that has been subjected to or suffers from a central nervous system trauma, or is suspected of suffering from a central nervous system injury as a result of trauma.

As used herein, the term “fluid biological sample” refers to a liquid or liquified sample obtained from a subject in need, and includes cerebrospinal fluid, whole blood, plasma, serum, wound fluid, and biopsy or autopsy samples of brain tissue, spinal tissue, retinal tissue, and/or nerves, such as tissue lysates. The samples preferably are prepared for analysis by, for example, centrifugation and/or filtration, preferably by ultrafiltration.

As used herein, in the term “testing a fluid biological sample of the subject for the level” and the term “levels” in the context of test results, “level” refers to the amount or concentration of a target analyte such as a peptide in a fluid biological sample.

As used herein, in the term “anatomical location” refers to a major central nervous system area, such as cortex, hippocampus, striatum, corpus callosum, cerebellum, retina, spinal cord, and the like, but also to cell type such as neuron, glia, astrocyte and the like, and to subcellular regions such as axon, dendrite, extracellular matrix, neuronal nucleus, cortical cytoskeleton and the like.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

### 2. Overview

It was discovered that brain proteins from different central nervous system (CNS) cell types are proteolytically broken down after brain injury into PBP and PF. PBP and PF are released from injured tissue into biofluid, typically cerebrospinal fluid and blood. These proteolytic events are brain injury-mediated and are not found in biofluids of subjects that have not had a traumatic brain injury (TBI).

The present invention identifies a multitude of full-length proteins, PBPs and PFs are produced after traumatic brain injury and released into biological fluids. These compounds can be used to identify specific anatomical regions of the brain and subcellular structures affected, and for diagnostic and prognostic tests. The marker PFs and PBPs are identified from fluid biological samples such as cerebrospinal fluid (CSF), serum, plasma or blood samples. Use of methods such as mass spectrometry identifies unique fragments from proteins damaged from traumatic brain injury.

Unique PBPs and PFs are identified which can locate brain damage to specific brain regions such as the cortex, striatum, white matter and the like. Damage can be linked to brain cell types such as neurons, astrocytes, and oligodendrocytes as well as subcellular structures such as axons, dendrites, growth cones, cortical cytoskeleton, intermediate filaments and extracellular matrix.

Brain-specific or specifically brain-enriched proteins from various CNS cell types (including neuron, astrocyte, oligodendrocytes) and extracellular matrix are released and also are proteolytically broken down into PBPs and PFs of large and small sizes as a result of trauma to the central nervous system and are released from the injured tissue into biofluids, such as cerebrospinal fluid and blood, where they can be measured. Since these proteolytic events are brain injury-mediated, these PBP and PF can be used as injury-specific biomarkers, as well as the proteins. This was supported by the identification in the present application of unique PBPs and PFs. The presence and amount of combinations of these markers allows one to determine the presence of damage or injury to specific brain regions, including the cortex, striatum, and white matter, specific brain cell types such as neurons, astrocytes, and oligodendrocytes, and specific subcellular structures, including axons, dendrites, growth cones, cortical cytoskeleton, intermediate filaments and extracellular matrix.

Methods of the invention involve testing fluid biological samples from a subject, such as a mouse traumatic brain injury model or a human central nervous system trauma subject. The sample is subjected to ultrafiltration with a low molecular weight (10,000 Da) cutoff membrane to separate the smaller PFs from the larger PBPs and proteins, then the resulting fractions are subjected to testing to identify specific peptides in the filtrate and the larger peptides and proteins in the retentate. Testing can include a tandem mass spectrometry proteomic method and/or immunological methods such as high sensitivity immunoblotting. Time course measurements of post-injury biofluid levels of these proteins, PBPs, or PFs can be used as TBI and CNS injury diagnostic and prognostic tools at different time periods post-injury when compared to levels as recovery progresses and in normal controls.

### 3. Embodiments of the Invention

### A. Introduction

A biomarker as defined by the National Academy of Sciences, and as used herein, the presence of which indicates or signals one or more events in biological samples or systems. Biomarkers for central nervous system injury are valuable and unbiased tools in defining the severity of CNS injury because they reflect the extent of brain and spinal cord damage in emergency medicine, neurointensive care and hospitalization settings. The invention therefore includes a fast turn around point-of-care diagnostic biofluid test and device for deployment in various hospital settings. A small amount of subjects' blood samples can be used on the device and levels of specific combination of two or more of the biomarker PFs can be determined.

Generally, the higher the levels of these biomarker levels, the more severe the injury. For example, in an emergency medicine setting, the more severe brain or spinal cord injury subjects can then be admitted to hospital for treatment and monitoring while the mildly injured subjects can be released. Thus the biomarkers of the invention can be used as triaging tools. For subjects already in a neurointensive care unit, unresolved high biofluid levels of CNS biomarkers or further elevations of such biomarkers can indicate the deterioration of the subject's condition or the evolution of the injury. Thus aggressive medical interventions (such as surgery or other procedures or treatments) might be administrated. The PBP and PF biomarkers can be used for monitoring and management of critically injured subjects. For those TBI or spinal cord injury patients who are moderately injured and are staying in hospital, periodic monitoring of their biofluid levels of CNS biomarkers can be useful to detect delayed elevations of the biomarkers, which could indicate occurrence of a secondary injury or the deterioration or evolution of the initially moderate injury to a more severe condition, or development of post-trauma neurodegeneration, allowing more aggressive medical monitoring or medical intervention to be administered. CNS injury biomarkers in the acute or subacute phase can inform on and/or improve neurological recovery or patient outcome. This information can be very useful for patient or caretaker in terms of future care planning, personal life decision-making and arrangement of rehabilitation.

Some metabolite candidates such as N-acetyl aspartate (NAA, a neuronal/axonal marker), creatine (gliosis marker), and choline (indicator of cellular turnover related to both membrane synthesis and degradation) can be used as biomarkers for monitoring the pathobiological changes of primary and secondary damage in TBI using proton magnetic resonance spectroscopy (1H-MRS). In vivo 1H-MRS is a valuable tool for noninvasive monitoring of brain biochemistry by quantifying the changes in the metabolites in brain tissue. However, due to the relatively small size of the spinal cord and magnetic susceptibility effects from the surrounding bony structures, acquiring MR spectra with adequate signal to-noise ratio (SNR) is difficult, and does not allow detection of subtle changes in metabolite levels.

Proteomic analysis is a technique for simultaneously detecting multiple proteins in a biological system. It provides robust methods to study protein abundance, expression patterns, interactions, and subcellular localization in blood, organelle, cell, tissue, organ or organism to provide accurate and comprehensive data about that system. For example, proteomics can use extensive sample procedures and data-dependent acquisition to follow disease-specific proteins (identity and concentration). It facilitates the identification of all differentially expressed proteins at any given time in a proteome (the entire complement of proteins that can be expressed by a cell, tissue, or organism) and correlates and compares these patterns with those in a healthy system during disease progression. Proteomics has been used to study protein expression at the molecular level with a dynamic perspective that helps to understand the mechanisms of the disease.

The complexity, immense size and variability of the neuroproteome and the extensive protein—protein and protein—lipid interactions limit the ability of mass spectrometry to detect all peptides/proteins contained within the sample. Further, some peptides/proteins are extraordinarily resistant to isolation. Therefore, the analytical methods for the separation and identification of peptides/proteins must manage all of these issues. This invention addresses these problems by using separation techniques combined with powerful new mass spectrometry technologies to expand the scope of protein identification, quantitation and characterization.

The complexity of a biological sample can be reduced by separation or fractionation at the protein or peptide level. Multidimensional liquid chromatography (LC) was used in two or more different types of sequential combinations to significantly improve the resolution power and resulted in a large number of proteins being identified. Any of these methods are contemplated for use with the invention.

Ion-exchange chromatography (IEC) in the first dimension was very suitable for the separation of proteins and PFs by separating proteins based on their differences in overall charges. IEC's stationary phase is either an anion or a cation exchanger, prepared by immobilization of positively or negatively charged functional groups on the surface of chromatographic media, respectively. Protein or peptide separation occurs by linear change of the mobile-phase composition (salt concentration or pH) that decreases the interactions of proteins with the stationary phase, resulting in finally eluting the proteins. SDS-PAGE can be used for further protein separation by apparent molecular weight with the resolving distance optimized for the proteome of interest. PFs can be separated by their hydrophobicity using a reversed phase C18 column directly coupled to the electrospray mass spectrometer (ESI-LC-MS/MS). Reversed-phase liquid chromatography (RPLC) is most often used in the second dimension due to its compatibility with downstream mass spectrometry (sample concentration, desalting properties, and volatile solvents).

Mass spectrometry (MS) also is an important tool for protein identification and characterization in proteomics due to the high selectivity and sensitivity of the analysis and can be used in the invention. Electrospray ionization (ESI) is considered a preferred ionization source for protein analysis due to two characteristics: first, the ability to produce multiply-charged ions from large molecules (producing ions of lower m/z that are readily separated by mass analyzers such as quadrupoles and ion traps), and second, the ease of interfacing with chromatographic liquid-phase separation techniques. Electrospray ionization followed by tandem mass spectrometry (ESI-MS/MS) is one of the most commonly used approaches for protein identification and sequence analysis.

This invention takes advantage of proteomic analysis to identify biomarkers in complex biological samples, for example biofluids, to diagnose CNS traumatic injury in a subject, to assess the severity and location of the traumatic injury, and to make a determination of prognosis for the subject. The subject preferably is a human or other mammal, for example a laboratory animal, farm animal, companion animal, zoo animal, or most preferably is a rodent or primate, including a human subject or patient. The mammals contemplated as subjects with respect to this invention include rats, mice, ferrets, swine, monkeys, and primates, including humans.

### B. Subjects and Sampling

The injuries contemplated for diagnosis, determination of severity and location, or prognosis include any injury to the central nervous system, of whatever cause. Injuries to the peripheral nerves also are included and are contemplated with respect to this invention. The injury includes injury to the brain, retina, and/or spinal cord, or the peripheral or cranial nerves, and may be localized to a particular physical area or may be generalized. Injuries can be caused by direct trauma, or by inflammation or swelling and edema, contusion, diffuse axonal injury, cerebrovascular injury, hypoxia or anoxia, ischemia, a thromboembolic event, cerebrovascular occlusion or other acute or chronic circulatory disorder, toxins or poisons, envenomation, hemorrhage or hypovolemia, and the like, which cause a physical trauma, directly or indirectly, to the central nervous system. Thus, the subjects referred to herein are any mammal that either suffers from or is suspected of suffering from an injury as discussed above.

The samples that can be usefully collected and tested for protein breakdown products according to the invention include fluid biological samples such as cerebrospinal fluid, whole blood, plasma, serum, and the like, or biopsy, autopsy or necropsy CNS lysate samples and other fluid samples. These samples are collected from the subject according to methods known in the art.

Samples are collected from the subject after an injury to the central nervous system, or an incident that indicates such an injury may have occurred. Incidents such as physical and direct trauma to the head or spine (i.e., sports injury, surgery, vehicular accident, falls, and the like) and its sequelae, illness (i.e., tumor, encephalitis, and the like), or hypoxia (i.e., near drowning, myocardial infarction, embolism, and the like), are specifically contemplated, but are not intended to be limited. The person of skill in the art, such as physician or trauma specialist can easily determine if an injury to the central nervous system is present or should be suspected. Preferably, a sample for diagnostic purposes is collected up to 24 hours after initial injury or up to 3 days (72 hours) after initial injury.

The initial samples can be collected immediately or within about 72 hours after trauma occurs or after injury is suspected, preferably within about 24 hours or one day, and can include one sample only or multiple samples (such as two or more of CSF and blood, serum, brain biopsy, and the like). Further, a second or more than one subsequent sample(s) can be collected at one or several additional subsequent times. For example, samples can be collected hourly, twice daily, daily, every two days, weekly, monthly, or any convenient interval for a period of time deemed to be necessary based on the condition of the patient. A suitable time for continued testing can include two days, a week, two weeks, a month, two months, six months, a year, several years, or for the remainder of a patient's lifetime.

An advantage to collecting multiple samples over a time course (for example, over a week, month, several months, years or longer) is that it allows the practitioner to compare the number, type, and amount of protein breakdown products appearing in the samples over time, to assist in determining the course of the injury or the progress of the subject or patient. Repeated sampling allows the practitioner to determine if peptide levels are diminishing or remaining elevated, thus determining whether the injury to the central nervous system is improving, becoming chronic, or becoming more severe over a course of time.

### C. Protein Breakdown Products and Peptide Fragments

Intact proteins such as calcium binding protein S100 beta (S100β), glial fibrillary acidic protein (GFAP), myelin basic protein (MBP), neuron specific enolase (NSE), neurofilament protein (NFL), SBDP150/SBDP145/SBDP120, ubiquitin C-terminal hydrolase-L1 (UCH-L1) and microtubule-associated 2 (MAP-2) have been identified as potential markers of brain damage. However, due to the complexity of TBI and other central nervous system injury, multiple interventions that target the different complications of the injury may be required in a clinical setting. Previous methods using a single biomarker are unlikely to be successful for either diagnostic or prognostic purposes in human patients. Therefore, although the sample or samples can be tested for only one of the biomarkers disclosed here as part of the invention, it is preferable to test for more than one in each sample. Preferred PFs according to the invention are provided in Table 1, below. In preferred methods, one or two PFs from each protein in the table are tested in each sample. In other embodiments, proteins, PBPs, and/or PFs from each category are analyzed.

TABLE 1
Preferred Peptide Fragment Biomarkers

| Protein Name, | | | | |
|---|---|---|---|---|
| Source, | | | | |
| Uniprot | | | | |
| number, and | | | | |
| amino acid | | | | SEQ ID |
| residues | Peptide Sequence | Injury Indicated | Peptide Name | NO |
| Human Tau-441, (isoform 2; isoform | | | | |
| Tau-441, Tau 4); P10636-8 | | | | |
| 2-21 | AEPRQEFEVMEDHA | Axonal injury, | “Tau-441 N- | 132 |
| | GTYGLG | neurodegeneration | terminal peptide 1” | |
| 90-123 | AAQPHTEIPEGTTAE | Axonal injury, | “Tau-441 N- | 133 |
| | EAGIGDTPSLEDEAA | neurodegeneration | terminal peptide 2” | |
| | GHVTQARMVS | | | |
| 311-323 | KPVDLSKVTSKCG | Axonal injury, | “Tau-441 center | 134 |
| | | neurodegeneration | peptide 1” | |
| 315-326 | LSKVTSKCGSLG | Axonal injury, | “Tau-441 center | 135 |
| | | neurodegeneration | peptide 2” | |
| 379-403 | RENAKAKTDHGAEI | Axonal injury, | “Tau-441 | 136 |
| | VYKSPVVSGDT | neurodegeneration | center peptide 3” | |
| 404-426 | SPRHLSNVSSTGSID | Axonal injury, | “Tau-441 C- | 137 |
| | MVDSPQLA | neurodegeneration | terminal peptide 1” | |
| 427-441 | TLADEVSASLAKQGL | Axonal injury, | “Tau-441 C- | 138 |
| | | neurodegeneration | terminal peptide 2” | |
| 434-441 | ASLAKQGL | Axonal injury, | “Tau-441 C- | 139 |
| | | neurodegeneration | terminal peptide 3” | |
| | Phosphorylated Human Tau-441, (isoform 2; isoform Tau-441, Tau 4); | | | |
| | P10636-8 | | | |
| 395-412 | KSPVVSGDTSPRHLS | Axonal injury, | “Phospho-Tau-441 | 140 |
| 3xPhospho- | NVS | neurodegeneration | C-terminal peptide | |
| sites | | | 1” | |
| S396(100); | | | | |
| S400(100); | | | | |
| S404(88.9) | | | | |
| 395-426 | KSPVVSGDTSPRHLS | Axonal injury, | “Phospho-Tau-441 | 141 |
| 3xPhospho | NVSSTGSIDMVDSP | neurodegeneration | C-terminal peptide | |
| S396(99) | QLA | | 2” | |
| 413-426 | STGSIDMVDSPQLA | Axonal injury, | “Phospho-Tau-441 | 142 |
| 1xPhospho- | | neurodegeneration | C-terminal peptide | |
| site | | | 3” | |
| S416(99.9) | | | | |
| | Human Tau-758 (isoform 1, isoform PNS-Tau, PHF-Tau), P10636-1 | | | |
| 372-401 | PQLKARMVSKSKDGTG | Axonal injury, | “Tau-758 specific | 143 |
| | SDDKKAKTSTRSSA | neurodegeneration | center peptide 1” | |
| 411-457 | SPKHPTPGSSDPLIQPSS | Axonal injury, | “Tau-758 specific | 144 |
| | PAVCPEPPSSPKYVSSV | neurodegeneration | center peptide 2” | |
| | TSRTGSSGAKEM | | | |
| 435-486 | PPSSPKYVSSVTSRTGSS | Axonal injury, | “Tau-758 specific | 145 |
| | GAKEMKLKGADGKTKI | neurodegeneration | center peptide 3” | |
| | ATPRGAA | | | |
| 444-465 | SVTSRTGSSGAKEMKL | Axonal injury, | “Tau-758 specific | 146 |
| | KGADGK | neurodegeneration | peptide” | |
| 411-434 | SPKHPTPGSSDPLIQPSS | Axonal injury, | “Tau-758 specific | 147 |
| | PAVCPE | neurodegeneration | center peptide 2” | |
| 435-459 | PPSSPKYVSSVTSRTGSS | Axonal injury, | “Tau-758 specific | 148 |
| | GAKEMKL | neurodegeneration | center peptide 3” | |
| Human Neurogranin Q92686 | | | | |
| 18-64 | ILDIPLDDPGANAAAAK | Synaptic Injury | “Neurogranin | 149 |
| | IQASFRGHMARKKIKSG | | Peptide 1” | |
| | ERGRKGPGPGGPGGA | | | |
| 18-64 | ILDIPLDDPGANAAAAK | Synaptic Injury | “Neurogranin | 150 |
| | IQAS(p)FRGHMARKKIK | | Peptide 2 (Ser-36 | |
| | SGERGRKGPGPGGPGGA | | phosphorylated)” | |
| 13-38 | DDDILDIPLDDPGANAA | Synaptic Injury | “Neurogranin | 151 |
| | AAKIQASFR | | Peptide 3” | |
| 13-38 | DDDILDIPLDDPGANAA | Synaptic Injury | “Neurogranin | 152 |
| | AAKIQAS(p)FR | | Peptide 4 (Ser-36 | |
| | | | phosphorylated)” | |
| 24-65 | PGANAAAAKIQASFRG | Synaptic Injury | “Neurogranin | 153 |
| | HMARKKIKSGERGRKG | | Peptide 5” | |
| | PGPGG | | | |
| 24-65 | PGANAAAAKIQAS(p)FR | Synaptic Injury | “Neurogranin | 154 |
| | GHMARKKIKSGERGRK | | Peptide 6 (Ser-36 | |
| | GPGPGG | | phosphorylated)” | |
| Human Vimentin P08670 | | Astroglial Injury | | |
| 388-399 | NVKMALDIEIAT | Astroglial Injury | “Vimentin C- | 155 |
| | | | terminal peptide 1” | |
| 403-424 | LLEGEESRISLPLPNFSS | Astroglial Injury | “Vimentin C- | 156 |
| | LNLR | | terminal peptide 2” | |
| 388-465 | NVKMALDIEIATYRKLL | Astroglial Injury | “Vimentin C- | 157 |
| | EGEESRISLPLPNFSSLN | | terminal peptide 3” | |
| | LRETNLDSLPLVDTHSK | | | |
| | RTLLIKTVETRDGQVIN | | | |
| 1-75 | MSTRSVSSSS | Astroglial Injury | “Vimentin N- | 492 |
| | YRRMFGGPGT | | terminal peptide 1” | |
| | ASRPSSSRSY | | | |
| | VTTSTRTYSL | | | |
| | GSALRPSTSR | | | |
| | SLYASSPGGV | | | |
| | YATRSSAVRL RSSVP | | | |
| 2-47 | STRSVSSSSYRRMFGGP | Astroglial Injury | “Vimentin N- | 159 |
| | GTASRPSSSRSYVTTST | | terminal peptide 2” | |
| | RTYSLGSALR | | | |
| Human MBP Isoform 5 P02686-5 | | Myelin damage/ | | |
| | | oligodendrocyte | | |
| | | injury | | |
| 11-24 | HGSKYLATASTMD | Myelin damage/ | “MBP N-terminal | 160 |
| | | oligodendrocyte | peptide 1” | |
| | | injury | | |
| 11-43 | HGSKYLATASTMDHAR | Myelin damage/ | “MBP N-terminal | 161 |
| | HGFLPRHRDTGILDSIG | oligodendrocyte | peptide 2” | |
| | R | injury | | |
| 105-140 | GRTQDENPVVHFFKNI | Myelin damage/ | “MBP center | 162 |
| | VTPRTPPPSQGKGRGLS | oligodendrocyte | peptide” | |
| | LSRF | injury | | |
| 165-178 | HKGFKGVDAQGTLS | Myelin damage/ | “MBP C-terminal | 163 |
| | | oligodendrocyte | peptide” | |
| | | injury | | |
| Human Golli-MBP1 P02686-1 | | Myelin damage/ | | |
| | | oligodendrocyte | | |
| | | injury | | |
| 4-38 | HAGKRELNAEKASTNS | Myelin damage/ | “Golli-MBP1 | 164 |
| | ETNRGESEKKRNLGELS | oligodendrocyte | isoform-specific N- | |
| | RTT | injury | terminal peptide” | |
| 75-116 | NAWQDAHPADPGSRPH | Myelin damage/ | “Golli-MBP1 | 165 |
| | LIRLFSRDAPGREDNTF | oligodendrocyte | isoform-specific | |
| | KDRPSESDE | injury | center peptide” | |
| Human Glial Fibrillary Acidic protein | | Astroglial injury | | |
| P14136-1 | | | | |
| 6-43 | ITSAARRSYVSSGEMM | Astroglial injury | “GFAP N-terminal | 166 |
| | VGGLAPGRRLGPGTRL | | peptide 1” | |
| | SLARMP | | | |
| 14-38 | YVSSGEMMVGGLAPG | Astroglial injury | “GFAP N-terminal | 167 |
| | RRLGPGTRLS | | peptide 2” | |
| 12-33 | RSYVSSGEMMVGGLAP | Astroglial injury | “GFAP N-terminal | 168 |
| | GRRLGP | | peptide 3” | |
| 9-49 | AARRSYVSSGEMMVG | Astroglial injury | “GFAP N-terminal | 169 |
| | GLAPGRRLGPGTRLSLA | | peptide 4” | |
| | RMPPPLPTR | | | |
| 11-30 | ARRSYVSSGEMMVGGL | Astroglial injury | “GFAP N-terminal | 170 |
| | APGRR | | peptide 5” | |
| 26-45 | APGRRLGPGTRLSLAR | Astroglial injury | “GFAP N-terminal | 171 |
| | MPP | | peptide 5” | |
| 372-399 | GEENRITIPVQTFSNLQI | Astroglial injury | “GFAP C-terminal | 172 |
| | RETSLDTKSV | | peptide 1” | |
| 382-416 | QTFSNLQIRETSLDTKS | Astroglial injury | “GFAP C-terminal | 173 |
| | VSEGHLKRNIVVKTVE | | peptide 2” | |
| | MR | | | |
| 417-423 | DGEVIKES | Astroglial injury | “GFAP C-terminal | 174 |
| | | | peptide 3” | |
| 417-422 | DGEVIKE | Astroglial injury | “GFAP C-terminal | 175 |
| | | | peptide 4” | |
| 417-332 | DGEVIKESKQEHKDVM | Astroglial injury | “GFAP C-terminal | 176 |
| | | | peptide 5” | |
| 384-400 | FSNLQIRETSLDTKSVSE | Astroglial injury | “GFAP C-terminal | 177 |
| | | | peptide 6” | |
| 417-423 | DGEVIKESK | Astroglial injury | “GFAP C-terminal | 178 |
| | | | peptide 7” | |

Microtubule-associated Protein 6
(MAP6) Q7TSJ2

| | | | | |
|---|---|---|---|---|
| 31-51 | TKYSEATEHPGAPP | Astroglial injury | “MAP6 N-terminal | 179 |
| | QPPPPQQ | | peptide 1” | |
| 788-812 | QLPTVSPLPRVMIPT | Astroglial injury | “MAP6 C-terminal | 180 |
| | APHTEYIESS | | peptide 1” | |

Synapsin I (SYNI) P17600-1 or P17600-2

| | | | |
|---|---|---|---|
| 684-694 | QDEVKAETIRS | Pre-synaptic terminal | 181 |
| | | injury | |

Synapsin II (SYN2) Q9277-1 or Q64332

| | | | |
|---|---|---|---|
| 540-557 | SQSLTNAFSFSESSFF | Pre-synaptic terminal | 182 |
| | RS | injury | |

Synapsin III (SYN3) Q14994-1 or P07437

| | | | |
|---|---|---|---|
| 540-557 | DWSKYFHGKKVNG | Pre-synaptic terminal | 183 |
| | EIEIRV | injury | |
| 359-391 | GEHVEEDRQLmADL | Pre-synaptic terminal | 184 |
| | VVS | injury | |

Complement C1q subcomponent subunit B D6R934

| | | |
|---|---|---|
| HGEFGEKGDPGIPG | Microglia activation | 701 |

Complement C3 P01024

| | | |
|---|---|---|
| HWESASLL | Microglia activation | 702 |
| VKVFSLAVNLIAI | Microglia activation | 703 |

Complement receptor type 1 CR1 E9PDY4

| | | |
|---|---|---|
| KTPEQFPFAS | Microglia activation | 704 |

Complement C5 P01031

| | | |
|---|---|---|
| VTcTNAELVKGRQ | Microglia activation | 705 |

Complement C1s P09871

| | | |
|---|---|---|
| IISGDTEEGRLcGQR | Microglia activation | 706 |
| SSNNPHSPIVE | | |

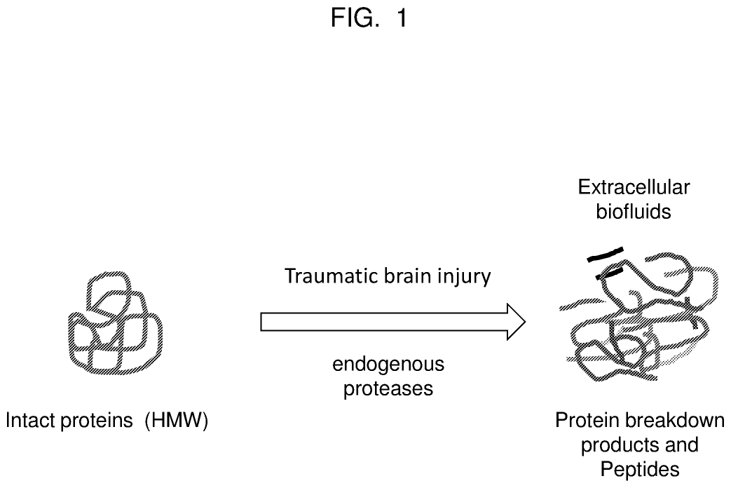

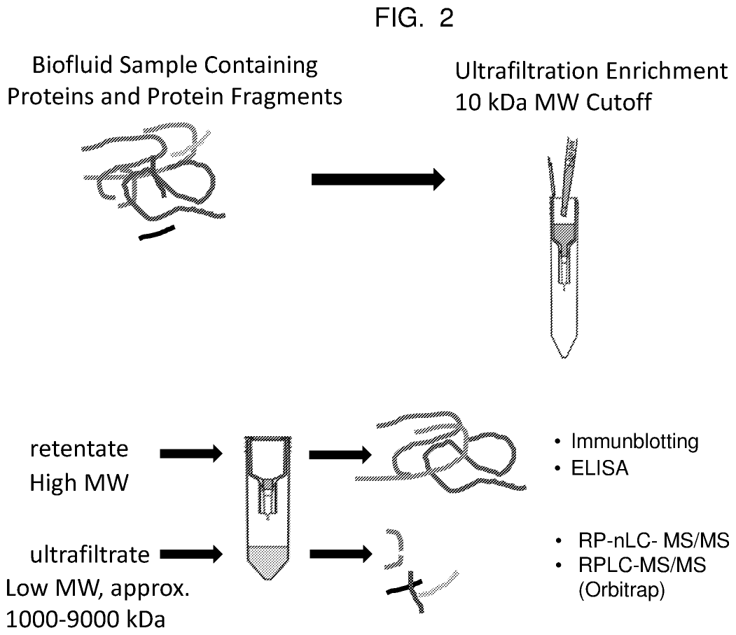

The above table shows novel CNS traumatic injury biomarkers identified as PFs derived from CNS proteins due to traumatic injury activated proteolysis, in accordance with the schematic diagram in FIG. 1. These PFs include those derived from these brain proteins: human Tau-441 (isoform 2; isoform Tau-441, Tau 4; P10636-8), human Tau-758 (isoform 1, isoform PNS-Tau, PHF-Tau, P10636-1), human NRGN (Q92686), human VIM (P08670), Human MBP Isoform 5 (P02686-5), human Golli-MBP1 (P02686-1), human Glial Fibrillary Acidic protein (GFAP; P14136-1), Microtubule-associated protein 6 (MAP6; Q7TSJ2), human Synapsin I (SYNI) (P17600-1 or P17600-2), Synapsin II (SYN2) (Q9277-1), Synapsin III (SYN3) (Q14994-1), human complment proteins (Clq (D6R934), C3 (P01024), C5 (P01031), Cls (P09871) and complment receptor CR1 (Complement receptor 1; E9PDY4), CR1 (Complement receptor-2; P20023) C1QRF (Clq-related factor; 075973). As shown in the workflow chart in FIG. 2, during the brain protein proteolysis process after traumatic injury to CNS, low molecular weight PFs are formed, often paralleled by formation of high molecular weight PBP. Thus, brain proteins from different central nervous system (CNS) areas and cell types are proteolytically broken down after brain injury into PBPs and PFs, which are subsequently released from injured tissue into biofluid, typically cerebrospinal fluid and blood. These proteolytic events are brain injury-mediated and are not found in biofluids of subjects that have not had a traumatic brain injury (TBI) or spinal cord injury.

Additional TBI proteolytic biomarker PBPs or PFs were also derived from brain proteins Synapsin-I, II, III (SYN1, SYN2, SYN3), Cortexin-1,2,3 (CTXN1, CTXN2, CTXN3), Striatin (STRN), NRGN (fragment), MBPS (fragment) Golli-MBP1, VIM, Brain acidic soluble protein (BASP1, BASP2 (GAP33)), Neurochondrin, Nesprin-1 Glutamate Decarboxylase-1, 2 (GAD1, GAD2), Neurexin-1, 2, 3 (NRXN1, NRXN2, NRXN3) Calmodulin-binding spectrin associated proteins-1, 2, 3 (CAMSAP1, 2, 3), and Chondroitin sulfate proteoglycans (CSPG4, Neurocan (CSPG3) and brevican. These proteins are listed in Table 2, below, showing the brain area in which they are located, and therefore the brain area which is associated with the appearance of the biomarker(s) upon injury. Thus, to determine if an injury to astroglia, for example, is to be diagnosed or investigated, VIM-derived PFs should be analyzed; if an injury to neuron cell bodies is to be diagnosed or investigated, BASP1 and neurochondrin derived PFs should be analyzed.

TABLE 2
Brain Proteins with PBP or PF Released after Traumatic CNS
Injury and their Associated Brain Area

| | Signifying Injury to Brain |
|---|---|
| Protein | Area/Location |
| Cortex-1 | cortex |
| Neurochondrin | Neuron cell body |
| Neurogranin | post-synaptic |
| Synapsin LII, III | presynaptic |
| Striatin | striatum |
| Vimentin | astroglia |
| MBP (classic) | white matter/oligodendrocyte |
| Golli-MBP | white matter/oligodendrocyte |
| GAD1, GAD2 | neuron cell body |
| Chondroitin sulfate proteoglycan 4 | extracellular matrix |
| Neurocan | extracellular matrix |
| Brevican | extracellular matrix |
| CAMSAP1,2,3 | cortical cytoskeleton/axon |
| Neurexin-1, -2, -3 | presynaptic |
| BASP1 | neuron cell body |
| GAP43/BASP2 | neuron growth cone |
| Nesprin-1 | neuron nucleus |
| MAP6 | dendrite |
| Tau-441 isoform (Tau isoform 2, | axon |
| Tau-F; P10636-8) | |
| Tau-758 (isoform 1, PHF-Tau; | axon |
| P10636-1 | |

The above table provides proteins or proteolytic PFs released after traumatic injury to the CNS (e.g. TBI) and their associated brain region, brain cell type or neuronal subcellular location. The work presented here used an in vitro brain injury model with mouse brain lysate and purified brain protein incubation with calcium solution or protease calpain, an in vivo mouse traumatic brain injury model and human traumatic brain injury biofluid (cerebrospinal fluid or CSF) samples. These samples were analyzed using separation by ultrafiltration with low a molecular cutoff filter, a tandem mass spectrometry proteomic method and immunological methods including high sensitivity immunoblotting to detect and identify a number of brain-specific or brain-enriched proteins from various CNS cell types (neurons, astrocytes, oligodendrocytes) or extracellular matrix. Proteins in the central nervous system are proteolytically broken down into PBPs and PFs upon injury to the tissues. The PBPs and PFs are released from injured tissue into biofluids (such as cerebrospinal fluid and blood) and can be detected there as shown above. Since these proteolytic events are brain injury-mediated, the PBPs and PFs were identified to be injury-specific biomarkers.

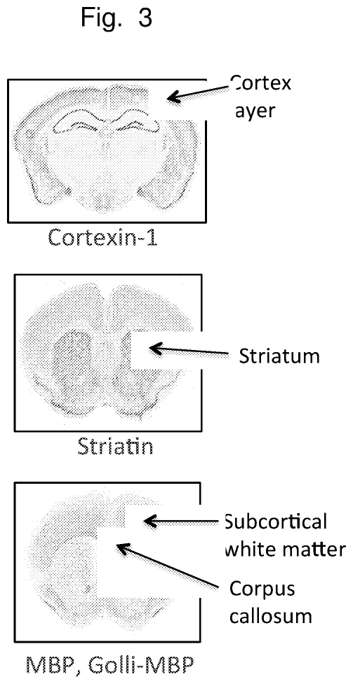

FIG. 3 shows the brain anatomical localization of brain proteins myelin basic protein, striatin and cortexin-1 (based on mRNA abundance of the proteins) are enriched in the subcortical white matter, striatum and cortex layer respectively. Other brain cell type specific markers identified here include PFs of VIM, GFAP, MRC1, Golli-MBP, BASP1, neurochrondin, calmodulin-regulated spectrin-associated proteins (CAMSAP 1, CAMSAP 2 and CAMSAP 3), synapsin 1, synapsin 2, synapsin 3, neurexin, NRGN, CAMPK-II, nesprin-1, chondroitin sulfate proteoglycan 4 (CSPG4), neurocan, and brevican.

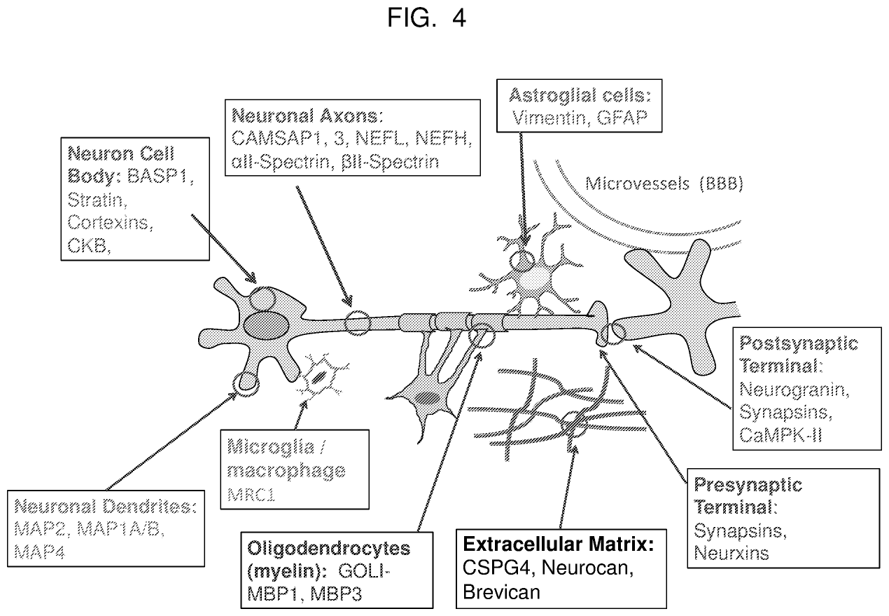

FIG. 4 shows the extracellular, cellular and subcellular locations of brain protein-derived PBP sources that can serve as informative biomarkers for brain injury. This reinforces the utility of informing a practitioner of the specific brain regions (e.g., cortex, striatum), brain cell types (e.g., neuron, astrocyte, oligodendrocyte), subcellular structures (axon, dendrites, growth cone, cortical cytoskeleton, intermediate filament) and extracellular matrix that might be injured or damaged by testing for the indicated PFs formed by injury to that area.

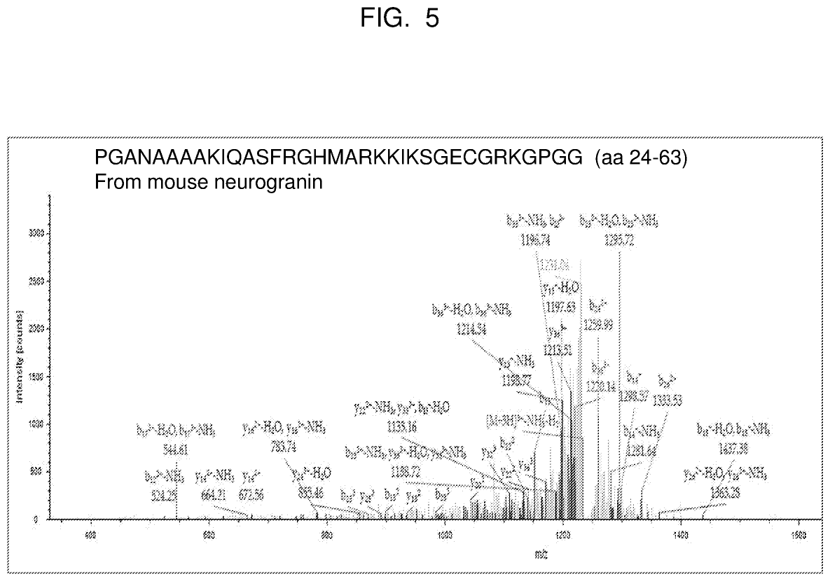

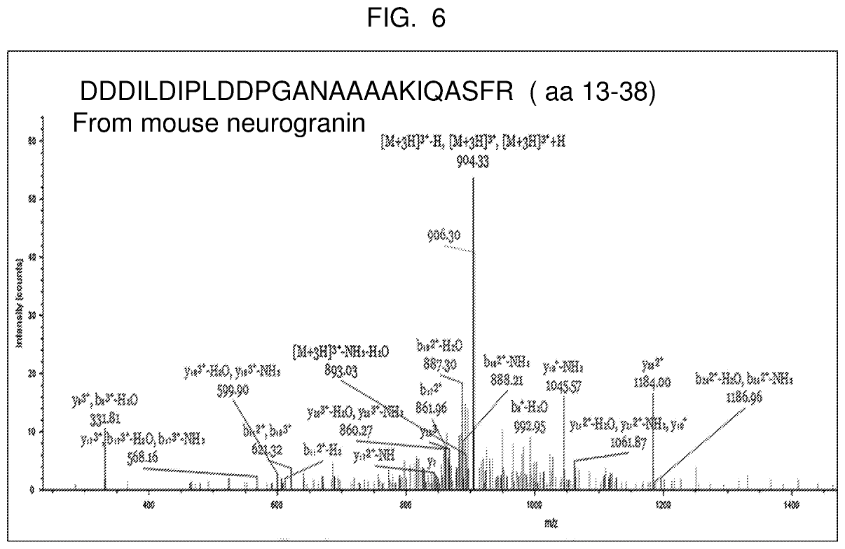

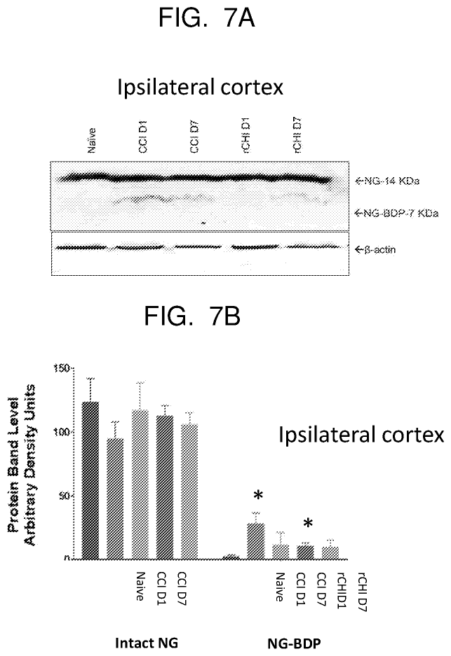

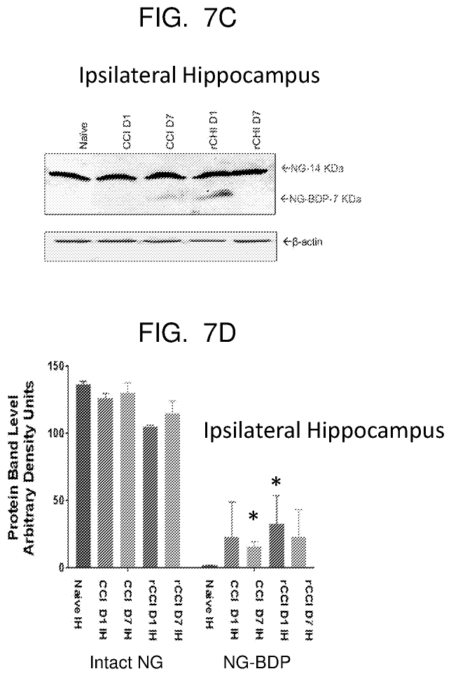

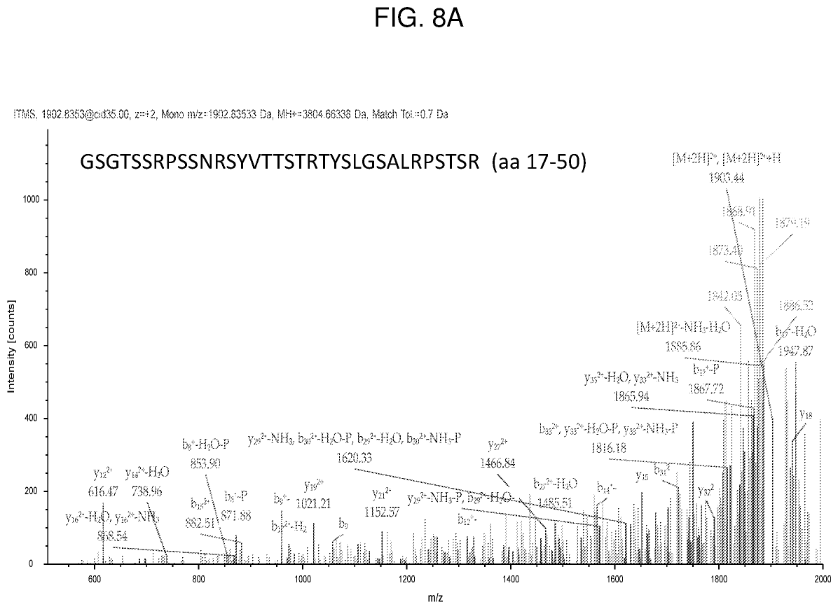

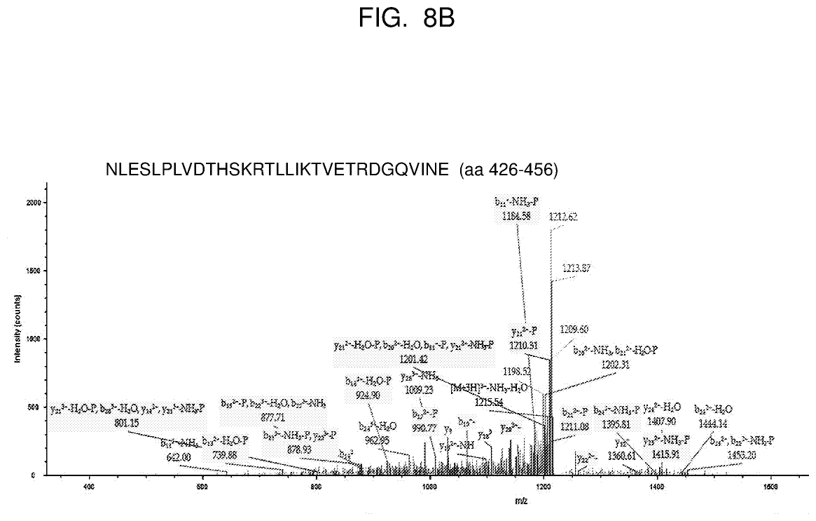

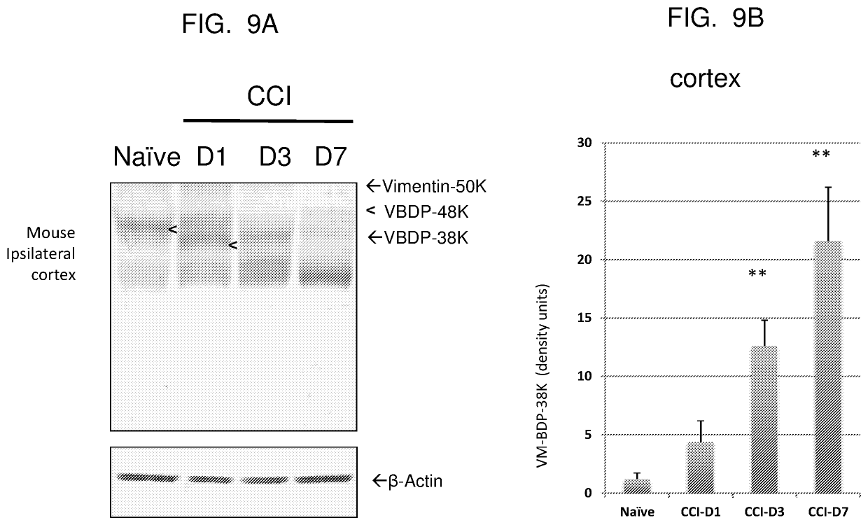

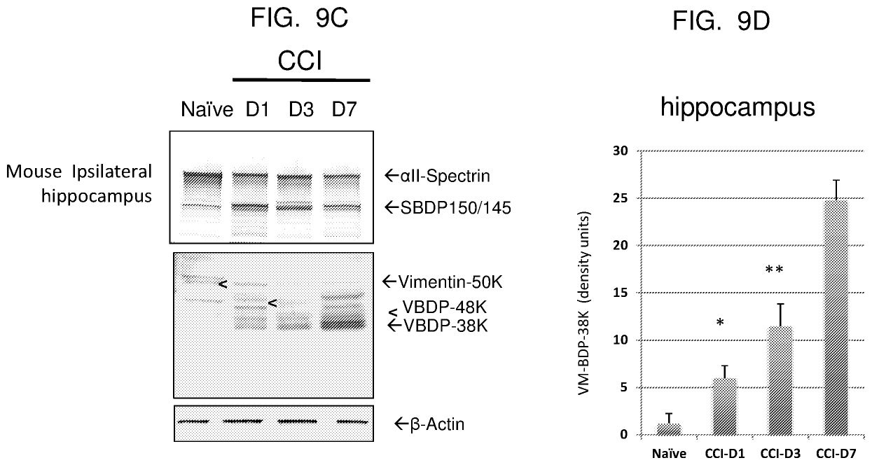

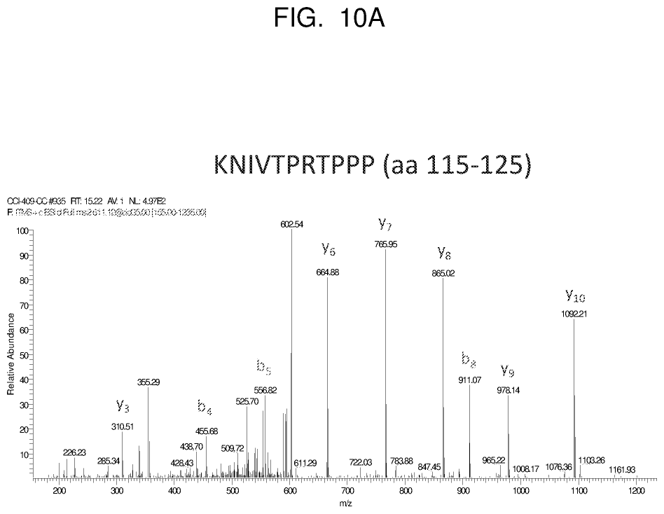

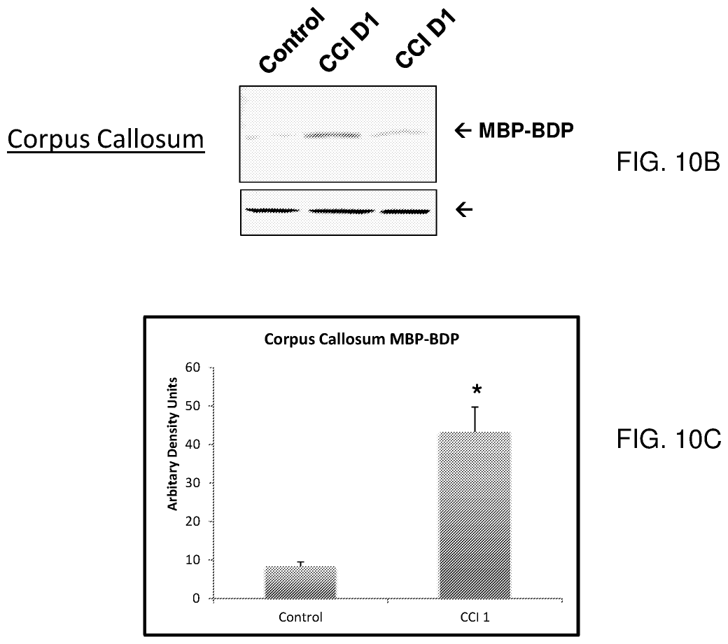

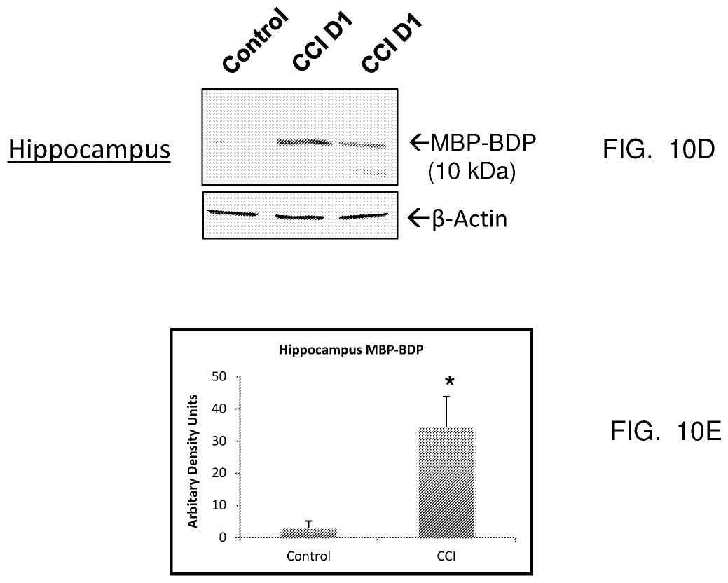

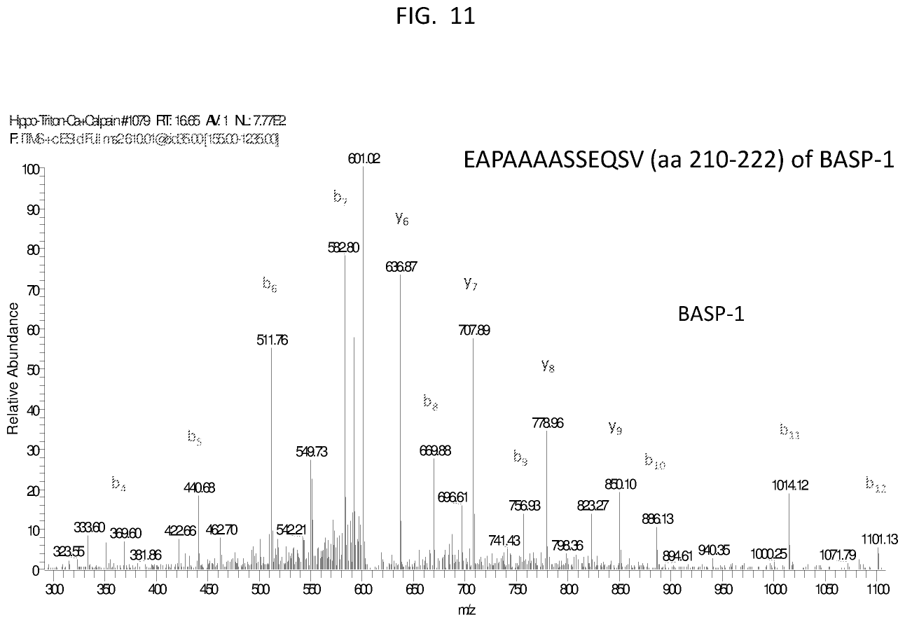

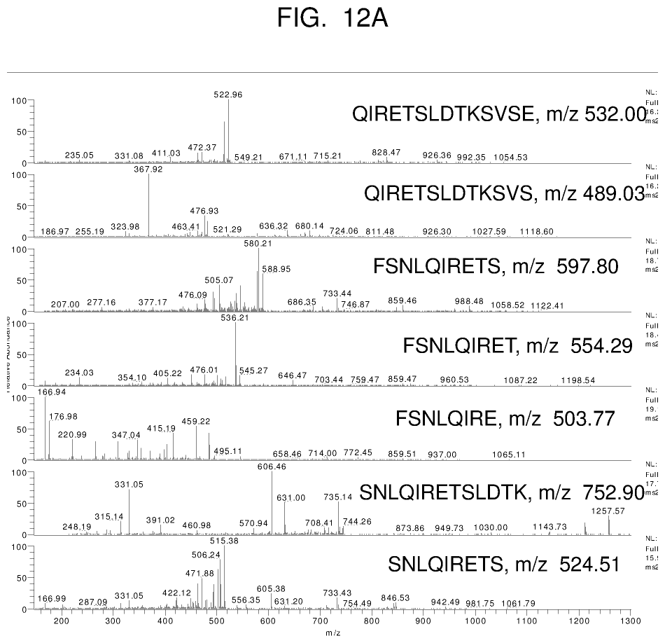

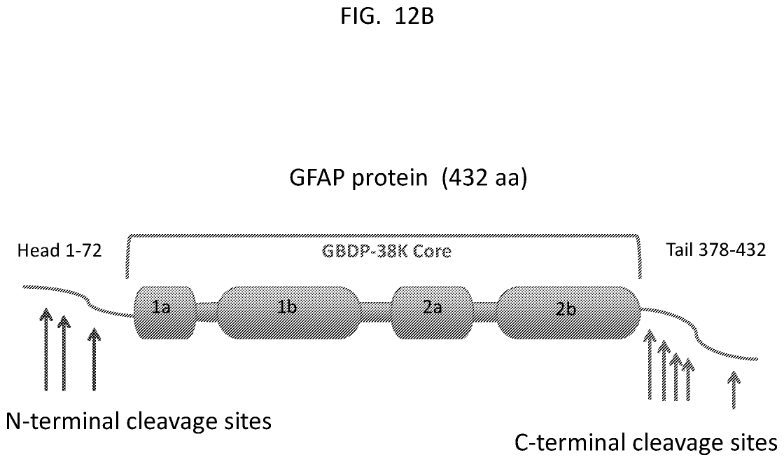

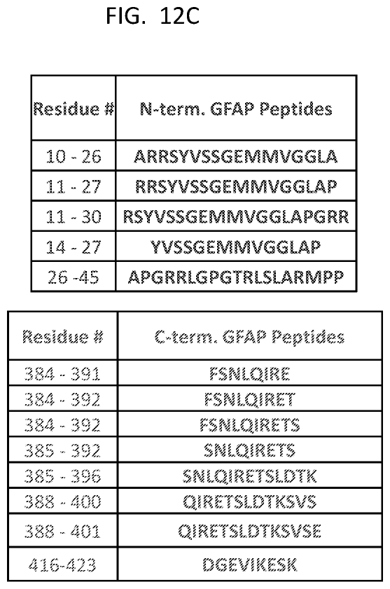

FIG. 5, FIG. 6, FIG. 7 present data showing NRGN breakdown products identified in mouse brain lysates after brain injury. Several different PFs are listed, showing that NRGN breakdown products can indicate an injury to the central nervous system. FIG. 8 and FIG. 9 relates to VIM breakdown products identified in samples taken at days 1, 3, and 7 after injury versus control. FIG. 10 relates to myelin basic protein identified in two brain areas. FIG. 11 presents data identifying breakdown of BASP-1 protein. FIG. 12 shows a schematic of the structure of GFAP, showing multiple cleavage sites (indicated by arrows) when digested by calpain, a cellular calcium dependent protease that is hyperactivated in the brain after TBI, and data concerning identified PFs. Thus, in vitro digestion of central nervous system proteins with calpain mimics injury to the central nervous system or TBI conditions and can serve as an in vitro model of such injury. FIG. 13 presents data showing calpain digestion of Tau-441 protein, releasing PFs, as well as the PBP of 40 kDa and 38 kDa.

FIG. 14 through FIG. 33 present data showing identification of PFs identified in mouse CCI model brain injury lysates and from human CSF from traumatic brain injury subjects.

### D. Methods of Use

The proteins, PBPs, and PFs described here are identified in a sample from a subject such as a human patient who has suffered an injury to the central nervous system or who is suspected of having suffered such an injury. Preferably, a sample is obtained from the subject within 24 hours of the injury or suspected injury. A series of samples also can be taken over a period of days or weeks so that progress can be determined. The sample preferably is CSF or whole blood/serum. Secondary preferred samples are saliva, urine, nasal fluid and tears.

In the case of diagnosing an acute injury or suspected acute injury, a first sample is taken after the injury, preferably as soon as possible and within 24 hours, and further samples can be taken over a time course to obtain information on continued injury or recovery. Testing can be performed to detect a single protein, PBP, or PF, or a combination of one or more proteins, PBPs, or PFs. In some inventive embodiments, at least one protein, PBP, or PF for each of the injury types in Table 1, above, is tested. A high level of one or more of these (approximately twice the level as found in a control sample or uninjured subject or more) indicates an injury, and the identity of the peptide indicates the particular area that has been injured. A peptide level of about 1.5-2.5 times higher than control, or 2.0-2.5 times higher than control (for example about 1.5, 1.75, 2, 2.25, or 2.5 times higher than control), indicates a mild injury; a peptide level of about 2.5-4.0 times higher than control (for example about 2.5, 2.75, 3.0, 3.25, 3.5, 3.75 or 4 times higher than control) indicates a moderate injury; a peptide level of more than about 4.0 times higher than control (for example 4.25, 4.5, 4.75, 5, 5.25, 5.5, 6, 6.5, 7 or more) indicates a severe injury, with amounts higher than 6 times higher than control indicating a very severe injury.

In the case of diagnosing a chronic injury or a suspected chronic injury, a series of samples are taken periodically so that the results can be compared along a time course as well as compared to a control sample from an uninjured subject or an in vitro sample produced for that purpose. Analyte (protein, PBP, or PF) levels that increase over time indicate a chronic or worsening injury; analyte levels that remain about the same over time indicate a stable state or chronic injury; analyte levels that decrease over time indicate that the injury is improving or is not continuing. The levels for determining the severity of the chronic injury are the same as those discussed above for an acute injury.

The precise testing of the samples to be performed to make a diagnosis can be determined by the routine practitioner, depending on the condition of the patient and the suspected type and severity of the injury. For example, if a particular injury to a brain area or subcellular area is suspected after examination of the subject, the sample can be tested for breakdown products derived from the protein identified as correlating with that particular area in this application so that the diagnosis can be confirmed. If the injury is unknown, a large number of tests or the entire panel of tests for all breakdown products can be performed on the sample to make a specific diagnosis.

A diagnosis of a particular injury is made by comparing the results of a subject sample to an uninjured control. If the subject sample has a significantly higher amount of the diagnostic protein, PBP, or PF than the control, a positive diagnosis can be made.

To determine the severity of an injury or prognosis for the subject, the level of a protein, PBP, or PF, or a battery of proteins, PBPs, and PFs can be compared to control samples of varying injury. For example, higher biofluid levels of one or more of the analytes can be correlated to the severity of traumatic injury, to the likelihood of development of post-trauma complications, or to a poor patient prognosis.

### E. Kits

The invention contemplates kits for testing for brain protein breakdown products as described herein, and can include, for example, one or more of the following: suitable containers and equipment for obtaining a subject sample such as CSF or blood; ultrafiltration cell(s) or units with a molecular weight cutoff of about 10 kDa; one or more antibodies or aptamers that specifically recognize a protein, PBP, or PF according to the invention as described herein; and protein, PBPs, and/or PFs according to the invention as described herein to be used as standards in assays. Alternatively, if a mass spectrometry method is to be used for analyte detection, the kit can include analyte standards to be used as internal standards (spike in) or external standards (side-on-side).

A kit according to the invention comprises components for detecting and/or measuring the breakdown products described herein in a sample from a subject. Preferably, the kit contains a primary antibody or aptamer reagent or reagents that each specifically bind to a peptide breakdown product. The antibodies or aptamers can be organized into groups of reagents that recognize the breakdown products of a single protein or a group of proteins that indicate a certain type of central nervous system injury, if desired. Also, the antibodies or aptamers can be organized into panels of reagents that together can detect the breakdown of some or all of the indicator proteins identified here.

The primary antibodies (preferably monoclonal antibodies or fragments thereof) or aptamers specifically recognize and bind to a single peptide or class of peptides. One or more secondary antibodies (optionally labeled) that bind to the primary antibody or aptamer also can be included, as well as a target antigen (the peptide to be detected in the sample). The secondary antibodies can be, for example, antibodies directed toward the constant region of the primary antibody (optionally IgG) (e.g., rabbit anti-human IgG antibody), which may itself be detectably labeled {e.g., with a radioactive, fluorescent, colorimetric or enzyme label), or which may be detected by a labeled tertiary antibody {e.g., goat anti-rabbit antibody).

The antibody- or aptamer-based detection methods can involve a western blot, immunoassays such as enzyme linked immunosorbant assays (ELISA), sandwich assay, or radioimmunoassay (RIA), mass spectrometry, or antibodies or aptamers can be used in combination with mass spectrometry detection methods (e.g., LC-MS/MS). Any detection assay method for proteins and/or peptides known in the art can be used. Suitable containers for performing the assays also can be included in a kit for convenience. Such assays are well known in the art, and any of these known methods can be used with the invention to detect PBP or PF according to the invention. In certain embodiments of the invention, a fast turn around point-of-care diagnostic biofluid test and device can be deployed in various hospital settings. The test will use a biochip or cartridge that contains one or two biomarker target-specific capture and detection antibodies or aptamers. The POC device ha s receptacle for the biochip or cartridge as well as a part that can generate a readout signal. Commonly for these detection methods, the biomarker readout is in the form of light, chemiluminescence or fluorescence signals, chemoelectric signals, radiation signal or absorbance signals. However, mass spectrometry and tandem mass spectrometry methods might also be employed.

A diagnostic test kit generally includes a cartridge or biochip with embedded capature and/or detecting agents (e.g specific antibodies) for one or more protein, PBP. and/or PF biomarker, along with a companion reader or analyzer with a receptacle for the detection cartridge as well as a component capable of producing a biomarker readout. Alternatively a detection kit can be a sandwich ELISA (with capture and detection antibodies for each biomarker) in a singlet or multiplex fashion, as it is commonly described in the field of diagnostics. The detection kit also can be an immunoblotting or western blotting format, as it is commonly described in the field of biochemistry and diagnostics. The common readout from the above mentioned test kits is in the form of light signals (e.g. fluorescence, chemiluminescence), absorbance changes or electrochemical signals. However, mass spectrometry and tandem mass spectrometry methods might also be employed.

Preferably, instructions are packaged with the other components of the kits of the invention, for example, a pamphlet or package label. The instructions explain how to perform testing and methods according to the invention.

In some embodiments, a diagnostic kit comprises (a) detection agents for antibody, aptamer or mass spectrometry detection methods for detection of one or more PFs or other analytes, (b) an analyte protein, protein breakdown product, or PF to serve as internal standard and/or positive control; and (c) a signal generation coupling component. Such signal generation components either are based on detection tool (e.g. antibody) coupled enzyme, which carries out enzymatic reaction to generate a product or direct coupled of a tagging molecule to the detection tool (e.g. antibodies). These enzymatic protein or ragging molecules generally product a light, fluorescence, or chemiluminescence signal, or absorbance changes or electrochemical signals, or the like, to allow detection. However, mass spectrometry and tandem mass spectrometry methods might also be employed.

### 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Unless defined, otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

The following examples are provided as illustrations of the invention and are in no way to be considered limiting.

### Example 1

### Specific Methods

1. Sample Collection and Preparation

Brain samples from CB57BL/6 male mice, 3 to 4 months old, were used. Cortex, corpus callosum and hippocampus regions were isolated from each mouse brain. The brain samples were pulverized to powder using mortar and pestle placed over dry ice to maintain a cold environment. The pulverized brain samples were then lysed using Triton lysis buffer (20 mM Tris-CHl, 5 mM EGTA, 100 mM NaCl, with 1% Triton) by incubating at 4° C. for 90 minutes. After incubation, the samples were centrifuged and a protein assay was performed to estimate the concentration of the mouse brain lysates. Brain lysate equivalent to 120 μg of protein was used.

For some samples, purified protein (GFAP, MBP, NRGN (2-10 ug)), or brain lysate (50-160 ug) were subjected to in vitro incubation with 7 mM calcium chloride CaCl₂) or with calcium and human calpain-1 protease (protease: brain protein ratio of 1:20 to 1:50) and 20 mM (NH₄)₂CO₃, 10 mM dithiothreitol (DTT) and 7 mM CaCl₂) (pH 7.4). This condition mimics the brain injury induced calpain activation in animal and human brain, and serves as an in vitro model of central nervous system injury.

Centrifuged CSF samples (500 uL) were obtained from human subjects with severe TBI (Glasgow coma score of 3-8) and from control, uninjured subjects.

Ultrafiltration was used to separate smaller from larger peptide molecules. The brain lysate and the CSF samples were filtered through 10,000 Da molecular weight cutoff membrane filters (Sartorius Stedim Biotech®, Goettingen, Germany). This filtration technique allows the isolation in the ultrafiltrate of molecules that are smaller than or equal to 10,000 Da, from the retentate.

The ultrafiltrate then was concentrated using a vacuum evaporation method (SpeedVac™; (Thermo Scientific®) to a volume of 5 μL. The concentrated samples were reconstituted with water containing 0.1% formic acid. These samples were ready for liquid chromatography-tandem mass spectrometry. The samples of retentate of ultrafiltration were analyzed using western immunoblotting methods.

### 2. Mass Spectrometry

Tandem mass spectrometry-based proteomic methods first were used to identify PFs derived from the brain injury protein biomarkers using in vitro calcium or calpain digestion of purified protein or TBI-model mouse brain lysate. The samples were analyzed using a system with a Thermo Scientific® LTQ-XL (Thermo Fisher Scientific®, San Jose, Calif., USA) with a Waters® nanoACQUITY UPLC system ((Waters®, Milford, Mass., USA). LC-MS grade water and acetonitrile, both with 0.1% formic acid, were used as mobile phases with a 115 minute gradient at a flow rate of 300 nL/min on a 1.7 μm BEH130 C18 column (100 μm×100 mm). Tandem mass spectra with data-dependent acquisition (top 10 most abundant ions) method was performed using Xcalibur® 2.0.7 (Thermo®). MS/MS data were searched using Proteome Discoverer® 1.3 (Thermo®) against mouse database and human database respectively with no enzyme.

### 3. Western Blotting

Western blot was performed on the higher molecular weight proteins (greater than about 10,100 Da) that were retained on the membrane filter. Western blot was used to confirm proteolysis of proteins in the CCI and TBI samples. SDS-gel electrophoresis and immunoblotting was done using standard published methods (see Yang et al., PLOS ONE 5, e15878, 2010). Blotting membrane was probed with specific target-based antibody (1/500 to 1/2,000 dilution) followed by secondary anti-mouse or anti-rabbit HRP (horse radish peroxidase) conjugate antibody and then detected visually using 5-Bromo-4-chloro-3-indolyl phosphate/Nitro blue tetrazolium (NBT/BCIP) as substrate (colorimetric development). Other immunological assays, such as ELISA (i.e., sandwich assays), RIA, and others known in the art can be used to detect and quantitate the proteins, PBPs and PFs according to the invention, as is convenient to the practitioner. In general, immunological assays such as a sandwich ELISA are preferable for detection of larger peptides and proteins.

### Example 2

### Animal Models

In order to produce an in vivo model of traumatic brain injury in mice, a controlled cortical impact (CCI) device was used according to known methods (see Yang et al., J. Cerebral Blood Flow Metab. 34:1444-1452, 2014). CB57BL/6 mice (male, 3 to 4 months old, Charles River Laboratories®, Raleigh, N.C., USA) were anesthetized with 4% isoflurane in oxygen as a carrier gas for 4 minutes followed by maintenance anesthesia of 2% to 3% isoflurane. After reaching a deep plane of anesthesia, mice were mounted in a stereotactic frame in a prone position, and secured by ear and incisor bars. A midline cranial incision was made and a unilateral (ipsilateral) craniotomy (3 mm diameter) was performed adjacent to the central suture, midway between the bregma and the lambda. The dura mater was kept intact over the cortex. Brain trauma was induced using a PSI TBI-0310 Impactor (Precision Systems and Instrumentation®, LLC, Natick, Mass., USA) by impacting the right cortex (ipsilateral cortex) with 2 mm diameter impactor tip at a velocity of 3.5 m/second, 1.5 mm compression depth, and a 200 millisecond dwell time (compression duration). Sham-injured control animals underwent identical surgical procedures but did not receive an impact injury. Naïve animals underwent no procedure.

### Example 3

### General Methods

See FIG. 2 for a schematic representation of methods used to detect central nervous system biomarker peptides. The figure shows the steps used to identify brain PBPs in samples from a subject. This example shows a method that uses ultrafiltration to separate the low molecular weight PFs from the large proteins or PBPs of greater than about 10,100 Da. Filtrate can be analyzed by the indicated methods to monitor protein degradation derived PFs, while the retentate can be used to monitor the larger PBPs. Any known methods for detection and assay of the proteins, PBPs, and PFs are contemplated for use with the invention, as are convenient to the practitioner.

### Example 4

### Localization of Brain Protein-Derived Breakdown Products

FIG. 3 and FIG. 4 show selected anatomical localization and extracellular, cellular and subcellular locations of the brain protein-derived PBPs and/or PFs as biomarkers for brain injury. The anatomical location of proteolytically vulnerable proteins identified in this application include myelin basic protein (MBP) and Golli-MBP (subcortical white matter), striatin (striatum) and Cortexin-1 (cortex). See FIG. 3.

From the list of mass spectrometry data on PFs obtained using database searches and complimentary immunblotting evidence from protein digestions on samples from a mouse model of TBI and human central nervous system injured subject biofluid samples (based on the peptide XCorr valuses (e.g., XCorr >3.0), brain cell type specific markers identified in this application include the proteins in Table 2, above. See also FIG. 4.

### Example 5

### TBI-Induced Peptide Fragments in Mouse Cortex and Hippocampus

Mice subjected to traumatic brain injury as described in Example 2 were sacrificed. Cortex and hippocampus tissue sample lysates were subjected to ultrafiltration and the ultrafiltrates tested by nLC-MSMS to identify TBI-induced PFs. The PFs were identified by comparison with immunoblotting data on proteins/PBPs. Results are shown in Table 3, below. The data showed that the in vitro incubation model and the mouse model of TBI both resulted in production of similar brain PBPs/PFs than those found in the CSF samples of human TBI subjects. PBPs or PFs identified by all three methods therefore can have utility in diagnosing or monitoring human brain damage.

TABLE 3
Identification of TBI-induced Peptide Fragments from mouse CCI cortex and
hippocampal samples (ultrafiltrate by nLC-MSMS).

| Mouse | | | | |
|---|---|---|---|---|
| Accession | | Mouse | Mol. Wt. | |
| Number | Protein Name | Gene | (kDa) | Origin |
| Q8R5A3 | Amyloid beta A4 precursor protein-binding | Apbb1ip | 74.3 | Neuron-axonal |
| | family B member 1-interacting protein | | | |
| H7BX08 | Calmodulin-regulated spectrin-associated | Camsap2 | 166 | Neuron-axonal |
| | protein 2 | | | |
| E9Q5B0 | Calmodulin-regulated spectrin-associated | Camsap3 | 135.2 | Neuron-axonal |
| | protein 3 | | | |
| Q8VHY0 | Chondroitin sulfate proteoglycan 4 | Cspg4 | 252.2 | Extracell. Matrix |
| D6R934 | Complement Protein C1q | C1q | 26.7 | Microglia/ |
| | | | | macrophage |
| P09871 | Complement Protein C1s | C1s | 76.6 | Microglia/ |
| | | | | macrophage |
| P01024 | Complement Protein C3 | C3 | 180.0 | Microglia/ |
| | | | | macrophage |
| P01031 | Complement Protein C5 | C5 | 188.3 | Microglia/ |
| | | | | macrophage |
| E9PDY4; | Complement C3b/C4b receptor | CR1 | 223.6 | Microglia/ |
| P17927 | receptor CR1 (CD35) | | | macrophage |
| P20023 | Complement receptor | CR2 | 110.0 | Microglia/ |
| | receptor 2, CR2 (CD21) | | | macrophage |
| Q91XM9 | Disks large homolog 2, PSD98 | Dlg2 | 103 | Post-synaptic |
| | | | | term. |
| P03995-2 | Glial fibrillary acidic protein | Gfap | 49.8 | Astrocyte |
| P48318 | Glutamate decarboxylase 1 | Gad1 | 66.6 | Neuron-cell |
| | | | | body |
| G3X9H5 | Huntingtin | Htt | 344.6 | Neuron-cell |
| | | | | body |
| Q9Z0E0-2 | Neurochondrin | Ncdn | 77.4 | Extracell.. |
| | | | | matrix |
| Q61830 | Macrophage mannose receptor 1 (MRC1, | Mrc1 | 1456 | Microglia/ |
| | CD206) | | | macrophage |
| A2ARP8 | Microtubule-associated protein 1A | Map1a | 325.7 | Neuron-dendritic |
| P14873 | Microtubule-associated protein 1B | Map1b | 270.1 | Neuron-dendritic |
| P20357 | Microtubule-associated protein 2 | Map2 | 199 | Neuron-dendritic |
| Q7TSJ2 | Microtubule-associated protein 6 | Map6 | 96.4 | Neuron-dendritic |
| P04370 | Myelin A1 protein (Golli-MBP) | Mbp(A1) | 27.2 | Oligodendrocyte |
| P04370-4 | myelin basic protein isoform 4 | MBP (4) | 21.5 | Oligodendrocyte |
| Q3UR85 | Myelin regulatory factor | MYRF | 123.3 | Oligodendrocyte |
| P60761 | Neurogranin | NRGN | 8.2 | Post-synaptic |
| | | | | term. |
| E0CY11 | Neurexin-1 | Nrxn1 | 164.1 | Pre-synaptic |
| | | | | term. |
| Q6P9K9 | Neurexin-3 | Nrxn3 | 173.3 | Pre-synaptic |
| | | | | term. |
| P55066 | Neurocan core protein | Ncan | 137.1 | Extracell. Matrix |
| E9PW06 | Neurofascin | Nfasc | 132.1 | Neuron-axonal |
| P19246 | Neurofilament heavy polypeptide | Nefh | 116.9 | Neuron-axonal |
| P08551 | Neurofilament light polypeptide | Nef1 | 61.5 | Neuron-axonal |
| Q03517 | Secretogranin-2 | Scg | 70.6 | Pre-synaptic |
| | | | | term. |
| A3KGU5 | Spectrin alpha chain, non-erythrocytic 1 | Sptan1 | 282.7 | Neuron-axonal |
| Q62261 | Spectrin beta chain, non-erythrocytic 1 | Sptbn1 | 274.1 | Neuron-axonal |
| | (isoform 2) | | | |
| Q64332-2 | Synapsin-2 (isoform IIb) | Syn | 63.3 | Pre-synaptic |
| | | | | term. |
| P46097 | Synaptotagmin-2 | Syt2 | 47.2 | Pre-synaptic |
| | | | | term. |
| Q9D6F9 | Tubulin beta-4A chain | Tubb4a | 49.6 | Neuron-dendritic |
| P68372 | Tubulin beta-4B chain | Tubb4b | 49.8 | Neuron-dendritic |
| P20152-1 | Vimentin | Vim | 53.7 | astrocyte |

### Example 6

### Identification of Neurogranin Peptide in Mouse Brain Lysate Ultrafiltrate

FIG. 5 shows exemplary LC-MS/MS evidence for NRGN PF PGANAAAAKIQASFRGHMARKKIKSGECGRKGPGG (aa 24-63; SEQ ID NO:185) in the ultrafiltrate portion of brain lysate (molecular weight cutoff 10,000 Da) after TBI in mice. FIG. 6 shows an MS/MS spectrum of the NRGN PF DDDILDIPLDDPGANAAAAKIQASFR (SEQ ID NO:186) released from ipsilateral cortex CCI (day 7) injury in mice. See Tables 4 and 5 for the specific data for FIG. 5 and FIG. 6, respectively. Italic and Underlined peptide ions are the b and y peptide ions identified by MS/MS spectrum, respectively.

TABLE 4
MS/MS Data for FIG. 5.

| | | | | SEQ | | | | |
|---|---|---|---|---|---|---|---|---|
| | | | | ID | | | | |
| | | | | NO: | | | | |
| #1 | b⁺ | b²⁺ | b³⁺ | 187 | y⁺ | y²⁺ | y³⁺ | #2 |
| 1 | 98.06 | 49.53 | 33.36 | P | | | | 37 |
| 2 | 155.08 | 78.04 | 52.37 | G | 3637.90 | 1819.45 | 1213.31 | 36 |
| 3 | 226.12 | 113.56 | 76.04 | A | 3580.88 | 1790.94 | 1194.30 | 35 |
| 4 | 340.16 | 170.58 | 114.06 | N | 3509.84 | 1755.43 | 1170.62 | 34 |
| 5 | 411.20 | 206.10 | 137.74 | A | 3395.80 | 1698.40 | 1132.61 | 33 |
| 6 | 482.24 | 241.62 | 161.42 | A | 3324.76 | 1662.89 | 1108.93 | 32 |
| 7 | 553.27 | 277.14 | 185.10 | A | 3253.73 | 1627.37 | 1085.25 | 31 |
| 8 | 624.31 | 312.66 | 208.77 | A | 3182.69 | 1591.85 | 1061.57 | 30 |
| 9 | 752.40 | 376.71 | 251.47 | K | 3111.65 | 1556.33 | 1037.89 | 29 |
| 10 | 865.49 | 433.25 | 289.17 | I | 2983.56 | 1492.28 | 995.19 | 28 |
| 11 | 993.55 | 497.28 | 331.85 | Q | 2870.47 | 1435.74 | 957.50 | 27 |
| 12 | 1064.58 | 532.80 | 355.53 | A | 2742.42 | 1371.71 | 914.81 | 26 |
| 13 | 1151.62 | 576.31 | 384.54 | S | 2671.38 | 1336.19 | 891.13 | 25 |
| 14 | 1298.69 | 649.85 | 433.57 | F | 2584.35 | 1292.68 | 862.12 | 24 |
| 15 | 1454.79 | 727.90 | 485.60 | R | 2437.28 | 1219.14 | 813.10 | 23 |
| 16 | 1511.81 | 756.41 | 504.61 | G | 2281.18 | 1141.09 | 761.06 | 22 |
| 17 | 1648.87 | 824.94 | 550.29 | H | 2224.15 | 1112.58 | 742.06 | 21 |
| 18 | 1795.90 | 898.45 | 599.31 | M | 2087.10 | 1044.05 | 696.37 | 20 |
| | | | | (ox) | | | | |
| 19 | 1866.94 | 933.97 | 622.98 | A | 1940.06 | 970.53 | 647.36 | 19 |
| 20 | 2023.04 | 1012.02 | 675.02 | R | 1869.02 | 935.02 | 623.68 | 18 |
| 21 | 2151.14 | 1076.07 | 717.72 | K | 1712.92 | 856.96 | 571.65 | 17 |
| 22 | 2279.23 | 1140.12 | 760.41 | K | 1584.83 | 792.92 | 528.95 | 16 |
| 23 | 2392.31 | 1196.66 | 798.11 | I | 1456.73 | 728.87 | 486.25 | 15 |
| 24 | 2520.41 | 1260.71 | 840.81 | K | 1343.65 | 672.33 | 448.55 | 14 |
| 25 | 2607.44 | 1304.22 | 869.82 | S | 1215.55 | 608.28 | 405.86 | 13 |
| 26 | 2664.46 | 1332.74 | 888.83 | G | 1128.52 | 564.76 | 376.85 | 12 |
| 27 | 2793.51 | 1397.26 | 931.84 | E | 1071.50 | 536.25 | 357.84 | 11 |
| 28 | 2953.54 | 1477.27 | 985.18 | C | 942.46 | 471.73 | 314.82 | 10 |
| | | | | (carb) | | | | |
| 29 | 3010.56 | 1505.78 | 1004.19 | G | 782.43 | 391.72 | 261.48 | 9 |
| 30 | 3166.66 | 1583.83 | 1056.22 | R | 725.41 | 363.21 | 242.47 | 8 |
| 31 | 3294.75 | 1647.88 | 1098.92 | K | 569.30 | 285.16 | 190.44 | 7 |
| 32 | 3351.77 | 1676.39 | 1117.93 | G | 441.21 | 221.11 | 147.74 | 6 |
| 33 | 3448.83 | 1724.92 | 1150.28 | P | 384.19 | 192.60 | 128.73 | 5 |
| 34 | 3505.85 | 1753.43 | 1169.29 | G | 287.14 | 144.07 | 96.38 | 4 |
| 35 | 3602.90 | 1801.95 | 1201.64 | P | 230.11 | 115.56 | 77.38 | 3 |
| 36 | 3659.92 | 1830.47 | 1220.65 | G | 133.06 | 67.03 | 45.03 | 2 |
| 37 | | | | G | 76.04 | 38.52 | 26.02 | 1 |

TABLE 5
MS/MS Data for FIG. 6.

| | | | | SEQ | | | | |
|---|---|---|---|---|---|---|---|---|
| | | | | ID | | | | |
| | | | | NO: | | | | |
| #1 | b⁺ | b²⁺ | b³⁺ | 188 | y⁺ | y²⁺ | y³⁺ | #2 |
| 1 | 116.03 | 58.52 | 39.35 | D | | | | 26 |
| 2 | 231.06 | 116.03 | 77.69 | D | 2597.32 | 1299.16 | 866.45 | 25 |
| 3 | 346.09 | 173.55 | 116.03 | D | 2482.29 | 1241.65 | 828.10 | 24 |
| 4 | 459.17 | 230.09 | 153.73 | I | 2367.27 | 1184.14 | 789.76 | 23 |
| 5 | 572.26 | 286.63 | 191.42 | L | 2254.18 | 1127.59 | 752.07 | 22 |
| 6 | 687.28 | 344.15 | 229.77 | D | 2141.10 | 1071.05 | 714.37 | 21 |
| 7 | 800.37 | 400.69 | 267.46 | I | 2026.07 | 1013.54 | 676.03 | 20 |
| 8 | 897.42 | 449.21 | 299.81 | P | 1912.99 | 957.00 | 638.33 | 19 |
| 9 | 1010.50 | 505.76 | 337.51 | L | 1815.93 | 908.47 | 605.98 | 18 |
| 10 | 1125.53 | 563.27 | 375.85 | D | 1702.85 | 851.93 | 568.29 | 17 |
| 11 | 1240.56 | 620.78 | 414.19 | D | 1587.82 | 794.42 | 529.95 | 16 |
| 12 | 1337.61 | 669.31 | 446.54 | P | 1472.80 | 736.90 | 491.60 | 15 |
| 13 | 1394.63 | 697.82 | 465.55 | G | 1375.74 | 688.38 | 459.25 | 14 |
| 14 | 1465.67 | 733.34 | 489.23 | A | 1318.72 | 659.86 | 440.25 | 13 |
| 15 | 1579.71 | 790.36 | 527.24 | N | 1247.69 | 624.35 | 416.57 | 12 |
| 16 | 1650.75 | 825.88 | 550.92 | A | 1133.64 | 567.32 | 378.55 | 11 |
| 17 | 1721.79 | 861.40 | 574.60 | A | 1062.61 | 531.81 | 354.87 | 10 |
| 18 | 1792.82 | 896.92 | 598.28 | A | 991.57 | 496.29 | 331.19 | 9 |
| 19 | 1863.86 | 932.43 | 621.96 | A | 920.53 | 460.77 | 307.52 | 8 |
| 20 | 1991.96 | 996.48 | 664.66 | K | 849.49 | 425.25 | 283.84 | 7 |
| 21 | 2105.04 | 1053.02 | 702.35 | I | 721.40 | 361.20 | 241.14 | 6 |
| 22 | 2233.10 | 1117.05 | 745.04 | Q | 608.32 | 304.66 | 203.44 | 5 |
| 23 | 2304.14 | 1152.57 | 768.72 | A | 480.26 | 240.63 | 160.76 | 4 |
| 24 | 2391.17 | 1196.09 | 797.73 | S | 409.22 | 205.11 | 137.08 | 3 |
| 25 | 2538.24 | 1269.62 | 846.75 | F | 322.19 | 161.60 | 108.07 | 2 |
| 26 | | | | R | 175.12 | 88.06 | 59.04 | 1 |

NGRN (NP_071312) PFs identified in TBI mice brain lysate ultrafiltrate samples are given in FIG. 7. In the mouse NRGN protein (MDCCTESACSKPDDDILDIPLDDPGANAAAAKIQASFRGHMARKKIKSGECGRKGPGP GGPGGAGGARGGAGGGPSGD; SEQ ID NO:189) the underlined portion represents the area which contained the detected PFs. Duplicate peptides found are not shown. None of the PFs shown were found in native (control) mouse cortex samples. The residue number is shown on the X-axis.

FIGS. 7A and 7B show the ipsilateral cortex profile of the NRGN fragmentation pattern at different time points (day 1, day 7) after CCI and repetitive closed head injury (rCHI) in mice. FIGS. 7C and 7D show the same data for hippocampus. FIG. 7A and FIG. 7C are western blots of NRGN and the PBP of NRGN, visualized using an internal epitope antibody (EMD AB5620), with internal loading control β-actin (43 kDa). Intact NRGN appears as 14 kDa band, while a major PF appears as a 7 kDa band. FIG. 7B and FIG. 7D show the densitometric quantitation of the intact protein and PBP/PF of NRGN. Error bars represent the standard error of the mean (N=3). * indicates a statistical significance compared to naive (p-value <0.05) (2 tailed unpaired T-test). This example shows that biofluid-based monitoring of NRGN fragments can be used to monitor presynaptic terminal damage.

### Example 7

### Identification of Vimentin Peptide in Mouse Brain Lysate Ultrafiltrate

FIG. 8A and FIG. 8B show data characterizing exemplary VIM proteolytic breakdown products (peptides) in the ultrafiltrate portion of mouse cortical lysate after TBI. The MS/MS spectrum of the peptide GSGTSSRPSSNRSYVTTSTRTYSLGSALRPSTSR (VIM aa 17-50; SEQ ID NO:190), charge+2, monoisotopic m/z 1902.83 Da, displays the fragment ions for this peptide (FIG. 8A). Identified are b+ and y+ type ions for the VIM peptide shown in italics and underline. FIG. 8B shows an MS/MS spectrum of the peptide NLESLPLVDTHSKRTLLIKTVETRDGQVINE (VIM aa 426-456; SEQ ID NO:191), charge +2, monoisotopic m/z 1902.83 Da, displaying the fragment ions for this peptide. See Tables 6 and 7 for the data accompanying FIGS. 8A and 8B, respectively. Italic and Underlined peptide ions are the b and y peptide ions identified by MS/MS spectra, respectively.

TABLE 6
MS/MS Data for FIG. 8A.

| | | | SEQ ID | | | |
|---|---|---|---|---|---|---|
| #1 | b⁺ | b²⁺ | NO:192 | y⁺ | y²⁺ | #2 |
| 1 | 58.03 | 29.52 | G | | | 34 |
| 2 | 225.03 | 113.02 | S (p) | 3747.65 | 1874.33 | 33 |
| 3 | 282.05 | 141.53 | G | 3580.65 | 1790.83 | 32 |
| 4 | 383.10 | 192.05 | T | 3523.63 | 1762.32 | 31 |
| 5 | 550.09 | 275.55 | S (p) | 3422.58 | 1711.80 | 30 |
| 6 | 717.09 | 359.05 | S (p) | 3255.59 | 1628.30 | 29 |
| 7 | 873.19 | 437.10 | R | 3088.59 | 1544.80 | 28 |
| 8 | 970.25 | 485.63 | P | 2932.49 | 1466.75 | 27 |
| 9 | 1057.28 | 529.14 | S | 2835.43 | 1418.22 | 26 |
| 10 | 1144.31 | 572.66 | S | 2748.40 | 1374.70 | 25 |
| 11 | 1258.35 | 629.68 | N | 2661.37 | 1331.19 | 24 |
| 12 | 1414.45 | 707.73 | R | 2547.33 | 1274.17 | 23 |
| 13 | 1501.49 | 751.25 | S | 2391.23 | 1196.12 | 22 |
| 14 | 1664.55 | 832.78 | Y | 2304.19 | 1152.60 | 21 |
| 15 | 1763.62 | 882.31 | V | 2141.13 | 1071.07 | 20 |
| 16 | 1864.67 | 932.84 | T | 2042.06 | 1021.53 | 19 |
| 17 | 1965.71 | 983.36 | T | 1941.01 | 971.01 | 18 |
| 18 | 2052.75 | 1026.88 | S | 1839.97 | 920.49 | 17 |
| 19 | 2153.79 | 1077.40 | T | 1752.94 | 876.97 | 16 |
| 20 | 2309.89 | 1155.45 | R | 1651.89 | 826.45 | 15 |
| 21 | 2410.94 | 1205.97 | T | 1495.79 | 748.40 | 14 |
| 22 | 2574.01 | 1287.51 | Y | 1394.74 | 697.87 | 13 |
| 23 | 2661.04 | 1331.02 | S | 1231.68 | 616.34 | 12 |
| 24 | 2774.12 | 1387.56 | L | 1144.64 | 572.83 | 11 |
| 25 | 2831.14 | 1416.08 | G | 1031.56 | 516.28 | 10 |
| 26 | 2918.18 | 1459.59 | S | 974.54 | 487.77 | 9 |
| 27 | 2989.21 | 1495.11 | A | 887.51 | 444.26 | 8 |
| 28 | 3102.30 | 1551.65 | L | 816.47 | 408.74 | 7 |
| 29 | 3258.40 | 1629.70 | R | 703.38 | 352.20 | 6 |
| 30 | 3355.45 | 1678.23 | P | 547.28 | 274.15 | 5 |
| 31 | 3442.48 | 1721.74 | S | 450.23 | 225.62 | 4 |
| 32 | 3543.53 | 1772.27 | T | 363.20 | 182.10 | 3 |
| 33 | 3630.56 | 1815.78 | S | 262.15 | 131.58 | 2 |
| 34 | | | R | 175.12 | 88.06 | 1 |

TABLE 7
MS/MS Data for FIG. 8B.

| | | | | SEQ | | | | |
|---|---|---|---|---|---|---|---|---|
| | | | | ID | | | | |
| | | | | NO: | | | | |
| #1 | b⁺ | b²⁺ | b³⁺ | 193 | y⁺ | y²⁺ | y³⁺ | #2 |
| 1 | 115.05 | 58.03 | 39.02 | N | | | | 31 |
| 2 | 228.13 | 114.57 | 76.72 | L | 3564.8 | 1782.9 | 1188.9 | 30 |
| 3 | 357.18 | 179.09 | 119.73 | E | 3451.7 | 1726.4 | 1151.2 | 29 |
| 4 | 524.18 | 262.59 | 175.40 | S | 3322.7 | 1661.8 | 1108.2 | 28 |
| | | | | (p) | | | | |
| 5 | 637.26 | 319.13 | 213.09 | L | 3155.7 | 1578.3 | 1052.6 | 27 |
| 6 | 734.31 | 367.66 | 245.44 | P | 3042.6 | 1521.8 | 1014.9 | 26 |
| 7 | 847.40 | 424.20 | 283.14 | L | 2945.5 | 1473.3 | 982.5 | 25 |
| 8 | 946.46 | 473.74 | 316.16 | V | 2832.5 | 1416.7 | 944.8 | 24 |
| 9 | 1061.49 | 531.25 | 354.50 | D | 2733.4 | 1367.2 | 911.8 | 23 |
| 10 | 1162.54 | 581.77 | 388.18 | T | 2618.4 | 1309.7 | 873.5 | 22 |
| 11 | 1299.60 | 650.30 | 433.87 | H | 2517.3 | 1259.2 | 839.8 | 21 |
| 12 | 1466.60 | 733.80 | 489.54 | S | 2380.3 | 1190.6 | 794.1 | 20 |
| | | | | (p) | | | | |
| 13 | 1594.69 | 797.85 | 532.24 | K | 2213.3 | 1107.1 | 738.4 | 19 |
| 14 | 1750.79 | 875.90 | 584.27 | R | 2085.2 | 1043.1 | 695.7 | 18 |
| 15 | 1851.84 | 926.42 | 617.95 | T | 1929.1 | 965.0 | 643.7 | 17 |
| 16 | 1964.92 | 982.97 | 655.65 | L | 1828.0 | 914.5 | 610.0 | 16 |
| 17 | 2078.01 | 1039.51 | 693.34 | L | 1714.9 | 858.0 | 572.3 | 15 |
| 18 | 2191.09 | 1096.05 | 731.04 | I | 1601.8 | 801.4 | 534.6 | 14 |
| 19 | 2319.19 | 1160.10 | 773.73 | K | 1488.8 | 744.9 | 496.9 | 13 |
| 20 | 2420.24 | 1210.62 | 807.42 | T | 1360.7 | 680.8 | 454.2 | 12 |
| 21 | 2519.30 | 1260.16 | 840.44 | V | 1259.6 | 630.3 | 420.5 | 11 |
| 22 | 2648.35 | 1324.68 | 883.45 | E | 1160.6 | 580.8 | 387.5 | 10 |
| 23 | 2749.39 | 1375.20 | 917.14 | T | 1031.5 | 516.3 | 344.5 | 9 |
| 24 | 2905.49 | 1453.25 | 969.17 | R | 930.5 | 465.7 | 310.8 | 8 |
| 25 | 3020.52 | 1510.76 | 1007.51 | D | 774.4 | 387.7 | 258.8 | 7 |
| 26 | 3077.54 | 1539.28 | 1026.52 | G | 659.3 | 330.2 | 220.5 | 6 |
| 27 | 3205.60 | 1603.30 | 1069.21 | Q | 602.3 | 301.7 | 201.4 | 5 |
| 28 | 3304.67 | 1652.84 | 1102.23 | V | 474.3 | 237.6 | 158.8 | 4 |
| 29 | 3417.75 | 1709.38 | 1139.92 | I | 375.2 | 188.1 | 125.7 | 3 |
| 30 | 3531.80 | 1766.40 | 1177.94 | N | 262.1 | 131.6 | 88.0 | 2 |
| 31 | | | | E | 148.1 | 74.5 | 50.0 | 1 |

FIG. 9A and FIG. 9B show the profiles of ipsilateral cortex of the VIM fragmentation pattern at different time points (day 1, day 3, day 7) after CCI in mice. FIG. 9A is a western blot showing the PBP of VIM visualized using an internal epitope antibody (Abcam ab92547) with internal loading control β-actin (43 kDa). Intact VIM appears as a 50 kDa band, while the major higher molecular weight PBPs appear as 48 and 38 kDa bands. FIG. 9B is a densitometric quantitation of intact VIM and PBPs of the VIM protein. Error bars represent the standard error of the mean (N=3). * shows statistical significance compared to naive (p-value <0.05) (2 tailed unpaired T-test). FIG. 9C and FIG. 9D present the same date for VIM fragmentation in mouse hippocampus. These data show shows that biofluid-based monitoring of VIM PBPs or PFs can be used to monitor astroglia injury mediated by calpain activation.

### Example 8

### Characterization of Myelin Basic Protein Protein Breakdown Products and Peptide Fragments in Mouse Brain after TBI

FIG. 10 presents data characterizing myelin basic protein (isoform 4 or isoform 5) peptide release and concomitant PBP formation in mouse hippocampal and corpus callosum lysate after TBI. FIG. 10A shows MS/MS spectrum of the mouse MBP peptide KNIVTPRTPPP (residues 115-125; SEQ ID NO:195) based on mouse MBP isoform 4 (NP_001020422), 195 aa), released from ipsilateral cortex CCI on day 1 after injury in mice. The MBP peptide appears as a charge of +2, monoisotopic m/z 528.99. The spectrum shows the fragment ions with Identified b+ and y+ type ions in italics and underline, respectively, in Table 8, below.

TABLE 8
MS/MS Data for FIG. 10A.

| | | | SEQ ID | | | |
|---|---|---|---|---|---|---|
| | b⁺ | b²⁺ | NO: 196 | y⁺ | y²⁺ | |
| 1 | 129.18 | 65.09 | K | | | 11 |
| 2 | 243.29 | 122.15 | N | 1092.28 | 546.65 | 10 |
| 3 | 356.45 | 178.73 | I | 978.18 | 489.59 | 9 |
| 4 | 455.58 | 228.29 | V | 865.02 | 433.01 | 8 |
| 5 | 556.68 | 278.85 | T | 765.89 | 383.45 | 7 |
| 6 | 653.80 | 327.40 | P | 664.78 | 332.90 | 6 |
| 7 | 809.99 | 405.50 | R | 567.67 | 284.34 | 5 |
| 8 | 911.09 | 456.05 | T | 411.48 | 206.24 | 4 |
| 9 | 1008.21 | 504.61 | P | 310.37 | 155.69 | 3 |
| 10 | 1105.33 | 553.17 | P | 213.26 | 107.13 | 2 |
| 11 | | | P | 116.14 | 58.57 | 1 |

FIG. 10B and FIG. 10C (corpus callosum) and FIG. 10D and FIG. 10E (hippocampus) present the profile of the myelin basic protein PBPs at different time points (day 1, day 3, day 7) after CCI in mice in the two brain areas as indicated. FIG. 10B and FIG. 10D are western blots showing the myelin basic protein breakdown product (10 kDa or more), visualized with an epitope-specific antibody recognizing the peptide KNIVTPRTPPP (SEQ ID NO:225) and using internal loading of the control β-actin. FIG. 10C and FIG. 10E show the densitometric quantitation of the 10 kDa myelin basic protein breakdown product. Error bars represent the standard error of the mean (N=3)). * shows statistical significance over naive (p-value <0.05) (2 tailed unpaired T-test). Since MBP is derived from oligodendrocytes that form the myelin sheath around axons, formation and release of MBP PBP or PF indicates oligodendrocyte/myelin and white matter damage Thus, this example shows that biofluid-based monitoring of MBP PBP or PFs can be used to monitor oligodendrocyte/myelin damage/white matter injury.

### Example 9

### Characterization of Brain Acidic Soluble Protein 1 Peptides

FIG. 11 shows an MS/MS spectrum displaying the fragment ions for the brain acidic soluble protein 1 (BASP-1) PF: EAPAAAASSEQSV (SEQ ID NO:226) released from a hippocampus lysate digestion with calpain-1 in vitro. Identified b- and y-type ions for the BASP1 peptide are shown. The identified b- and y-type ions for the BASP1 peptide are shown in Table 9, below. This example shows that biofluid-based monitoring of the BASP1 PBPs or PFs can be used to monitor neuronal cell body injury.

TABLE 9
MS/MS Data for FIG. 11.

| | | | SEQ ID | | | |
|---|---|---|---|---|---|---|
| | b⁺ | b²⁺ | NO:227 | y⁺ | y²⁺ | |
| 1 | 130.12299 | 65.56519 | E | | | 13 |
| 2 | 201.201669 | 101.10454 | A | 1089.14629 | 545.07684 | 12 |
| 3 | 298.31849 | 149.66294 | P | 1018.06759 | 509.53749 | 11 |
| 4 | 369.39719 | 185.20229 | A | 920.95079 | 460.97909 | 10 |
| 5 | 440.47589 | 220.74164 | A | 849.87209 | 425.43974 | 9 |
| 6 | 511.55459 | 256.28099 | A | 778.79339 | 389.90039 | 8 |
| 7 | 582.63329 | 291.82034 | A | 707.71469 | 354.36104 | 7 |
| 8 | 669.71129 | 335.35934 | S | 636.63599 | 318.82169 | 6 |
| 9 | 756.78929 | 378.89834 | S | 549.55799 | 275.28269 | 5 |
| 10 | 885.90489 | 443.45614 | E | 462.47999 | 231.74369 | 4 |
| 11 | 1014.03580 | 507.52160 | Q | 333.36439 | 167.18589 | 3 |
| 12 | 1101.11380 | 551.06060 | S | 205.23348 | 103.12044 | 2 |
| 13 | | | V | 118.15548 | 59.58144 | 1 |

### Example 10

### Human Glial Fibrillary Protein N- and C-Peptidome—In Vitro Calpain Digestion

The peptides identified in this Example show the distinct PFs released into the fluid biological sample ultrafiltrate of in vitro calpain proteolyzed human GFAP protein. This method mimics the human TBI conditions where calpain is known to be hyperactivated and to attack cellular proteins in the brain.

FIG. 12A shows low molecular weight PFs produced from digestion of human GFAP calpain (a cellular protease that is hyperactivated after traumatic brain injury), identified from their MS/MS spectra.

FIG. 12B is a schematic diagram showing the structure of GFAP, including the head and tail sections and the GBDP-38 kDa core section. This linear model of GFAP protein shows the location of N-terminal region (aa 10-45) and C-terminal region (aa 384-423) released PFs as well as the 38 kDa core. FIG. 12C shows the sequences of GFAP peptides from the N-terminus and C-terminus of GFAP.

Table 11 shows the GFAP PFs identified in ultrafiltrate samples from a calpain-digested sample of purified human GFAP protein. The calpain proteolysis mimics CNS traumatic injury-induced calpain activation. A number of GFAP PFs were identified, as shown in Table 11, below.

The sequence of human GFAP (Accession No. P14136; 432 amino acids; GI:251802) is as below (regions with GFAP PFs identified are shown in bold).

SEQ ID NO: 236
MERRRITSAARRSYVSSGEMMVGGLAPGRRLGPGTRLSLARMPPPLPTRV
DFSLAGALNAGFKETRASERAEMMELNDRFASYIEKVRFLEQQNKALAAE
LNQLRAKEPTKLADVYQAELRELRLRLDQLTANSARLEVERDNLAQDLAT
VRQKLQDETNLRLEAENNLAAYRQEADEATLARLDLERKIESLEEEIRFL
RKIHEEEVRELQEQLARQQVHVELDVAKPDLTAALKEIRTQYEAMASSNM
HEAEEWYRSKFADLTDAAARNAELLRQAKHEANDYRRQLQSLTCDLESLR
GTNESLERQMREQEERHVREAASYQEALARLEEEGQSLKDEMARHLQEYQ
DLLNVKLALDIEIATYRKLLEGEENRITIPVQTFSNLQIRETSLDTKSVS
EGHLKRNIVVKTVEMRDGEVIKESKQEHKDVM

TABLE 11
Peptide Fragments Released from Human GFAP (P14136) upon in Vitro Calpain
Proteolysis.

| | | # | | | | MH+ | m/z | SEQ ID |
|---|---|---|---|---|---|---|---|---|
| Sequence | aa # | PSMs | ΔCn | XCorr | Charge | [Da] | [Da] | NO: |
| AARRSYVSSGEMMV | 9-28 | 1 | 0.0000 | 2.05 | 2 | 1997.33 | 998.66 | 237 |
| GGLAPG | | | | | | | | |
| ARRSYVSSGEMMVG | 10-29 | 1 | 0.0000 | 2.23 | 2 | 2079.79 | 1039.89 | 238 |
| GLAPGR | | | | | | | | |
| RRSYVSSGEMmVGG | 11-27 | 1 | 0.0122 | 1.81 | 3 | 1812.07 | 604.02 | 239 |
| LAP | | | | | | | | |
| RSYVSSGEmmVGGL | 11-30 | 1 | 0.0000 | 1.41 | 2 | 2041.77 | 1020.88 | 240 |
| APGRR | | | | | | | | |
| YVSSGEmMVGGLAP | 14-27 | 2 | 0.0000 | 1.80 | 3 | 1414.29 | 471.43 | 241 |
| FSNLQIRET | 384-391 | 2 | 0.0000 | 2.24 | 2 | 1108.15 | 554.08 | 242 |
| FSNLQIRETS | 384-392 | 1 | 0.0000 | 2.35 | 2 | 1194.65 | 597.32 | 243 |
| SNLQIRETSLDTKS | 385-396 | 2 | 0.0000 | 2.20 | 3 | 1593.59 | 531.20 | 244 |
| SNLQIRETSLDTK | 385-396 | 1 | 0.0000 | 1.78 | 3 | 1506.11 | 502.04 | 245 |
| SNLQIRETSLDTKSVS | 385-398 | 1 | 0.0000 | 2.92 | 3 | 1776.46 | 592.15 | 246 |
| QIRETSLDTKSVS | 388-400 | 2 | 0.0000 | 2.55 | 3 | 1464.58 | 488.19 | 247 |
| DGEVIKESK | 416-423 | 9 | 0.0000 | 2.36 | 2 | 1005.51 | 502.76 | 248 |

Since GFAP is a major astrogial protein that is also involved in post-injury gliosis (glia cell hypertrophy and proliferation), the release of GFAP PFs can indicate astroglia cell injury. Thus, this example shows that biofluid-based monitoring of the GFAP-released PFs can be used to monitor astroglia injury mediated by calpain activation.

### Example 11

### Calpain Digestion of Tau-441

Table 11 shows Tau PFs, generated by calpain digestion of Tau-441 protein and are found in ultrafiltrate samples. Tau-441 PFs generated by calpain digestion (mimicking TBI) include Tau N-terminal region peptide 1 AEPRQEFEVMEDHAGTYGLG (aa 2-21; SEQ ID NO:249); Tau N-terminal region peptide 2AAQPHTEIPEGTTAEEAGIGDTPSLEDEAAGHVTQARMVS (aa 90-123; SEQ NO:250); Tau center region peptide LSKVTSKCGSLG (aa 315-326; SEQ ID NO:251); Tau C-terminal region peptide 1 SPRHLSNVSSTGSIDMVDSPQLA (aa 404-426; SEQ ID NO:252); and Tau C-terminal region peptide 2 TLADEVSASLAKQGL (aa 427-441; SEQ ID NO:253). Table 11 lists further PFs along with MS/MS data for PFs found in TBI subject CSF ultrafiltrate samples or derived from in vitro calpain digestion of Tau and phospho-Tau protein (Tau-441; a model that mimics CNS traumatic injury-induced calpain activation).

The sequence of human Tau-441 (microtubule-associated protein Tau isoform 2; P10636-8) is:

SEQ ID NO: 254
MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMV DSPQLATLADEVSASLAKQGL.

The Tau PFs identified are shown in bold. Key Tau PFs identified here are shown in Table 12, below.

TABLE 12
Human Tau-441 Peptide Fragments (released by calpain digestion) as Identified by LC-MS/MS.

| SEQ | | proteolysis | Positions | | | Theo. | | | | |
|---|---|---|---|---|---|---|---|---|---|---|
| ID | | of Tau or | in | Protein | | MH+ | | m/z | ΔM | |
| NO | Annotated Sequence | P-Tau | Proteins | MOdifications | PSMs | (Da) | Charge | (Da) | (ppm) | XCorr |
| 255 | AEPRQEFEVMEDHA | Tau | 2-19 | none | 65 | 2065.89 | 3 | 689.625 | 468.96 | 4.71 |
| | GTYG | | | | | | | | | |
| 256 | KPVDLSKVTSKCG | P-Tau | 311-323 | none | 2 | 1361.746 | 2 | 681.415 | 56.61 | 3.44 |
| 257 | LSKVTSKCGSLG | Tau | 315-326 | none | 46 | 1179.64 | 1 | 1179.6 | −31.01 | 3.73 |
| 258 | RENAKAKTDHGAEI | Tau | 379-403 | none | 26 | 2672.36 | 3 | 891.934 | 533.08 | 5.3 |
| | VYKSPVVSGDT | | | | | | | | | |
| 259 | KSPVVSGDTSPRHLS | P-Tau | 395-412 | P10636-8 | 17 | 2106.866 | 2 | 1054.207 | 256.77 | 5.4 |
| | NVS | | | 3xPhospho | | | | | | |
| | | | | [S396(100); | | | | | | |
| | | | | S400(100); | | | | | | |
| | | | | S404(88.9)] | | | | | | |
| 260 | KSPVVSGDTSPRHLS | P-Tau | 395-426 | P10636-8 | 2 | 3508.51 | 3 | 1170.501 | 278.43 | 5.01 |
| | NVSSTGSIDMVDSPQ | | | 3xPhospho | | | | | | |
| | LA | | | [S396(99)] | | | | | | |
| 261 | SPRHLSNVSSTGSID | Tau | 404-426 | none | 39 | 2414.16 | 2 | 1208.29 | 582.72 | 5.95 |
| | MVDSPQLA | | | | | | | | | |
| 262 | STGSIDMVDSPQLA | P-Tau | 413-426 | P10636-8 | 40 | 1500.629 | 2 | 751.0989 | 374.32 | 4.18 |
| | | | | 1xPhospho | | | | | | |
| | | | | [S416(99.9)] | | | | | | |
| 263 | STGSIDMVDSPQLA | P-Tau | 413-426 | none | 37 | 1420.662 | 2 | 711.1865 | 495.08 | 4.2 |
| 264 | TLADEVSASLAKQG | Tau | 427-441 | none | 37 | 1502.81 | 3 | 502.358 | 1498.97 | 4.47 |
| | L.[-] | | | | | | | | | |
| 265 | ASLAKQGL.[-] | Tau | 434-441 | none | 32 | 787.467 | 2 | 394.375 | 349.85 | 2.51 |

FIG. 13A is a schematic representation of the Tau PFs generated by calpain digestion of Tau-441 protein ultrafiltrate samples, and shows Tau PFs, including AEPRQEFEVMEDHAGTYGLG (“Tau N-terminal peptide 1”; aa 2-21; SEQ ID NO:266) and TLADEVSASLAKQGL (“Tau C-terminal peptide 2”; 427-441; SEQ ID NO:267). FIG. 13B and FIG. 13C provide MS/MS spectra for these sequences. Tables 13 and 14, below present the identified b- and y-type ions for these peptides. Peptide ions in italics and underlined are found in MS/MS spectra.

TABLE 13
MS/MS Data for FIG. 13B.

| | | | | SEQ ID | | | | |
|---|---|---|---|---|---|---|---|---|
| #1 | b⁺ | b²⁺ | b³⁺ | NO:268 | y⁺ | y²⁺ | y³⁺ | #2 |
| 1 | 72.04439 | 36.52593 | 24.68631 | A | | | | 18 |
| 2 | 201.08698 | 101.04713 | 67.70051 | E | 1994.85488 | 997.93108 | 665.62314 | 17 |
| 3 | 298.13975 | 149.57351 | 100.05143 | P | 1865.81229 | 933.40978 | 622.60895 | 16 |
| 4 | 454.24086 | 227.62407 | 152.08514 | R | 1768.75952 | 884.88340 | 590.25803 | 15 |
| 5 | 582.29944 | 291.65336 | 194.77133 | Q | 1612.65841 | 806.83284 | 538.22432 | 14 |
| 6 | 711.34203 | 356.17465 | 237.78553 | E | 1484.59984 | 742.80356 | 495.53813 | 13 |
| 7 | 858.41044 | 429.70886 | 286.80833 | F | 1355.55724 | 678.28226 | 452.52393 | 12 |
| 8 | 987.45304 | 494.23019 | 329.82253 | E | 1208.48883 | 604.74805 | 403.50113 | 11 |
| 9 | 1086.52145 | 543.76436 | 362.84533 | V | 1079.44624 | 540.22676 | 360.48693 | 10 |
| 10 | 1217.56193 | 609.28461 | 406.52550 | M | 980.37782 | 490.69255 | 327.46412 | 9 |
| 11 | 1346.60453 | 673.80590 | 449.53969 | E | 849.33734 | 425.17231 | 283.78396 | 8 |
| 12 | 1461.63147 | 731.31937 | 487.88201 | D | 720.29474 | 360.65101 | 240.76977 | 7 |
| 13 | 1598.69038 | 799.84883 | 533.56831 | H | 605.26780 | 303.13754 | 202.42745 | 6 |
| 14 | 1669.72750 | 835.36739 | 557.24735 | A | 468.20889 | 234.60808 | 156.74115 | 5 |
| 15 | 1726.74896 | 86.87812 | 576.25450 | G | 397.17178 | 199.08953 | 133.06211 | 4 |
| 16 | 1827.79664 | 914.40196 | 609.93706 | T | 340.15031 | 170.57879 | 114.05496 | 3 |
| 17 | 1990.85997 | 995.93362 | 664.29151 | Y | 239.10263 | 120.05496 | 80.37240 | 2 |
| 18 | | | | G | 76.03930 | 38.52329 | 26.01795 | 1 |

TABLE 14
MS/MS Data for FIG. 13C.

| | | | SEQ ID | | | |
|---|---|---|---|---|---|---|
| #1 | b⁺ | b²⁺ | NO:269 | y⁺ | y²⁺ | #2 |
| 1 | 102.05496 | 51.53112 | T | | | 15 |
| 2 | 215.13902 | 108.07315 | L | 1401.75838 | 701.38283 | 14 |
| 3 | 286.17613 | 143.59170 | A | 1288.67432 | 644.84080 | 13 |
| 4 | 401.20308 | 201.10518 | D | 1217.63721 | 609.32224 | 12 |
| 5 | 530.24567 | 265.62647 | E | 1102.61026 | 551.80877 | 11 |
| 6 | 629.31408 | 315.16068 | V | 973.56767 | 487.28747 | 10 |
| 7 | 716.34611 | 358.67699 | S | 874.49926 | 437.75327 | 9 |
| 8 | 787.38322 | 394.19525 | A | 787.46723 | 394.23725 | 8 |
| 9 | 874.41525 | 437.71126 | S | 716.43012 | 358.71870 | 7 |
| 10 | 987.49932 | 494.25330 | L | 629.39809 | 315.20268 | 6 |
| 11 | 1058.53643 | 529.77185 | A | 516.31402 | 258.66065 | 5 |
| 12 | 1186.63139 | 593.81934 | K | 445.27691 | 223.14209 | 4 |
| 13 | 1314.68997 | 657.84862 | Q | 317.18195 | 159.09461 | 3 |
| 14 | 1371.71143 | 686.35936 | G | 189.12337 | 95.06532 | 2 |
| 15 | | | L | 132.10191 | 66.55459 | 1 |

FIG. 13D is a western blot showing calpain digestion of human tau-441 protein (63K) producing high molecular weight PBPs of 40-38K.

### Example 12

### Summary Chart of Additional Cortical and Hippocampus Fragments

Table 15, below shows the origin of PBP and PF biomarkers derived from additional proteins in mouse cortex or hippocampal ultrafiltrate samples after TBI (day 1 to day 3 post-injury. This example further supports use of biofluid-based monitoring of either specific brain protein PBPs or their unique PFs to inform on different brain vulnerabilities after brain injury (i.e., axonal marker astroglia, myelin and presynaptic terminal damage, respectively).

TABLE 15
Representative Peptide Fragments Identified from Mouse CCI (TBI) Cortex or
Hippocampal Ultrafiltrate Samples.

| | Full Protein Name | | | | | | | | | | |
|---|---|---|---|---|---|---|---|---|---|---|---|
| | (Mouse Gene Name) | | | Protein | | | | | | | |
| | Mouse Accession | Sequence | Number of PMSs/ | Group | | | | MH+ | ΔM | m/z | RT |
| SEQ ID NO | Number | (Amino Acid Residues) | Proteins/Groups | Accesions | Modifications | XCorr | Charge | [Da] | [ppm] | [Da] | [min] |
| 270 | Amyloid beta A4 | GAPGNSEQDFMSD | 1/1/1 | Q8R5A3 | | 2.05 | 3 | 1355.64 | 189.12 | 452.55 | 55.71 |
| | precursor protein- | (645-656) | | | | | | | | | |
| | binding family B | | | | | | | | | | |
| | member 1-interacting | | | | | | | | | | |
| | protein (Apbb1ip) | | | | | | | | | | |
| | Q8R5A3 | | | | | | | | | | |
| 271 | Calmodulin-regulated | IQALAQKGLY | 1/3/1 | H7BX08 | | 2.36 | 3 | 1105.93 | 544.05 | 369.31 | 14.96 |
| | spectrin-associated | (104-115) | | | | | | | | | |
| | protein 2 (Camsap2) | | | | | | | | | | |
| | H7BX08 | | | | | | | | | | |
| 272 | Calmodulin-regulated | MGAHLAVI | 1/3/1 | E9Q5B0 | | 2.41 | 2 | 812.61 | 720.42 | 406.81 | 14.98 |
| | spectrin-associated | (134-141) | | | | | | | | | |
| | protein 3 (Camsap3) | | | | | | | | | | |
| | E9Q5B0 | | | | | | | | | | |
| 273 | Chondroitin sulfate | FFGENHLEVPVPSAL | 1/2/1 | Q8VHY0 | | 2.10 | 3 | 3372.22 | −173.21 | 1124.75 | 70.85 |
| | proteoglycan 4 | TRVDLLLQFSTSQPE | | | | | | | | | |
| | (Cspg4) Q8VHY0 | (32-61) | | | | | | | | | |
| 274 | Disks large homolog 2, | TmPSSGPGGPAS | 2/2/1 | Q91XM9-4 | M2 (Oxidation) | 2.20 | 2 | 1061.62 | −494.62 | 531.31 | 99.75 |
| | isoform 4, PSD93-delta | (50-61) | | | | | | | | | |
| | (Dlg2) Q91XM9 | | | | | | | | | | |
| 275 | Glial fibrillary acidic | EERHARESASYQEAL | 1/2/1 | P03995-2 | | 2.55 | 3 | 4548.34 | −116.66 | 1516.79 | 90.67 |
| | protein (Gfap) P03995-2 | ARLEEEGQSLKEEMA | | | | | | | | | |
| | | RHLQEYQD | | | | | | | | | |
| | | (311-348) | | | | | | | | | |
| 276 | Glutamate | RVAPKIKALMMESG | 1/1/1 | P48318 | M32(Oxidation) | 2.32 | 3 | 3834.34 | 189.17 | 1278.78 | 76.00 |
| | decarboxylase 1 | TTMVGYQPQGDKAN | | | | | | | | | |
| | (Gad1) P48318 | FFRmVI (536-579) | | | | | | | | | |
| 277 | Glutamate | LEYVTLKK | 1/1/1 | P48320 | | 2.21 | 2 | 994.66 | 442.34 | 497.83 | 15.83 |
| | decarboxylase 2 | (215-223) | | | | | | | | | |
| | (Gad2) P48320 | | | | | | | | | | |
| 278 | Huntingtin (Htt) | QQVKDTSLKGSFGVT | 1/3/1 | G3X9115 | M19 (Oxidation) | 2.23 | 3 | 2155.76 | −331.23 | 719.26 | 43.38 |
| | G3X9H5 | RKEm (306-325) | | | | | | | | | |
| 279 | Neurochondrin (Ncdn) | LKEPQKVQLVSIMKE | 1/2/1 | Q9Z0E0-2 | | 2.24 | 3 | 1955.92 | 258.88 | 652.65 | 53.22 |
| | Q9Z0E0-2 | AI (339-355) | | | | | | | | | |
| 280 | Macrophage mannose | DEQVQFTHWNADMP | 1/1/1 | Q61830 | C20 | 2.21 | 3 | 4352.66 | −56.09 | 1451.56 | 67.94 |
| | receptor 1 (MRC1, | GRKAGcVAMKTGVA | | | (Carbamidomethyl); | | | | | | |
| | CD206)(Mrc1) | GGLWDVLScEE (581- | | | C37 | | | | | | |
| | Q61830 | 619) | | | (Carbamidomethyl) | | | | | | |
| 281 | Microtubule-associated | IQAEPLYRVVSNTIEP | 1/3/1 | A2ARP8 | M30 (Oxidation) | 2.12 | 3 | 4979.38 | 102.20 | 1660.46 | 96.18 |
| | protein 1A (Map1a) | LTLFHKMGVGRLDm | | | | | | | | | |
| | A2ARP8 | YVLNPVKDSKEMQ | | | | | | | | | |
| | | (409-451) | | | | | | | | | |
| 282 | Microtubule-associated | NASASKSAKTATAGP | 1/1/1 | P14873 | C37 | 2.06 | 3 | 3650.08 | −19.20 | 1217.36 | 65.84 |
| | protein 1B (Map1b) | GTTKTAKSSTVPPGL | | | (Carbamidomethyl) | | | | | | |
| | P14873 | PVYLDLc (2340-2357) | | | | | | | | | |
| 283 | Microtubule-associated | SPGPLTPMREKDVLE | 1/4/1 | P20357 | | 2.27 | 3 | 3639.09 | −16.87 | 1213.70 | 83.79 |
| | protein 2 (Map2) | DIPRWEGKQFDSPMP | | | | | | | | | |
| | P20357 | SP (283-314) | | | | | | | | | |
| 284 | Microtubule-associated | SVDRETVAAPGRSGL | 1/3/1 | Q7TSJ2 | | 2.00 | 3 | 2616.14 | −255.48 | 872.72 | 60.50 |
| | protein 6 (Map6) | GLGAASASTSGSGP | | | | | | | | | |
| | Q7TSJ2 | (86-114) | | | | | | | | | |
| 285 | Myelin A1 protein | HYGSLPQKSQ | 1/17/2 | P04370; | | 2.70 | 2 | 1145.83 | 499.19 | 573.42 | 10.27 |
| | (GOLI-MBP) | (198-207) | | F6VME3 | | | | | | | |
| | (Mbp(A1)) P04370 | | | | | | | | | | |
| 286 | Myelin basic protein | KNIVTPRTPPPSQGK | 1/15/4 | P04370; | | 3.78 | 3 | 1677.61 | −202.90 | 559.87 | 10.10 |
| | isoform 4 (MBP (4)) | G (114-130) | | F6RT34; | | | | | | | |
| | P04370-4 | | | F6TYB7; | | | | | | | |
| | | | | F7A0B0 | | | | | | | |
| 287 | Myelin regulatory | RKHSESPPNTLN | 1/4/1 | Q3UR85 | | 2.07 | 3 | 1380.40 | −74.32 | 460.81 | 43.81 |
| | factor (MYRF) | (256-267) | | | | | | | | | |
| | Q3UR85 | | | | | | | | | | |
| 288 | Neurogranin (NRGN) | AAKIQASF (27-37) | 2/1/1 | P60761 | | 2.89 | 2 | 836.53 | 664.08 | 418.77 | 26.19 |
| | P60761 | | | | | | | | | | |
| 289 | Neurexin-1 (Nrxn1) | RLVGEVPSSmTTEST | 1/10/2 | E0CY11; | M10 (Oxidation) | 2.32 | 3 | 1854.09 | 21.19 | 618.70 | 98.25 |
| | E0CY11 | ATA (1313-1330) | | P0DI97 | | | | | | | |
| 290 | Neurexin-3, isoform | IMTEKRYISVVPSSFI | 1/3/1 | Q6P9K9 | M23 (Oxidation); | 2.28 | 3 | 4830.21 | 116.74 | 1610.74 | 77.78 |
| | 2a (Nrxn3) Q6P9K9-2 | GHLQSLmFNGLLYID | | | C33 | | | | | | |
| | | LcKNGDIDYc (444-484) | | | (Carbamidomethyl); | | | | | | |
| | | | | | C41 | | | | | | |
| | | | | | (Carbamidomethyl) | | | | | | |
| 291 | Neurocan core protein | TIAAPVEASHRSPDA | 1/1/1 | P55066 | | 1.99 | 3 | 4377.09 | −161.54 | 1459.70 | 73.06 |
| | (Ncan) P55066 | DSIEIEGTSSMRATKH | | | | | | | | | |
| | | PISGPWASLDS (699- | | | | | | | | | |
| | | 740) | | | | | | | | | |
| 292 | Neurofascin (Nfasc) | DIYSARGVAERTPSF | 1/1/1 | E9PW06 | C37 | 1.50 | 3 | 4352.19 | −194.02 | 1451.40 | 70.08 |
| | E9PW06 | MYPQGTSSSQMVLR | | | (Carbamidomethyl) | | | | | | |
| | | GMDLLLEcIA (243- | | | | | | | | | |
| | | 281) | | | | | | | | | |
| 293 | Neurofilament heavy | KmEAKVKEDDKSLS | 1/1/1 | P19246 | M2 (Oxidation) | 2.16 | 3 | 3469.62 | 204.48 | 1157.21 | 63.22 |
| | polypeptide (Neth) | KEPSKPKTEKAEKSS | | | | | | | | | |
| | P19246 | ST (1039-1069) | | | | | | | | | |
| 294 | Neurofilament light | YSQSSQVFGRSAYSG | 1/1/1 | P08551 | M23 (Oxidation) | 2.09 | 2 | 3918.73 | −129.67 | 1959.87 | 83.77 |
| | polypeptide (Nefl) | LQSSSYLmSARSFPA | | | | | | | | | |
| | P08551 | YYTSH (413-447) | | | | | | | | | |
| 295 | Secretogranin-2 (Scg) | IPVGSLKNEDTPN | 1/1/1 | Q03517 | | 3.07 | 2 | 1385.27 | 537.32 | 693.14 | 24.77 |
| | Q03517 | (569-581) | | | | | | | | | |
| 296 | Spectrin alpha chain, | LIERGAcAGSEDAVK | 1/5/1 | A3KGU5 | C7 | 2.07 | 3 | 4046.98 | −149.39 | 1349.67 | 63.35 |
| | non-erythrocytic 1 | ARLAALADQWQFLV | | | (Carbamidomethyl) | | | | | | |
| | (Sptan1) A3KGU5 | QKSAEKSQ (601- | | | | | | | | | |
| | | 1637) | | | | | | | | | |
| 297 | Spectrin beta chain, | TLEGAEAAIKKQEDF | 1/2/1 | Q62261-2 | M19 (Oxidation) | 2.11 | 3 | 3914.52 | 39.15 | 1305.51 | 67.94 |
| | non-erythrocytic 1 | MTTmDANEEKINAV | | | | | | | | | |
| | (isoform 2)(Sptbn1) | VETGRR (1187-1221) | | | | | | | | | |
| | Q62261 | | | | | | | | | | |
| 298 | Synapsin-2 (isoform | MTDLQRPEPQQPPPA | 1/2/1 | Q64332-2 | | 2.15 | 3 | 3511.49 | −104.38 | 1171.17 | 90.86 |
| | IIb)(Syn)Q64332-2 | PGPGAATASAATSAA | | | | | | | | | |
| | | SPGPER (23-58) | | | | | | | | | |
| 299 | Synaptotagmin-2 | YDKLGKNEAIGKI | 1/1/1 | P46097 | | 2.19 | 3 | 1449.18 | −354.60 | 483.73 | 22.23 |
| | (Syt2) P46097 | (364-377) | | | | | | | | | |
| 300 | Tubulin beta-4A chain | KNmmAAcDPRHGRY | 1/1/1 | Q9D6F9 | M3 (Oxidation); M4 | 2.13 | 3 | 4253.07 | 24.02 | 1418.36 | 73.26 |
| | (Tubb4a) Q9D6F9 | LTVAAVFRGRmSmK | | | (Oxidation); C7 | | | | | | |
| | | EVDEQMLS (297-332) | | | Carbamidomethyl); | | | | | | |
| | | | | | M25 (Oxidation); | | | | | | |
| | | | | | M27 (Oxidation) | | | | | | |
| 301 | Tubulin beta-4B chain | AmFRRKAFLHWYTG | 1/6/2 | P68372; | M2 (Oxidation); | | | | | | |
| | (Tubb4b) P68372 | EGmDEmEFTEAES | | Q9ERD7 | M17 (Oxidation); | | | | | | |
| | | (387-418) | | | M20 (Oxidation) | 2.13 | 2 | 3320.40 | 213.45 | 1660.70 | 90.45 |
| 302 | Vimentin P20152-1 | VSSSSYRRMFGGSGT | 1/1/1 | P20152 | M9 (Oxidation) | 2.16 | 3 | 2553.19 | 330.04 | 851.06 | 15.11 |
| | | SSRPSSNRS (06-27) | | | | | | | | | |

### Example 13

### Identification of Neurogranin Peptide

FIG. 14A shows neurogranin proteolytic peptide ILDIPLDDPGANAAAAKIQAS (p)FRGHMARKKIKSGERGRKGPGPGGPGGA (amino acid residues 16-64; SEQ ID NO:303), identified in a biofluid (CSF) sample from a human TBI subject less than or equal to 24 hours after TBI, but not found or in much low levels in control CSF sample. The NRGN peptide appears as a charge of +7, monoisotopic m/z 713.64 Da. Ser-36 was found to be phosphorylated (p). The spectrum shows the fragment ions with identified b+ and y+ type ions in italics and underline, respectively, in Table 16, below.

TABLE 16
MS/MS Data for FIG. 14A.

| | | | | | | | SEQ ID | | | | | | | |
|---|---|---|---|---|---|---|---|---|---|---|---|---|---|---|
| #1 | b⁺ | b²⁺ | b³⁺ | b⁴⁺ | b⁵⁺ | b⁶⁺ | NO:304 | y⁺ | y²⁺ | y³⁺ | y⁴⁺ | y⁵⁺ | y⁶⁺ | #2 |
| 1 | 114.09 | 57.55 | 38.70 | 29.28 | 23.62 | 19.85 | I | | | | | | | 49 |
| 2 | 227.18 | 114.09 | 76.40 | 57.55 | 46.24 | 38.70 | L | 4874.51 | 2437.76 | 1625.51 | 1219.38 | 975.71 | 813.26 | 48 |
| 3 | 342.20 | 171.60 | 114.74 | 86.31 | 69.25 | 57.87 | D | 4761.43 | 2381.22 | 1587.81 | 1191.11 | 953.09 | 794.41 | 47 |
| 4 | 455.29 | 228.15 | 152.43 | 114.58 | 91.86 | 76.72 | I | 4646.40 | 2323.70 | 1549.47 | 1162.36 | 930.09 | 775.24 | 46 |
| 5 | 552.34 | 276.67 | 184.78 | 138.84 | 111.27 | 92.90 | P | 4533.32 | 2267.16 | 1511.78 | 1134.08 | 907.47 | 756.39 | 45 |
| 6 | 665.42 | 333.22 | 222.48 | 167.11 | 133.89 | 111.74 | L | 4436.26 | 2218.64 | 1479.43 | 1109.82 | 888.06 | 740.22 | 44 |
| 7 | 780.45 | 390.73 | 260.82 | 195.87 | 156.90 | 130.91 | D | 4323.18 | 2162.09 | 1441.73 | 1081.55 | 865.44 | 721.37 | 43 |
| 8 | 895.48 | 448.24 | 299.16 | 224.62 | 179.90 | 150.09 | D | 4208.15 | 2104.58 | 1403.39 | 1052.79 | 842.44 | 702.20 | 42 |
| 9 | 992.53 | 496.77 | 331.51 | 248.89 | 199.31 | 166.26 | P | 4093.13 | 2047.07 | 1365.05 | 1024.04 | 819.43 | 683.03 | 41 |
| 10 | 1049.55 | 525.28 | 350.52 | 263.14 | 210.72 | 175.76 | G | 3996.07 | 1998.54 | 1332.70 | 999.77 | 800.02 | 666.85 | 40 |
| 11 | 1120.59 | 560.80 | 374.20 | 280.90 | 224.92 | 187.60 | A | 3939.05 | 1970.03 | 1313.69 | 985.52 | 788.62 | 657.35 | 39 |
| 12 | 1234.63 | 617.82 | 412.22 | 309.41 | 247.73 | 206.61 | N | 3868.01 | 1934.51 | 1290.01 | 967.76 | 774.41 | 645.51 | 38 |
| 13 | 1305.67 | 653.34 | 435.89 | 327.17 | 261.94 | 218.45 | A | 3753.97 | 1877.49 | 1252.00 | 939.25 | 751.60 | 626.50 | 37 |
| 14 | 1376.71 | 688.86 | 459.57 | 344.93 | 276.15 | 230.29 | A | 3682.93 | 1841.97 | 1228.32 | 921.49 | 737.39 | 614.66 | 36 |
| 15 | 1447.74 | 724.38 | 483.25 | 362.69 | 290.35 | 242.13 | A | 3611.90 | 1806.45 | 1204.64 | 903.73 | 723.19 | 602.82 | 35 |
| 16 | 1518.78 | 759.89 | 506.93 | 380.45 | 304.56 | 253.97 | A | 3540.86 | 1770.93 | 1180.96 | 885.97 | 708.98 | 590.98 | 34 |
| 17 | 1646.87 | 823.94 | 549.63 | 412.47 | 330.18 | 275.32 | K | 3469.82 | 1735.41 | 1157.28 | 868.21 | 694.77 | 579.14 | 33 |
| 18 | 1759.96 | 880.48 | 587.32 | 440.75 | 352.80 | 294.17 | I | 3341.73 | 1671.37 | 1114.58 | 836.19 | 669.15 | 557.79 | 32 |
| 19 | 1888.02 | 944.51 | 630.01 | 472.76 | 378.41 | 315.51 | Q | 3228.64 | 1614.83 | 1076.89 | 807.92 | 646.53 | 538.95 | 31 |
| 20 | 1959.05 | 980.03 | 653.69 | 490.52 | 392.62 | 327.35 | A | 3100.58 | 1550.80 | 1034.20 | 775.90 | 620.92 | 517.60 | 30 |
| 21 | 2126.05 | 1063.53 | 709.36 | 532.27 | 426.02 | 355.18 | S (p) | 3029.55 | 1515.28 | 1010.52 | 758.14 | 606.72 | 505.76 | 29 |
| 22 | 2273.12 | 1137.06 | 758.38 | 569.04 | 455.43 | 379.69 | F | 2862.55 | 1431.78 | 954.85 | 716.39 | 573.32 | 477.93 | 28 |
| 23 | 2429.22 | 1215.11 | 810.41 | 608.06 | 486.65 | 405.71 | R | 2715.48 | 1358.24 | 905.83 | 679.63 | 543.90 | 453.42 | 27 |
| 24 | 2486.24 | 1243.63 | 829.42 | 622.32 | 498.05 | 415.21 | G | 2559.38 | 1280.19 | 853.80 | 640.60 | 512.68 | 427.40 | 26 |
| 25 | 2623.30 | 1312.16 | 875.11 | 656.58 | 525.47 | 438.06 | H | 2502.36 | 1251.68 | 834.79 | 626.35 | 501.28 | 417.90 | 25 |
| 26 | 2770.34 | 1385.67 | 924.12 | 693.34 | 554.87 | 462.56 | M (ox) | 2365.30 | 1183.15 | 789.10 | 592.08 | 473.87 | 395.06 | 24 |
| 27 | 2841.38 | 1421.19 | 947.80 | 711.10 | 569.08 | 474.40 | A | 2218.26 | 1109.64 | 740.09 | 555.32 | 444.46 | 370.55 | 23 |
| 28 | 2997.48 | 1499.24 | 999.83 | 750.12 | 600.30 | 500.42 | R | 2147.23 | 1074.12 | 716.41 | 537.56 | 430.25 | 358.71 | 22 |
| 29 | 3125.57 | 1563.29 | 1042.53 | 782.15 | 625.92 | 521.77 | K | 1991.13 | 996.07 | 664.38 | 498.54 | 399.03 | 332.69 | 21 |
| 30 | 3253.67 | 1627.34 | 1085.23 | 814.17 | 651.54 | 543.12 | K | 1863.03 | 932.02 | 621.68 | 466.51 | 373.41 | 311.34 | 20 |
| 31 | 3366.75 | 1683.88 | 1122.92 | 842.44 | 674.16 | 561.96 | I | 1734.94 | 867.97 | 578.98 | 434.49 | 347.79 | 290.00 | 19 |
| 32 | 3494.85 | 1747.93 | 1165.62 | 874.47 | 699.77 | 583.31 | K | 1621.85 | 811.43 | 541.29 | 406.22 | 325.18 | 271.15 | 18 |
| 33 | 3581.88 | 1791.44 | 1194.63 | 896.22 | 717.18 | 597.82 | S | 1493.76 | 747.38 | 498.59 | 374.19 | 299.56 | 249.80 | 17 |
| 34 | 3638.90 | 1819.95 | 1213.64 | 910.48 | 728.59 | 607.32 | G | 1406.72 | 703.87 | 469.58 | 352.44 | 282.15 | 235.29 | 16 |
| 35 | 3767.94 | 1884.47 | 1256.65 | 942.74 | 754.39 | 628.83 | E | 1349.70 | 675.36 | 450.57 | 338.18 | 270.75 | 225.79 | 15 |
| 36 | 3924.04 | 1962.52 | 1308.69 | 981.77 | 785.61 | 654.85 | R | 1220.66 | 610.83 | 407.56 | 305.92 | 244.94 | 204.28 | 14 |
| 37 | 3981.06 | 1991.04 | 1327.69 | 996.02 | 797.02 | 664.35 | G | 1064.56 | 532.78 | 355.52 | 266.90 | 213.72 | 178.27 | 13 |
| 38 | 4137.17 | 2069.09 | 1379.73 | 1035.05 | 828.24 | 690.37 | R | 1007.54 | 504.27 | 336.52 | 252.64 | 202.31 | 168.76 | 12 |
| 39 | 4265.26 | 2133.13 | 1422.42 | 1067.07 | 853.86 | 711.72 | K | 851.44 | 426.22 | 284.48 | 213.61 | 171.09 | 142.75 | 11 |
| 40 | 4322.28 | 2161.64 | 1441.43 | 1081.33 | 865.26 | 721.22 | G | 723.34 | 362.17 | 241.79 | 181.59 | 145.47 | 121.40 | 10 |
| 41 | 4419.33 | 2210.17 | 1473.78 | 1105.59 | 884.67 | 737.40 | P | 666.32 | 333.66 | 222.78 | 167.34 | 134.07 | 111.89 | 9 |
| 42 | 4476.36 | 2238.68 | 1492.79 | 1119.84 | 896.08 | 746.90 | G | 569.27 | 285.14 | 190.43 | 143.07 | 114.66 | 95.72 | 8 |
| 43 | 4573.41 | 2287.21 | 1525.14 | 1144.11 | 915.49 | 763.07 | P | 512.25 | 256.63 | 171.42 | 128.82 | 103.26 | 86.21 | 7 |
| 44 | 4630.43 | 2315.72 | 1544.15 | 1158.36 | 926.89 | 772.58 | G | 415.19 | 208.10 | 139.07 | 104.55 | 83.84 | 70.04 | 6 |
| 45 | 4687.45 | 2344.23 | 1563.16 | 1172.62 | 938.30 | 782.08 | G | 358.17 | 179.59 | 120.06 | 90.30 | 72.44 | 60.53 | 5 |
| 46 | 4784.50 | 2392.76 | 1595.51 | 1196.88 | 957.71 | 798.26 | P | 301.15 | 151.08 | 101.06 | 76.04 | 61.04 | 51.03 | 4 |
| 47 | 4841.53 | 2421.27 | 1614.51 | 1211.14 | 969.11 | 807.76 | G | 204.10 | 102.55 | 68.70 | 51.78 | 41.63 | 34.86 | 3 |
| 48 | 4898.55 | 2449.78 | 1633.52 | 1225.39 | 980.52 | 817.26 | G | 147.08 | 74.04 | 49.70 | 37.52 | 30.22 | 25.35 | 2 |
| 49 | | | | | | | A | 90.05 | 45.53 | 30.69 | 23.27 | 18.82 | 15.85 | 1 |

The NGRN PFs included those listed in Table 17, below. The full sequence of NRGN (78 amino acids) is

SEQ ID NO: 305
MDCCTENACSKPDDDILDIPLDDPGANAAAAKIQASFRGHMARKKI
KSGERGRKGPGPGGPGGAGVARGGAGGGPSGD.

Underlined residues show the area in the sequence where PFs are produced.

FIG. 14B also shows MS/MS label-free quantification of the unique phospho-NRGN peptide (aa 16-64) in TBI (<24 h) (n=30) vs Control CSF samples (n=10)

Table 17 is a representation showing the NRGN-derived PFs generated and released into CSF from human TBI subjects. Duplicate PFs found are not shown. None of the PFs shown was found in non-injured control CSF samples. FIG. 14C shows schematic representation for the NRGN peptides generated and released into human CSF samples (n=30) after TBI. Duplicate peptides are not shown. None of the peptides shown was found in non-injured control CSF samples (n=10).

TABLE 17
Neurogranin-Derived Peptide Fragments Released after TBI in Human Subjects.

| | | Amino Acid | SEQ |
|---|---|---|---|
| | | Residues of | ID |
| Name | Sequence | Neurogranin | NO |
| Neurogranin | ILDIPLDDPGANAAAAKIQASFRGHMARKKIKSGERGRK | 18-64 | 306 |
| Peptide 1 | GPGPGGPGGA | | |
| Neurogranin | ILDIPLDDPGANAAAAKIQAS(p)FRGHMARKKIKSGERGR | 18-64 | 307 |
| Peptide 2 | KGPGPGGPGGA | | |
| Neurogranin | DDDILDIPLDDPGANAAAAKIQASFR | 13-38 | 308 |
| Peptide 3 | | | |
| Neurogranin | DDDILDIPLDDPGANAAAAKIQAS(p)FR | 13-38 | 309 |
| Peptide 4 | | | |
| Neurogranin | PGANAAAAKIQASFRGHMARKKIKSGERGRKGPGPGG | 24-65 | 310 |
| Peptide 5 | | | |
| Neurogranin | PGANAAAAKIQAS(p)FRGHMARKKIKSGERGRKGPGPGG | 24-65 | 311 |
| Peptide 6 | | | |

FIG. 14D shows quantitative immunblotting evidence that human CSF profile of NRGN PBP released less than or equal to 24 hours after TBI in CSF compared to controls. The blots were probed with an internal NRGN epitope antibody (EMD AB5620). An equal CSF volume was loaded to mimic the ELISA-based diagnostic test where biomarker levels are reported as pg or ng per mL. Also, for a positive control, the blot concurrently was probed with αII-spectrin antibody (mAb). The intact αII-spectrin (260 kDa) and its major fragments SBDP150 and SBDP145 were observed in most TBI CSF samples.

FIG. 14E shows densitometric quantitation of intact NRGN and its PBP/PF (P-NRGN-BDP), shown as a scattered plot with mean and SEM. * indicates statistical significance over naive (p-value <0.05, 2 tailed unpaired T-test). FIG. 14F shows diagnostic Receiver operating characteristic curve (ROC) curves of intact NRGN and P-NRGN-BDP comparing Control CSF (N=10) vs. TBI CSF. (N=30). Each ROC curve's, area under the curve, SEM, 95% confidence interval and P value are shown under the curve, respectively. NRGN-BDP shows a superior diagnostic property with ROC ACU of 0.956 verssus intact NRGN AUC of only 0.815. As NRGN is a key component of the postsynaptic terminal, the levels of NRGN PFs or PBPs in biofluid reflects the extent of postsynaptic terminal damage. Thus, this example shows that human biofluid-based monitoring of PFs of NRGN can be used to monitor postsynaptic terminal damage.

### Example 14

### Vimentin Peptide Fragments and Vimentin-PBP in CSF from Human TBI Subjects

VIM PFs Identified from human TBI subjects also were characterized. FIG. 15 shows data relating to VIM PBP or PF in CSF from human TBI subjects less than or equal to 24 hours after TBI. FIG. 15A is an MS/MS spectrum of the VIM peptide NVKMALDIEIAT(p) (amino acids 388-399; SEQ ID NO:312), charge+2, monoisotopic m/z 699.34711 Da. The spectrum shows the fragment ions with identified b+ and y+ type ions in italics and underline, respectively, in Table 18, below. Thr-399 was found to be phosphorylated (p).

TABLE 18
MS/MS Data for FIG. 15A.

| | | | SEQ ID | | | |
|---|---|---|---|---|---|---|
| #1 | b⁺ | b²⁺ | NO:313 | y⁺ | y²⁺ | #2 |
| 1 | 115.05 | 58.03 | N | | | 12 |
| 2 | 214.12 | 107.56 | V | 1283.63 | 642.32 | 11 |
| 3 | 342.21 | 171.61 | K | 1184.56 | 592.79 | 10 |
| 4 | 473.25 | 237.13 | M | 1056.47 | 528.74 | 9 |
| 5 | 544.29 | 272.65 | A | 925.43 | 463.22 | 8 |
| 6 | 657.38 | 329.19 | L | 854.39 | 427.70 | 7 |
| 7 | 772.40 | 386.70 | D | 741.31 | 371.16 | 6 |
| 8 | 885.49 | 443.25 | I | 626.28 | 313.64 | 5 |
| 9 | 1014.53 | 507.77 | E | 513.20 | 257.10 | 4 |
| 10 | 1127.61 | 564.31 | I | 384.15 | 192.58 | 3 |
| 11 | 1198.65 | 599.83 | A | 271.07 | 136.04 | 2 |
| 12 | | | T (p) | 200.03 | 100.52 | 1 |

FIG. 15B and Table 19, below, show the same type of data for another VIM peptide identified in human CSF (LLEGEESRISLPLPNFSSLNLR (amino acids 403-424; SEQ ID NO:314). The spectrum also shows the fragment ions with identified b+ and y+ type ions in italics and underline, respectively.

TABLE 19
MS/MS Data for FIG. 15B.

| | | | | | SEQ ID | | | | | |
|---|---|---|---|---|---|---|---|---|---|---|
| #1 | b⁺ | b²⁺ | b³⁺ | b⁴⁺ | NO:315 | y⁺ | y²⁺ | y³⁺ | y⁴⁺ | #2 |
| 1 | 114.09134 | 57.54931 | 38.70196 | 29.27829 | L | | | | | 22 |
| 2 | 227.17540 | 114.09134 | 76.39665 | 57.54931 | L | 2531.19426 | 1266.10077 | 844.40294 | 633.55402 | 21 |
| 3 | 356.21800 | 178.61264 | 119.41085 | 89.80996 | E | 2418.11020 | 1209.55874 | 806.70825 | 605.28301 | 20 |
| 4 | 413.23946 | 207.12337 | 138.41800 | 104.06532 | G | 2289.06760 | 1145.03744 | 763.69405 | 573.02236 | 19 |
| 5 | 542.28025 | 271.64467 | 181.43220 | 136.32597 | E | 2232.04614 | 1116.52671 | 744.68690 | 558.76699 | 18 |
| 6 | 671.32465 | 336.16596 | 224.44640 | 168.58662 | E | 2103.00355 | 1052.00541 | 701.67270 | 526.50634 | 17 |
| 7 | 758.35668 | 379.68198 | 253.45708 | 190.34463 | S | 1973.96095 | 987.48412 | 658.65850 | 494.24570 | 16 |
| 8 | 914.45779 | 457.73253 | 305.49078 | 229.36990 | R | 1886.92893 | 943.96810 | 629.64783 | 472.48769 | 15 |
| 9 | 1027.54185 | 514.27456 | 343.18547 | 257.64092 | I | 1730.82782 | 865.91755 | 577.61412 | 433.46241 | 14 |
| 10 | 1194.54021 | 597.77374 | 398.85159 | 299.39051 | S (p) | 1617.74375 | 809.37551 | 539.91943 | 405.19140 | 13 |
| 11 | 1307.62427 | 654.31578 | 436.54628 | 327.66153 | L | 1450.74539 | 725.87633 | 484.25332 | 363.44181 | 12 |
| 12 | 1404.67704 | 702.84216 | 468.89720 | 351.92472 | P | 1337.66133 | 669.33430 | 446.55863 | 335.17079 | 11 |
| 13 | 1517.76110 | 759.38419 | 506.59188 | 380.19573 | L | 1240.60856 | 620.80792 | 414.20771 | 310.90760 | 10 |
| 14 | 1614.81387 | 807.91057 | 538.94281 | 404.45892 | P | 1127.52450 | 564.26589 | 376.51302 | 282.63658 | 9 |
| 15 | 1728.85679 | 864.93203 | 576.95712 | 432.96966 | N | 1030.47174 | 515.73951 | 344.16210 | 258.37339 | 8 |
| 16 | 1875.92521 | 938.46624 | 625.97992 | 469.73676 | F | 916.42881 | 458.71804 | 306.14779 | 229.86266 | 7 |
| 17 | 2042.92357 | 1021.96542 | 681.64604 | 511.48635 | S (p) | 769.36040 | 385.18384 | 257.12498 | 193.09556 | 6 |
| 18 | 2129.95559 | 1065.48144 | 710.65672 | 533.24436 | S | 602.36204 | 301.68466 | 201.45886 | 151.34597 | 5 |
| 19 | 2243.03966 | 1122.02347 | 748.35140 | 561.51537 | L | 515.33001 | 258.16864 | 172.44819 | 129.58796 | 4 |
| 20 | 2357.08259 | 1179.04493 | 786.36571 | 590.02610 | N | 402.24594 | 201.62661 | 134.75350 | 101.31694 | 3 |
| 21 | 2470.16665 | 1235.58696 | 824.06040 | 618.29712 | L | 288.20302 | 144.60515 | 96.73919 | 72.80621 | 2 |
| 22 | | | | | R | 175.11895 | 88.06311 | 59.04450 | 44.53520 | 1 |

The amino acid sequence of human VIM (accession #P08670) is:

SEQ ID NO: 316
MSTRSVSSSSYRRMFGGPGTASRPSSSRSYVTTSTRTYSLGSALRP
STSRSLYASSPGGVYATRSSAVRLRSSVPGVRLLQDSVDFSLADAI
NTEFKNTRTNEKVELQELNDRFANYIDKVRFLEQQNKILLAELEQL
KGQGKSRLGDLYEEEMRELRRQVDQLTNDKARVEVERDNLAEDIMR
LREKLQEEMLQREEAENTLQSFRQDVDNASLARLDLERKVESLQEE
IAFLKKLHEEEIQELQAQIQEQHVQIDVDVSKPDLTAALRDVRQQY
ESVAAKNLQEAEEWYKSKFADLSEAANRNNDALRQAKQESTEYRRQ
VQSLTCEVDALKGTNESLERQMREMEENFAVEAANYQDTIGRLQDE
IQNMKEEMARHLREYQDLLNVKMALDIEIATYRKLLEGEESRISLP
LPNFSSLNLRETNLDSLPLVDTHSKRTLLIKTVETRDGQVINETSQ
HHDDLE.

Residues underlined and in bold show the areas which the VIM PFs are released.

FIG. 15C shows vimentin-PF characterization in CSF from human TBI subjects. (A) MS label free quantification of VIM-N and C-terminal proteolytic peptide fragments (as indicated) in TBI vs Control CSF samples mean and SEM are shown. * shows statistical significance over naïve (p-value <0.05, 2 tailed unpaired T-test).

Preferred PFs according to the invention include those listed in Table 20 below and in FIG. 15D.

TABLE 20
Vimentin-Derived Peptide Fragments Released after TBI in Human Subjects.

| | | Amino Acid | SEQ |
|---|---|---|---|
| | | Residues of | ID |
| Name | Sequence | Vimentin | NO |
| “Vimentin | NVKMALDIEIAT | 388-399 | 317 |
| C-terminal | | | |
| Peptide 1” | | | |
| ”Vimentin | LLEGEESRISLPLPNFSSLNLR | 403-424 | 318 |
| C-terminal | | | |
| Peptide 2” | | | |
| “Vimentin | NVKMALDIEIATYRKLLEGEESRISLPLPNFSSLNLRE | 388-456 | 319 |
| C-terminal | TNLDSLPLVDTHSKRTLLIKTVETRDGQVIN | | |
| Peptide 3” | | | |
| “Vimentin | MSTRSVSSSS YRRMFGGPGT ASRPSSSRSY | 1-75 | 320 |
| N-terminal | VTTSTRTYSL GSALRPSTSR SLYASSPGGV | | |
| Peptide 1” | YATRSSAVRL RSSVP | | |
| “Vimentin | STRSVSSSSYRRMFGGPGTASRPSSSRSYVTTSTRTY | 2-47 | 321 |
| N-terminal | SLGSALR | | |
| Peptide 2” | | | |

FIG. 15E shows a profile of human CSF VIM breakdown products (38 kDa and 26 kDa) released less than or equal to 24 hours after TBI in human subjects, compared to controls. The western blot was probed with an anti-VIM internal epitope antibody (Abcam ab92547) to display the PBP (fragment) of VIM.

FIG. 15F is a scatterplot showing a densitometric quantitation of intact VIM and the 38 kDa and 26 kDa VIM breakdown products. The mean and SEM are shown. * indicates statistical significance over naive (p-value <0.05, 2 tailed unpaired T-test). This example further shows that biofluid-based monitoring of VIM PBPs or PFs can be used to monitor astrocyte damage.

### Example 15

### Classic MBP Breakdown Products and their Identification in Human CSF

MBP PFs were identified. FIG. 16A is an MS/MS spectrum of the MBP peptide TQDENPVVHF (amino acids 107-116, based on classic human MBP isoform 1; SEQ ID NO:322), charge+2, monoisotopic m/z 593.96 Da. This peptide was released into CSF from human TBI subjects less than or equal to 24 hours after TBI. The spectrum shows the fragment ions, with Identified b+ and y+ type ions in italics and underline, respectively, in Table 21, below.

TABLE 21
MS/MS Data for FIG. 16A.

| | | | SEQ ID | | | |
|---|---|---|---|---|---|---|
| | b⁺ | b²⁺ | NO: 323 | y⁺ | y²⁺ | |
| 1 | 102.11 | 51.56 | T | | | 10 |
| 2 | 230.24 | 115.63 | Q | 1085.16 | 543.08 | 9 |
| 3 | 345.33 | 173.17 | D | 957.03 | 479.02 | 8 |
| 4 | 474.45 | 237.73 | E | 841.94 | 421.48 | 7 |
| 5 | 588.55 | 294.78 | N | 712.83 | 356.92 | 6 |
| 6 | 685.67 | 343.34 | P | 598.72 | 299.87 | 5 |
| 7 | 784.80 | 392.90 | V | 501.61 | 251.31 | 4 |
| 8 | 883.93 | 442.47 | V | 402.47 | 201.74 | 3 |
| 9 | 1021.08 | 511.04 | H | 303.34 | 152.17 | 2 |
| 10 | | | F | 166.20 | 83.60 | 1 |

The full sequence of human MBP isoform 1 (classic MBP, 21 kDa, 197 amino acids, (NP_001020252.1) is:

SEQ ID NO: 324
MASQKRPSQRHGSKYLATASTMDHARHGFLPRHRDTGILDSIGRFF
GGDRGAPKRGSGKVPWLKPGRSPLPSHARSQPGLCNMYKDSHHPAR
TAHYGSLPQKSHGRTQDENPVVHFFKNIVTPRTPPPSQGKGRGLSL
SRFSWGAEGQRPGFGYGGRASDYKSAHKGFKGVDAQGTLSKIFKLG
GRDSRSGSPMARR.

Underlined and bold residues show the areas where PFs originate.

FIG. 16C is a western blot providing the profile of MBP breakdown products in human CSF (8000 Da) released less than or equal to 24 hours after TBI, compared to controls. An anti-MBP (SMI99 Mab) was used to probe the blot. FIG. 16D is a scatterplot showing densitometric quantitation of the 8000 Da MBP fragment with mean and SEM. * indicates statistical significance over naive (p-value <0.05, 2 tailed unpaired T-test).

FIG. 17 is an MS/MS spectrum for a human MBP isoform 2-specific peptide also identified in human TBI CSF, displaying the fragment ions for this peptide. The MBP isoform 2 peptide was HGSKYLATASTMD (aa 11-24; SEQ ID NO:325), charge 2+, monoisotopic m/z 691.55 Da. Identified b- and y-type ions for the MBP peptide are shown in italics and underline from the database search results in Table 22, below. Peptide ions in italics and underline were found in MS/MS spectra.

TABLE 22
Additional Data for FIG. 17.

| | | | SEQ ID | | | |
|---|---|---|---|---|---|---|
| | b⁺ | b²⁺ | NO:326 | y⁺ | y²⁺ | |
| 1 | 138.15 | 69.58 | H | | | 13 |
| 2 | 195.20 | 98.10 | G | 1245.40 | 623.20 | 12 |
| 3 | 282.28 | 141.64 | S | 1188.35 | 594.68 | 11 |
| 4 | 410.45 | 205.73 | K | 1101.27 | 551.14 | 10 |
| 5 | 573.63 | 287.32 | Y | 973.09 | 487.05 | 9 |
| 6 | 686.79 | 343.90 | L | 809.92 | 405.46 | 8 |
| 7 | 757.87 | 379.44 | A | 696.76 | 348.88 | 7 |
| 8 | 858.97 | 429.99 | T | 625.68 | 313.34 | 6 |
| 9 | 930.05 | 465.53 | A | 524.57 | 262.79 | 5 |
| 10 | 1017.13 | 509.07 | S | 453.49 | 227.25 | 4 |
| 11 | 1118.24 | 559.62 | T | 366.42 | 183.71 | 3 |
| 12 | 1249.43 | 625.22 | M | 265.31 | 133.16 | 2 |
| 13 | | | D | 134.11 | 67.56 | 1 |

The location of the peptide within the N-terminal region of human MBP Isoform 3 (197 aa) accession #167P02686-3 is shown in the sequence (underlined and bold):

SEQ ID NO: 327
MASQKRPSQRHGSKYLATASTMDHARHGFLPRHRDTGILDSIGRFF
GGDRGAPKRGSGKVPWLKPGRSPLPSHARSQPGLCNMYKDSHHPAR
TAHYGSLPQKSHGRTQDENPVVHFFKNIVTPRTPPPSQGKGRGLSL
SRFSWGAEGQRPGFGYGGRASDYKSAHKGFKGVDAQGTLSKIFKLG
GRDSRSGSPMARR.

Additional sequences within this MBP isoform include PRHRDTGILDSIGR; SEQ ID NO:328, GRTQDENPVVHFFKNIVTPRTPPPSQGKGRGLSLSRF; SEQ ID NO:329, and HKGFKGVDAQGTLS; SEQ ID NO:330.

FIG. 18 is an MS/MS spectrum of a human Golli-MBP isoform 1 (304 aa)-specific N-terminal region peptide identified in human TBI CSF, peptide HAGKRELNAEKASTNSETNRGESEKKRNLGELSRTT SEQ ID NO:331 (charge 5+, mono m/z=848.59 Da) found in human Golli-MBP ((304 aa) accession #P02686. Table 23, below, shows identified b- and y-type ions for the Golli-MBP peptide shown in italics from the database search results. Peptide ions in italics and underlined were found in MS/MS spectra.

TABLE 23
MS/MS data for FIG. 18.

| | | | | | | SEQ ID | | | | | | |
|---|---|---|---|---|---|---|---|---|---|---|---|---|
| #1 | b⁺ | b²⁺ | b³⁺ | b⁴⁺ | b⁵⁺ | NO:332 | y⁺ | y²⁺ | y³⁺ | y⁴⁺ | y⁵⁺ | #2 |
| 1 | 138.07 | 69.54 | 46.69 | 35.27 | 28.42 | H | | | | | | 36 |
| 2 | 209.10 | 105.06 | 70.37 | 53.03 | 42.63 | A | 4101.88 | 2051.44 | 1367.96 | 1026.22 | 821.18 | 35 |
| 3 | 266.12 | 133.57 | 89.38 | 67.29 | 54.03 | G | 4030.84 | 2015.92 | 1344.28 | 1008.46 | 806.97 | 34 |
| 4 | 394.22 | 197.61 | 132.08 | 99.31 | 79.65 | K | 3973.82 | 1987.41 | 1325.28 | 994.21 | 795.57 | 33 |
| 5 | 550.32 | 275.66 | 184.11 | 138.34 | 110.87 | R | 3845.72 | 1923.36 | 1282.58 | 962.19 | 769.95 | 32 |
| 6 | 679.36 | 340.19 | 227.13 | 170.60 | 136.68 | E | 3689.62 | 1845.31 | 1230.55 | 923.16 | 738.73 | 31 |
| 7 | 792.45 | 396.73 | 264.82 | 198.87 | 159.30 | L | 3560.58 | 1780.79 | 1187.53 | 890.90 | 712.92 | 30 |
| 8 | 906.49 | 453.75 | 302.83 | 227.38 | 182.10 | N | 3447.49 | 1724.25 | 1149.84 | 862.63 | 690.30 | 29 |
| 9 | 977.53 | 489.27 | 326.51 | 245.14 | 196.31 | A | 3333.45 | 1667.23 | 1111.82 | 834.12 | 667.50 | 28 |
| 10 | 1106.57 | 553.79 | 369.53 | 277.40 | 222.12 | E | 3262.41 | 1631.71 | 1088.14 | 816.36 | 653.29 | 27 |
| 11 | 1234.67 | 617.84 | 412.23 | 309.42 | 247.74 | K | 3133.37 | 1567.19 | 1045.13 | 784.10 | 627.48 | 26 |
| 12 | 1305.70 | 653.35 | 435.91 | 327.18 | 261.95 | A | 3005.28 | 1503.14 | 1002.43 | 752.07 | 601.86 | 25 |
| 13 | 1392.73 | 696.87 | 464.92 | 348.94 | 279.35 | S | 2934.24 | 1467.62 | 978.75 | 734.32 | 587.65 | 24 |
| 14 | 1493.78 | 747.39 | 498.60 | 374.20 | 299.56 | T | 2847.21 | 1424.11 | 949.74 | 712.56 | 570.25 | 23 |
| 15 | 1607.82 | 804.42 | 536.61 | 402.71 | 322.37 | N | 2746.16 | 1373.58 | 916.06 | 687.30 | 550.04 | 22 |
| 16 | 1694.86 | 847.93 | 565.62 | 424.47 | 339.78 | S | 2632.12 | 1316.56 | 878.04 | 658.78 | 527.23 | 21 |
| 17 | 1823.90 | 912.45 | 608.64 | 456.73 | 365.59 | E | 2545.08 | 1273.05 | 849.03 | 637.03 | 509.82 | 20 |
| 18 | 2004.91 | 1002.96 | 668.98 | 501.98 | 401.79 | T (p) | 2416.04 | 1208.52 | 806.02 | 604.77 | 484.01 | 19 |
| 19 | 2118.96 | 1059.98 | 706.99 | 530.49 | 424.60 | N | 2235.03 | 1118.02 | 745.68 | 559.51 | 447.81 | 18 |
| 20 | 2275.06 | 1138.03 | 759.02 | 569.52 | 455.82 | R | 2120.98 | 1061.00 | 707.67 | 531.00 | 425.00 | 17 |
| 21 | 2332.08 | 1166.54 | 778.03 | 583.78 | 467.22 | G | 1964.88 | 982.95 | 655.63 | 491.98 | 393.78 | 16 |
| 22 | 2461.12 | 1231.06 | 821.05 | 616.04 | 493.03 | E | 1907.86 | 954.43 | 636.63 | 477.72 | 382.38 | 15 |
| 23 | 2548.15 | 1274.58 | 850.06 | 637.79 | 510.44 | S | 1778.82 | 889.91 | 593.61 | 445.46 | 356.57 | 14 |
| 24 | 2677.20 | 1339.10 | 893.07 | 670.05 | 536.25 | E | 1691.79 | 846.40 | 564.60 | 423.70 | 339.16 | 13 |
| 25 | 2805.29 | 1403.15 | 935.77 | 702.08 | 561.86 | K | 1562.75 | 781.88 | 521.59 | 391.44 | 313.35 | 12 |
| 26 | 2933.39 | 1467.20 | 978.47 | 734.10 | 587.48 | K | 1434.65 | 717.83 | 478.89 | 359.42 | 287.74 | 11 |
| 27 | 3089.49 | 1545.25 | 1030.50 | 773.13 | 618.70 | R | 1306.56 | 653.78 | 436.19 | 327.39 | 262.12 | 10 |
| 28 | 3203.53 | 1602.27 | 1068.51 | 801.64 | 641.51 | N | 1150.45 | 575.73 | 384.16 | 288.37 | 230.90 | 9 |
| 29 | 3316.61 | 1658.81 | 1106.21 | 829.91 | 664.13 | L | 1036.41 | 518.71 | 346.14 | 259.86 | 208.09 | 8 |
| 30 | 3373.64 | 1687.32 | 1125.22 | 844.16 | 675.53 | G | 923.33 | 462.17 | 308.45 | 231.59 | 185.47 | 7 |
| 31 | 3502.68 | 1751.84 | 1168.23 | 876.43 | 701.34 | E | 866.31 | 433.66 | 289.44 | 217.33 | 174.07 | 6 |
| 32 | 3615.76 | 1808.38 | 1205.93 | 904.70 | 723.96 | L | 737.26 | 369.14 | 246.43 | 185.07 | 148.26 | 5 |
| 33 | 3702.79 | 1851.90 | 1234.94 | 926.45 | 741.36 | S | 624.18 | 312.59 | 208.73 | 156.80 | 125.64 | 4 |
| 34 | 3858.90 | 1929.95 | 1286.97 | 965.48 | 772.58 | R | 537.15 | 269.08 | 179.72 | 135.04 | 108.24 | 3 |
| 35 | 4039.91 | 2020.46 | 1347.31 | 1010.73 | 808.79 | T (p) | 381.05 | 191.03 | 127.69 | 96.02 | 77.01 | 2 |
| 36 | | | | | | T (p) | 200.03 | 100.52 | 67.35 | 50.76 | 40.81 | 1 |

The full sequence of human Golli-MBP1, accession #P02686 (304 aa; 34 kDa), is:

SEQ ID NO: 333
MGN HAGKREL NAEKASTNSE TNRGESEKKR NLGELSRTTS
EDNEVFGEAD ANQNNGTSSQ DTAVTDSKRT ADPK NAWQDA
HPADPGSRPH LIRLFSRDAP GREDNTFKDR PSESDELQTI
QEDSAATSES LDV MASQKRP SQR HGSKYLA TASTMDHARH
GFLPRHRDTG ILDSIGRFFG GDRGAPKRGS GKDSHHPART
AHYGSLPQKS H GRTQDENPV VHFFKNIVTP RTPPPSQGKG
RGLSLSRFSW GAEGQRPGFG YGGRASDYKS A HKGFKGVDA
QGTLSKIFKL GGRDSRSGSP MARR.

The italic sequence in Golli-MBP isoform 1 above is identical to that of human MBP isoform 5 (#P02686-5, 171 aa).

Golli-MBP isoform 1 PFs found in human TBI CSF ultrafiltrate samples are of the following sequences: residues 4-34 of this Golli-MBP isoform 1 sequence as HAGKRELNAEKASTNSETNRGESEKKRNLGE (SEQ ID NO:334); residues 75-116 of this sequence as NAWQDAHPADPGSRPHLIRLFSRDAPGREDNTFKDRPSESDE (SEQ ID NO:335). These two peptide unique fragments are derived from the N-terminal region of Golli-MBP isoform 1, and are not found in classical MBP isoform 5. Additional Golli-MBP isoform 1 PFs found in human TBI CSF ultrafiltrate samples are of the following sequences: residues 144-157 of this sequence of Golli-MBP1, accession #P02686 (304 aa) as HGSKYLATASTMDH (SEQ ID NO:336); residues 164-177 as PRHRDTGILDSIGR (SEQ ID NO:337; residues 212-248 as GRTQDENPVVHFFKNIVTPRTPPPSQGKGRGLSLSRF (SEQ ID NO:338); and residues 272-285 as HKGFKGVDAQGTLS (SEQ ID NO:339). These sequences are found in both the Golli-MBP isoform and classical MBP isoform 5. These PF sequences in the Golli-MBP isoform 1 sequence are shown as underlined (see above SEQ ID NO:340aa).

The full sequence of human MBP isoform 5; #P02686-5; 171 aa; 18.5 kDa) is:

(SEQ ID NO: 341bb)
MASQKRPSQRHGSKYLATASTMDHARHGFLPRHRDTGILDSIGRFFGGDR
GAPKRGSGKDSHHPARTAHYGSLPQKSHGRTQDENPVVHFFKNIVTPRTP
PPSQGKGRGLSLSRFSWGAEGQRPGFGYGGRASDYKSAHKGFKGVDAQGT
LSKIFKLGGRDSRSGSPMARR.

Underlined sequences are MBP PFs identified in human TBI CSF ultrafiltrate samples as shown above.

Table 23, below, summarizes MBP PFs found in human TBI CSF that are derived from both human Golli-MBP1 (304 aa, #P02686-1) and MBP Isoform 3 ((171 aa; #P02686-5). The sequences of human Golli-MBP1 (SEQ ID NO:342aa) and classic MBP Isoform 3 (SEQ ID NO:343bb) are shown. The common regions of both isoforms are in italics. PFs derived from a distinct N-terminal region identified in Golli-MBP1 (SEQ ID NO:344aa) are shown in italics. This example further shows that biofluid-based monitoring of classic MBP (e.g., MBP3, MBP5) and Golli-MBP1 fragments or peptides can be used to monitor oligodendrocyte/myelin damage/white matter injury.

Table 23 presents selected PFs detected in human CSF samples from TBI subjects. See Table 24, below.

TABLE 24
Select MBP Biomarker Peptides.

| | | amino acid | SEQ ID |
|---|---|---|---|
| Name | Sequence | residues | NO |
| “Classic MBP isoform 5 | HGSKYLATASTMD | 11-24 | 345 |
| (P02686-5) N-terminal | | | |
| peptide 1” | | | |
| “Classic MBP isoform 5 | HGSKYLATASTMDHAR | 11-43 | 346 |
| (P02686-5) N-terminal | HGFLPRHRDTGILDSIGR | | |
| peptide 2” | | | |
| “Classic MBP isoform 5 | GRTQDENPVVHFFKNIV | 79-115 | 347 |
| (P02686-5) center | TPRTPPPSQGKGRGLSLS | | |
| peptide” | RF | | |
| “Classic MBP isoform 5 | HKGFKGVDAQGTLS | 139-152 | 348 |
| (P02686-5) C-terminal | | | |
| peptide” | | | |
| “Classic MBP (P02686-5) | TQDENPVVHF | 81-90 | 322 |
| C-terminal peptide” | | | |
| “Golli-MBP isoform | HAGKRELNAEKASTNSE | 4-34 | 350 |
| (P02686-1)-specific N- | TNRGESETNRGESEKKR | | |
| terminal peptide” | NLGE | | |
| “Golli-MBP isoform | NAWQDAHPADPGSRPH | 75-116 | 351 |
| (P02686-1)-specific | LIRLFSRDAPGREDNTFK | | |
| center peptide” | DRPSESDE | | |

### Example 16

### Glial Fibrillary Acid Protein Protein Breakdown Products and Peptide Fragments

FIG. 19A is an MS/MS spectrum of GFAP PF (aa 6 to 43) ITSAARRSYVSSGEMMVGGLAPGRRLGPGTRLSLARMP SEQ ID NO:352, found in human CSF ultrafiltrate; FIG. 19B is an MS/MS spectrum of GFAP PF (14-38) YVSSGEMMVGGLAPGRRLGPGTRLS SEQ ID NO:353, found in human CSF ultrafiltrate;

FIG. 19C is an MS/MS spectrum of GFAP PF DGEVIKES SEQ ID NO:354; FIG. 19D is an MS/MS spectrum of GFAP PF DGEVIKE SEQ ID NO:355; FIG. 19E is an MS/MS spectrum of GFAP PF GEENRITIPVQTFSNLQIRETSLDTKSV SEQ ID NO:356. Tables 25, 26, 27, 28, and 29, below, present additional data. Peptide ions in italics and underline were identified in the MS/MS spectra in the four tables.

TABLE 25
MS/MS Data for FIG. 19A.

| | | | | SEQ ID | | | | |
|---|---|---|---|---|---|---|---|---|
| #1 | b⁺ | b²⁺ | b³⁺ | NO:357 | y⁺ | y²⁺ | y³⁺ | #2 |
| 1 | 114.09134 | 57.54931 | 38.70196 | I | | | | 38 |
| 2 | 215.13902 | 108.07315 | 72.38452 | T | 4101.90990 | 2051.45859 | 1367.97482 | 37 |
| 3 | 302.17105 | 151.58916 | 101.39520 | S | 4000.86222 | 2000.93475 | 1334.29226 | 36 |
| 4 | 373.20816 | 187.10772 | 125.07424 | A | 3913.83019 | 1957.41874 | 1305.28158 | 35 |
| 5 | 444.24527 | 222.62628 | 148.75328 | A | 3842.79308 | 1921.90018 | 1281.60254 | 34 |
| 6 | 600.34639 | 300.67683 | 200.78698 | R | 3771.75597 | 1886.38162 | 1257.92351 | 33 |
| 7 | 756.44750 | 378.72739 | 252.82068 | R | 3615.65486 | 1808.33107 | 1205.88980 | 32 |
| 8 | 843.47953 | 422.24340 | 281.83136 | S | 3459.55374 | 1730.28051 | 1153.85610 | 31 |
| 9 | 1006.54285 | 503.77507 | 336.18580 | Y | 3372.52172 | 1686.76450 | 1124.84542 | 30 |
| 10 | 1105.61127 | 553.30927 | 369.20861 | V | 3209.45839 | 1605.23283 | 1070.49098 | 29 |
| 11 | 1272.60963 | 636.80845 | 424.87473 | S (p) | 3110.38997 | 1555.69863 | 1037.46818 | 28 |
| 12 | 1439.60799 | 720.30763 | 480.54085 | S (p) | 2943.39161 | 1472.19945 | 981.80206 | 27 |
| 13 | 1496.62945 | 748.81836 | 499.54800 | G | 2776.39326 | 1388.70027 | 926.13594 | 26 |
| 14 | 1625.67204 | 813.33966 | 542.56220 | E | 2719.37179 | 1360.18953 | 907.12878 | 25 |
| 15 | 1772.70744 | 886.85736 | 591.57400 | M (ox) | 2590.32920 | 1295.66824 | 864.11458 | 24 |
| 16 | 1903.74793 | 952.37760 | 635.25416 | M | 2443.29380 | 1222.15054 | 815.10278 | 23 |
| 17 | 2002.81634 | 1001.91181 | 668.27696 | V | 2312.25331 | 1156.63030 | 771.42262 | 22 |
| 18 | 2059.83780 | 1030.42254 | 687.28412 | G | 2213.18490 | 1107.09609 | 738.39982 | 21 |
| 19 | 2116.85927 | 1058.93327 | 706.29127 | G | 2156.16344 | 1078.58536 | 719.39266 | 20 |
| 20 | 2229.94333 | 1115.47530 | 743.98596 | L | 2099.14197 | 1050.07462 | 700.38551 | 19 |
| 21 | 2300.98045 | 1150.99386 | 767.66500 | A | 1986.05791 | 993.53259 | 662.69082 | 18 |
| 22 | 2398.03321 | 1199.52024 | 800.01592 | P | 1915.02080 | 958.01404 | 639.01178 | 17 |
| 23 | 2455.05467 | 1228.03098 | 819.02308 | G | 1817.96803 | 909.48765 | 606.66086 | 16 |
| 24 | 2611.15578 | 1306.08153 | 871.05678 | R | 1760.94657 | 880.97692 | 587.65371 | 15 |
| 25 | 2767.25690 | 1384.13209 | 923.09048 | R | 1604.84546 | 802.92637 | 535.62000 | 14 |
| 26 | 2880.34096 | 1440.67412 | 960.78517 | L | 1448.74435 | 724.87581 | 483.58630 | 13 |
| 27 | 2937.36242 | 1469.18485 | 979.79233 | G | 1335.66028 | 668.33378 | 445.89161 | 12 |
| 28 | 3034.41519 | 1517.71123 | 1012.14325 | P | 1278.63882 | 639.82305 | 426.88446 | 11 |
| 29 | 3091.43665 | 1546.22196 | 1031.15040 | G | 1181.58605 | 591.29667 | 394.53354 | 10 |
| 30 | 3192.48433 | 1596.74580 | 1064.83296 | T | 1124.56459 | 562.78593 | 375.52638 | 9 |
| 31 | 3348.58544 | 1674.79636 | 1116.86666 | R | 1023.51691 | 512.26209 | 341.84382 | 8 |
| 32 | 3461.66950 | 1731.33839 | 1154.56135 | L | 867.41580 | 434.21154 | 289.81012 | 7 |
| 33 | 3628.66786 | 1814.83757 | 1210.22747 | S (p) | 754.33174 | 377.66951 | 252.11543 | 6 |
| 34 | 3741.75193 | 1871.37960 | 1247.92216 | L | 587.33338 | 294.17033 | 196.44931 | 5 |
| 35 | 3812.78904 | 1906.89816 | 1271.60120 | A | 474.24931 | 237.62830 | 158.75462 | 4 |
| 36 | 3968.89015 | 1984.94871 | 1323.63490 | R | 403.21220 | 202.10974 | 135.07558 | 3 |
| 37 | 4099.93064 | 2050.46896 | 1367.31506 | M | 247.11109 | 124.05918 | 83.04188 | 2 |
| 38 | | | | P | 116.07061 | 58.53894 | 39.36172 | 1 |

TABLE 26
MS/MS Data for FIG. 19B.

| | | | | | SEQ ID | | | | | |
|---|---|---|---|---|---|---|---|---|---|---|
| #1 | b⁺ | b²⁺ | b³⁺ | b⁴⁺ | NO:358 | y⁺ | y²⁺ | y³⁺ | y⁴⁺ | #2 |
| 1 | 164.07061 | 82.53894 | 55.36172 | 41.77311 | Y | | | | | 25 |
| 2 | 263.13902 | 132.07315 | 88.38452 | 66.54021 | V | 2481.21019 | 1241.10873 | 827.74158 | 621.05800 | 24 |
| 3 | 430.13738 | 215.57233 | 144.05064 | 108.28980 | S (p) | 2382.14177 | 1191.57453 | 794.71878 | 596.29090 | 23 |
| 4 | 517.16941 | 259.08834 | 173.06132 | 130.04781 | S | 2215.14342 | 1108.07535 | 739.05266 | 554.54131 | 22 |
| 5 | 574.19087 | 287.59907 | 192.06847 | 144.30318 | G | 2128.11139 | 1064.55933 | 710.04198 | 532.78330 | 21 |
| 6 | 703.23346 | 352.12037 | 235.08267 | 176.56382 | E | 2071.08992 | 1036.04860 | 691.03483 | 518.52794 | 20 |
| 7 | 850.26886 | 425.63807 | 284.09447 | 213.32267 | M (ox) | 1942.04733 | 971.52730 | 648.02063 | 486.26729 | 19 |
| 8 | 981.30935 | 491.15831 | 327.77463 | 246.08279 | M | 1795.01193 | 898.00960 | 599.00883 | 449.50844 | 18 |
| 9 | 1080.37776 | 540.69252 | 360.79744 | 270.84990 | V | 1663.97145 | 832.48936 | 555.32867 | 416.74832 | 17 |
| 10 | 1137.39923 | 569.20325 | 379.80459 | 285.10526 | G | 1564.90303 | 782.95515 | 522.30586 | 391.98122 | 16 |
| 11 | 1194.42069 | 597.71398 | 398.81175 | 299.36063 | G | 1507.88157 | 754.44442 | 503.29871 | 377.72585 | 15 |
| 12 | 1307.50475 | 654.25601 | 436.50644 | 327.63165 | L | 1450.86011 | 725.93369 | 484.29155 | 363.47048 | 14 |
| 13 | 1378.54187 | 689.77457 | 460.18547 | 345.39092 | A | 1337.77604 | 669.39166 | 446.59686 | 335.19947 | 13 |
| 14 | 1475.59463 | 738.30095 | 492.53639 | 369.65412 | P | 1266.73893 | 633.87310 | 422.91783 | 317.44019 | 12 |
| 15 | 1532.61609 | 766.81169 | 511.54355 | 383.90948 | G | 1169.68616 | 585.34672 | 390.56691 | 293.17700 | 11 |
| 16 | 1688.71720 | 844.86224 | 563.57725 | 422.93476 | R | 1112.66470 | 556.83599 | 371.55975 | 278.92163 | 10 |
| 17 | 1844.81832 | 922.91280 | 615.61096 | 461.96004 | R | 956.56359 | 478.78543 | 319.52605 | 239.89635 | 9 |
| 18 | 1957.90238 | 979.45483 | 653.30564 | 490.23105 | L | 800.46248 | 400.73488 | 267.49234 | 200.87108 | 8 |
| 19 | 2014.92384 | 1007.96556 | 672.31280 | 504.48642 | G | 687.37841 | 344.19285 | 229.79766 | 172.60006 | 7 |
| 20 | 2111.97661 | 1056.49194 | 704.66372 | 528.74961 | P | 630.35695 | 315.68211 | 210.79050 | 158.34470 | 6 |
| 21 | 2168.99807 | 1085.00267 | 723.67087 | 543.00498 | G | 533.30419 | 267.15573 | 178.43958 | 134.08150 | 5 |
| 22 | 2270.04575 | 1135.52651 | 757.35343 | 568.26689 | T | 476.28272 | 238.64500 | 159.43243 | 119.82614 | 4 |
| 23 | 2426.14686 | 1213.57707 | 809.38714 | 607.29217 | R | 375.23504 | 188.12116 | 125.74987 | 94.56422 | 3 |
| 24 | 2539.23092 | 1270.11910 | 847.08183 | 635.56319 | L | 219.13393 | 110.07061 | 73.71616 | 55.53894 | 2 |
| 25 | | | | | S | 106.04987 | 53.52857 | 36.02147 | 27.26792 | 1 |

TABLE 27
MS/MS Data for FIG. 19C.

| | | | SEQ ID | | | |
|---|---|---|---|---|---|---|
| #1 | b⁺ | b²⁺ | NO:359 | y⁺ | y²⁺ | #2 |
| 1 | 116.03422 | 58.52075 | D | | | 8 |
| 2 | 173.05568 | 87.03148 | G | 761.40396 | 381.20562 | 7 |
| 3 | 302.09828 | 151.55278 | E | 704.38250 | 352.69489 | 6 |
| 4 | 401.16669 | 201.08698 | V | 575.33990 | 288.17359 | 5 |
| 5 | 514.25075 | 257.62902 | I | 476.27149 | 238.63938 | 4 |
| 6 | 642.34572 | 321.67650 | K | 363.18743 | 182.09735 | 3 |
| 7 | 771.38831 | 386.19779 | E | 235.09246 | 118.04987 | 2 |
| 8 | | | S | 106.04987 | 53.52857 | 1 |

TABLE 28
MS/MS Data for Table 19D.

| | | | SEQ ID | | | |
|---|---|---|---|---|---|---|
| #1 | b⁺ | b²⁺ | NO: 360 | y⁺ | y²⁺ | #2 |
| 1 | 116.03422 | 58.52075 | D | | | 7 |
| 2 | 173.05568 | 87.03148 | G | 674.37193 | 337.68960 | 6 |
| 3 | 302.09828 | 151.55278 | E | 617.35047 | 309.17887 | 5 |
| 4 | 401.16669 | 201.08698 | V | 488.30788 | 244.65758 | 4 |
| 5 | 514.25075 | 257.62902 | I | 389.23946 | 195.12337 | 3 |
| 6 | 642.34572 | 321.67650 | K | 276.15540 | 138.58134 | 2 |
| 7 | | | E | 148.06043 | 74.53386 | 1 |

TABLE 29
MS/MS Data for Table 19E.

| | | | | | SEQ ID | | | | | |
|---|---|---|---|---|---|---|---|---|---|---|
| #1 | b⁺ | b²⁺ | b³⁺ | b⁴⁺ | NO: 361 | y⁺ | y²⁺ | Y³⁺ | y⁴⁺ | #2 |
| 1 | 58.02874 | 29.51801 | 20.01443 | 15.26264 | G | | | | | 28 |
| 2 | 187.07133 | 94.03930 | 63.02863 | 47.52329 | E | 3118.63788 | 1559.82258 | 1040.21748 | 780.41493 | 27 |
| 3 | 316.11393 | 158.56060 | 106.04283 | 79.78394 | E | 2989.59529 | 1495.30128 | 997.20328 | 748.15428 | 26 |
| 4 | 430.15685 | 215.58207 | 144.05714 | 108.29467 | N | 2860.55270 | 1430.77999 | 954.18908 | 715.89363 | 25 |
| 5 | 586.25796 | 293.63262 | 196.09084 | 147.31995 | R | 2746.50977 | 1373.75852 | 916.17477 | 687.38290 | 24 |
| 6 | 699.34203 | 350.17465 | 233.78553 | 175.59096 | I | 2590.40866 | 1295.70797 | 864.14107 | 648.35762 | 23 |
| 7 | 800.38971 | 400.69849 | 267.46809 | 200.85288 | T | 2477.32459 | 1239.16593 | 826.44638 | 620.08661 | 22 |
| 8 | 913.47377 | 457.24052 | 305.16277 | 229.12390 | I | 2376.27691 | 1188.64210 | 792.76382 | 594.82469 | 21 |
| 9 | 1010.52653 | 505.76691 | 337.51370 | 253.38709 | P | 2263.19285 | 1132.10006 | 755.06913 | 566.55367 | 20 |
| 10 | 1109.59495 | 555.30111 | 370.53650 | 278.15419 | V | 2166.14009 | 1083.57368 | 722.71821 | 542.29048 | 19 |
| 11 | 1237.65353 | 619.33040 | 413.22269 | 310.16884 | Q | 2067.07167 | 1034.03947 | 689.69541 | 517.52338 | 18 |
| 12 | 1338.70120 | 669.85424 | 446.90525 | 335.43076 | T | 1939.01310 | 970.01019 | 647.00922 | 485.50873 | 17 |
| 13 | 1485.76962 | 743.38845 | 495.92806 | 372.19786 | F | 1837.96542 | 919.48635 | 613.32666 | 460.24681 | 16 |
| 14 | 1572.80165 | 786.90446 | 524.93873 | 393.95587 | S | 1690.89700 | 845.95214 | 564.30385 | 423.47971 | 15 |
| 15 | 1686.84457 | 843.92593 | 562.95304 | 422.46660 | N | 1603.86498 | 802.43613 | 535.29318 | 401.72170 | 14 |
| 16 | 1799.92864 | 900.46796 | 600.64773 | 450.73762 | L | 1489.82205 | 745.41466 | 497.27887 | 373.21097 | 13 |
| 17 | 1927.98722 | 964.49725 | 643.33392 | 482.75226 | Q | 1376.73798 | 688.87263 | 459.58418 | 344.93995 | 12 |
| 18 | 2041.07128 | 1021.03928 | 681.02861 | 511.02328 | I | 1248.67941 | 624.84334 | 416.89799 | 312.92531 | 11 |
| 19 | 2197.17239 | 1099.08983 | 733.06231 | 550.04856 | R | 1135.59534 | 568.30131 | 379.20330 | 284.65429 | 10 |
| 20 | 2326.21498 | 1163.61113 | 776.07651 | 582.30920 | E | 979.49423 | 490.25075 | 327.16959 | 245.62902 | 9 |
| 21 | 2427.26266 | 1214.13497 | 809.75907 | 607.57112 | T | 850.45164 | 425.72946 | 284.15540 | 213.36837 | 8 |
| 22 | 2514.29469 | 1257.65098 | 838.76975 | 629.32913 | S | 749.40396 | 375.20562 | 250.47284 | 188.10645 | 7 |
| 23 | 2627.37875 | 1314.19302 | 876.46444 | 657.60015 | L | 662.37193 | 331.68960 | 221.46216 | 166.34844 | 6 |
| 24 | 2742.40570 | 1371.70649 | 914.80675 | 686.35688 | D | 549.28787 | 275.14757 | 183.76747 | 138.07742 | 5 |
| 25 | 2843.45338 | 1422.23033 | 948.48931 | 711.61880 | T | 434.26092 | 217.63410 | 145.42516 | 109.32069 | 4 |
| 26 | 2971.54834 | 1486.27781 | 991.18763 | 743.64254 | K | 333.21325 | 167.11026 | 111.74260 | 84.05877 | 3 |
| 27 | 3058.58037 | 1529.79382 | 1020.19831 | 765.40055 | S | 205.11828 | 103.06278 | 69.04428 | 52.03503 | 2 |
| 28 | | | | | V | 118.08626 | 59.54677 | 40.03360 | 30.27702 | 1 |

Overall, these data indicate that a number of GFAP PFs in the GFAP alpha isoform (#P14136; 432 aa) are found in TBI CSF samples and can serve as biomarkers for TBI or traumatic injury to the CNS. This example also shows that human biofluid-based monitoring of PFs of GFAP can be used to monitor astroglia injury. See Table 30, below for a list of selected peptides.

The full sequence of glial fibrillary acidic protein (human) alpha isoform (#P14136; GI:251802 (with the regions where the PFs occur shown in bold) is:

SEQ ID NO: 362
MERRRITSAARRSYVSSGEMMVGGLAPGRRLGPGTRLSLARMPPPLPTRV
DFSLAGALNAGFKETRASERAEMMELNDRFASYIEKVRFLEQQNKALAAE
LNQLRAKEPTKLADVYQAELRELRLRLDQLTANSARLEVERDNLAQDLAT
VRQKLQDETNLRLEAENNLAAYRQEADEATLARLDLERKIESLEEEIRFL
RKIHEEEVRELQEQLARQQVHVELDVAKPDLTAALKEIRTQYEAMASSNM
HEAEEWYRSKFADLTDAAARNAELLRQAKHEANDYRRQLQSLTCDLESLR
GTNESLERQMREQEERHVREAASYQEALALEEEGQSLKDEMARHLQEYQD
LLNVKLALDIEIATYRKLLEGEENRITIPVQTFSNLQIRETSLDTKSVSE
GHLKRNIVVKTVEMRDGEVIKESKQEHKDVM.

TABLE 30
Select GFAP Peptide Breakdown Products.

| | | amino acid | SEQ ID |
|---|---|---|---|
| Name | Sequence | residues | NO |
| “GFAP N-terminal | ITSAARRSYVSSGEMMVGGLAPGR | 6-43 | 363 |
| peptide 1” | RLGPGTRLSLARMP | | |
| “GFAP N-terminal | YVSSGEMMVGGLAPGRRLGPGTRLS | 14-38 | 364 |
| peptide 2” | | | |
| “GFAP N-terminal | RSYVSSGEMMVGGLAPGRRLGP | 12-33 | 365 |
| peptide 3” | | | |
| “GFAP N-terminal | AARRSYVSSGEMMVGGLAPGRRL | 9-49 | 366 |
| peptide 4” | GPGTRLSLARMPPPLPTR | | |
| “GFAP C-terminal | GEENRITIPVQTFSNLQIRETSLDTK | 372-399 | 367 |
| peptide 1” | SV | | |
| “GFAP C-terminal | QTFSNLQIRETSLDTKSVSEGHLKR | 382-416 | 368 |
| peptide 2” | NIVVKTVEMR | | |
| “GFAP C-terminal | DGEVIKES | 417-423 | 369 |
| peptide 3” | | | |
| “GFAP C-terminal | DGEVIKE | 417-422 | 370 |
| peptide 4” | | | |
| “GFAP C-terminal | DGEVIKESKQEHKDVM | 417-332 | 371 |
| peptide 5” | | | |

### Example 17

### Tau Protein Breakdown Products and Peptide Fragments from Human TBI CSF Ultrafiltrate Samples

FIG. 20A shows that an Isoform Tau441 (Tau4/Tau-441; identifier: P10636-8; 441aa) PF AEPRQEFEVMEDHAGTYGLGDRKDQGGYT (aa 2-30; SEQ ID NO:372) is found in the ultrafiltrate of human TBI CSF samples. See also Table 31, below. All sequences are of High Confidence.

FIG. 20B shows Tau-441 (P10636-8, 441 aa)C-terminal peptide [419-441] VDSPQLATLADEVSASLAK is among of the most significantly elevated PF detected in human TBI CSF samples (versus control CSF) using high resolution tandem mass spectrometry, as supported by a plot of Log Student's T-test p value Day 2 TBI versus control. Vs. Student's T-test Difference Day 2 vs. control. This peptide is found in both Tau-441 (Tau-F) and Tau-G isoforms.

FIG. 20C show a compilation of additional Tau-441 (P10636-8, 441 aa)N-terminal peptide [2-30]AEPRQEFEVMEDHAGTYGLGDRKDQGGYT (SEQ ID NO: 373, and C-terminal peptide [421-438] SPQLATLADEVSASLAK (SEQ ID NO: 474). Additional peptides found in TBI are shown. Duplicate peptides found are not shown. Sequence numbers are shown on the y-axis and are based on human tau-441. None of the peptides shown were found in control CSF samples.

TABLE 31
Additional Data for FIG. 20 (P10636-8 peptides).

| | | | | | | XCorr by |
|---|---|---|---|---|---|---|
| | Position | | | No. | | search |
| | in | | Protein | of | Theo. | engine: A8 |
| Sequence | Protein | Modification | Modification | PSM | MH+ (Da) | Sequest HT |
| AEPRQEFEVMEDHAGTYG | 2-30 | 1xOxidation | 3xPhospho | 1 | 3512.3539 | 2.19 |
| LGDRKDQGGYT | | [M10]; 3xPhospho | [Y29(99.6); | | | |
| (SEQ ID NO: 373) | | [Y28(99.6); | T30(99.6); Y/T] | | | |
| | | T29(99.6); Y/T] | | | | |
| KTPPSSG | 180-186 | 3xPhospho | 3xPhospho | 1 | 913.25052 | 1.32 |
| (SEQ ID NO: 374) | | [T2(100); S5(100); | [T181(100); | | | |
| | | S6(100)] | S184(100); | | | |
| | | | S185(100)] | | | |
| PGSRSRTPSLPTPP | 206-219 | 3xPhospho | 3xPhospho | 1 | 1689.67984 | 1.36 |
| (SEQ ID NO: 375) | | [S9(98.6); | [S214(98.6); | | | |
| | | T12(99.9); T/S] | T217(99.9); | | | |
| | | | S/T] | | | |
| PVVSGDTSPRHLSNVSS | 397-413 | 3xPhospho | 3xPhospho | 1 | 1978.77084 | 2.15 |
| (SEQ ID NO: 376) | | [S4(77.2); S/T] | [S400(77.2); | | | |
| | | | S/T] | | | |
| VKSEKLDFKDRVQSKIGSL | 339-357 | 3xPhospho | 3xPhospho | 1 | 2417.12784 | 2.29 |
| (SEQ ID NO: 377) | | [S3(100); | [S341(100); | | | |
| | | S14(100); | S352(100); | | | |
| | | S18(100)] | S356(100) | | | |
| YKPVDLSKVTSKCGSL | 310-325 | 1xOxidation | 1xOxidation | 1 | 1980.81904 | 2.34 |
| (SEQ ID NO: 378) | | [C13]; 3xPhospho | [C322]; | | | |
| | | [Y1(97.7); | 3xPhospho | | | |
| | | S15(95.8); T/S] | [Y310(97.7); | | | |
| | | | S324(95.8); T/S] | | | |

Table 32, below provides a list of PFs showing an isoform specific peptide for the high molecular weight Tau-758 (identifier: P10636-19; 776aa). These PFs can be detected in TBI CSF samples, but in not control CSF.

TABLE 32
Tau-758 and Tau-441 Peptide Fragments Found in Human TBI CSF
Ultrafiltrate Samples.

| | | |
|---|---|---|
| | Position in | SEQ ID |
| Sequence | Protein | NO |

TAU-758 (P10636-9)

| | | |
|---|---|---|
| PQLKARMVSKSKDGTGSDDKKAKTSTRSSA | 372-401 | 379 |
| PPSSPKYVSSVTSRTGSSGAKEMKLKGADGKTKIATPRGAA | 435-475 | 380 |
| LKARMVSKSKDGTGSDDKKAKTSTRSSAKTLKNRPCLSPKHPTPGS | 374-419 | 381 |
| SVTSRTGSSGAKEMKLKGADGK | 444-465 | 382 |
| EDRDVDESSPQDSPPSKASPAQDGRPPQTAAREATSIPGFPAEGAIP | 220-266 | 383 |
| SPKHPTPGSSDPLIQPSSPAVCPEPPSSPKYVSSVTSRTGSSGAKEM | 411-457 | 384 |
| PQLKARMVSKSKDGTGSDDKKAKTSTRSSA | 372-401 | 385 |
| PPSSPKYVSSVTSRTGSSGAKEMKLKGADGKTKIATPRGAA | 435-486 | 387 |
| SVTSRTGSSGAKEMKLKGADGK | 444-465 | 388 |

TAU-441 (P10636-8)

| | | |
|---|---|---|
| SPQLATLADEVSASLAK | 421-438 | 474 |
| EIVYKSPVVSGDTSPRHLSNVSSTGSIDMVDSPQLATLADEVSA | 391-434 | 389 |
| KHVPGGGSVQIVYKPVDLS | 298-316 | 390 |
| KNVKSKIGSTENL | 254-266 | 391 |
| PLQTPTEDGSEEPGSETSDAKSTPTAEDVTAPLVDEGAPGKQAAAQPHT | 47-95 | 392 |
| SKCGSKD | 289-295 | 393 |

The following tables show the sequences of Tau-441 and Tau-758. The isoform unique sequences are underlined. The Tau-758 PFs found in human TBI CSF ultrafiltrate are shown in bold. See Table 33 and Table 34, below.

TABLE 33
Human Isoform Tau-F (Tau-441) of Microtubule-
associated protein tau (identifier: P10636-8).

MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQARMVSKSKDGTGSDDKKAKGADGKTK
IATPRGAAPPGQKGQANATRIPAKTPPAPKTPPSSGEPPKSGDRSGYSSP
GSPGTPGSRSRTPSLPTPPTREPKKVAVVRTPPKSPSSAKSRLQTAPVPM
PDLKNVKSKIGSTENLKHQPGGGKVQIINKKLDLSNVQSKCGSKDNIKHV
PGGGSVQIVYKPVDLSKVTSKCGSLGNIHHKPGGGQVEVKSEKLDFKDRV
QSKIGSLDNITHVPGGGNKKIETHKLTFRENAKAKTDHGAEIVYKSPVVS
GDTSPRHLSNVSSTGSIDMVDSPQLATLADEVSASLAKQGL. SEQ ID
NO: 394
N- and C-terminal regions PFs are originated are shown in bold and underlined. Other PF regions in the central are shown in bold.

TABLE 34
Human Isoform Tau-G (Tau-758) of Microtubule-
associated protein tau (identifier: P10636-1).

MAEPRQEFEVMEDHAGTYGLGDRKDQGGYTMHQDQEGDTDAGLKESPLQT
PTEDGSEEPGSETSDAKSTPTAEDVTAPLVDEGAPGKQAAAQPHTEIPEG
TTAEEAGIGDTPSLEDEAAGHVTQ[EPESGKVVQEGFLREPGPPGLSHQL
MSGMPGAPLLPEGPREATRQPSGTGPEDTEGGRHAPELLKHQLLGDLHQE
GPPLKGAGGKERPGSKEEVDEDRDVDESSPQDSPPSKASPAQDGRPPQTA
AREATSIPGFPAEGAIPLPVDFLSKVSTEIPASEPDGPSVGRAKGQDAPL
EFTFHVEITPNVQKEQAHSEEHLGRAAFPGAPGEGPEARGPSLGEDTKEA
DLPEPSEKQPAAAPRGKPVSRVPQLKARMVSKSKDGTGSDDKKAKTSTRS
SAKTLKNRPCLSPKHPTPGSSDPLIQPSSPAVCPEPPSSPKYVSSVTSRT
GSSGAKEMKL]KGADGKTKIATPRGAAPPGQKGQANATRIPAKTPPAPKT
PPSSATKQVQRRPPPAGPRSERGEPPKSGDRSGYSSPGSPGTPGSRSRTP
SLPTPPTREPKKVAVVRTPPKSPSSAKSRLQTAPVPMPDLKNVKSKIGST
ENLKHQPGGGKVQIINKKLDLSNVQSKCGSKDNIKHVPGGGSVQIVYKPV
DLSKVTSKCGSLGNIHHKPGGGQVEVKSEKLDFKDRVQSKIGSLDNITHV
PGGGNKKIETHKLTFRENAKAKTDHGAEIVYKSPVVSGDTSPRHLSNVSS
TGSIDMVDSPQLATLADEVSASLAKQGL. SEQ ID NO: 395
Central region that is unique to Tau-G, not present in Tau-F form are in square brackets. Central regions PFs are originated are shown in bold and underlined.

Table 35, below, provides a summary of MS/MS results on PFs identified from Tau protein isoforms Tau-758 and Tau-441 in human TBI CSF ultrafiltrate samples.

TABLE 35
Peptide Fragments Identified from Tau Protein Isoforms.

| | | Peptide | | SEQ ID |
|---|---|---|---|---|
| Sequence | Tau Isoforms | Position | Modifications | NO |
| PQLKARMVSKSKDGTGSDDKKAKTS | Tau-776, Tau-758 | 372-401 | 1xOxidation [M7]; | 396 |
| TRSSA | | 372-401 | 2xPhospho [T/S] | |
| PPSSPKYVSSVTSRTGSSGAKEMKLK | Tau-776, Tau-758 | 435-475 | 1xPhospho [S/Y/T] | 397 |
| GADGKTKIATPRGAA | | 435-475 | | |
| LKARMVSKSKDGTGSDDKKAKTSTR | Tau-776, Tau-758 | 374-419 | 1xOxidation [M5]; | 398 |
| SSAKTLKNRPCLSPKHPTPGS | | 379-419 | 1xPhospho [T/S] | |
| SVTSRTGSSGAKEMKLKGADGK | Tau-776, Tau-758 | 444-465 | 1xPhospho [S/T] | 399 |
| | | 444-465 | | |
| EDRDVDESSPQDSPPSKASPAQDGRPP | Tau-776, Tau-758 | 220-266 | 2xPhospho | 400 |
| QTAAREATSIPGFPAEGAIP | | 220-266 | [S19(84); S/T] | |
| SPKHPTPGSSDPLIQPSSPAVCPEPPSSP | Tau-776 Tau-758 | 411-457 | 3xPhospho [S34; | 401 |
| KYVSSVTSRTGSSGAKEM | | 411-457 | S37; S41] | |
| EIVYKSPVVSGDTSPRHLSNVSSTGSI | Tau-441, | 726-769 | 2xPhospho [Y/S/T] | 402 |
| DMVDSPQLATLADEVSA | Tau-776, Tau-758 | 391-434 | | |
| | | 708-751 | | |
| KHVPGGGSVQIVYKPVDLS | Tau-441, | 633-651 | 3xPhospho | 403 |
| | Tau-776, Tau-758 | 298-316 | [S8(100); Y13(100); | |
| | | 615-633 | S19(100)] | |
| KNVKSKIGSTENL | Tau-441, | 589-601 | 1xPhospho [T/S] | 404 |
| | Tau-776, Tau-758 | 254-266 | | |
| | | 571-583 | | |
| PLQTPTEDGSEEPGSETSDAKSTPTAE | Tau-441 | 47-95 | | 405 |
| DVTAPLVDEGAPGKQAAAQPHT | Tau-776 Tau-758 | 47-95 | | |
| | | 47-95 | | |

This example shows that human biofluid-based monitoring of Tau-F (Tau-441) and Tau-G (766 aa) and its PBPs or PFs can be used to monitor axonal injury or neurodegeneration.

### Example 18

### CAMSAP1 Protein Breakdown Products

FIG. 21 is an MS/MS spectrum for the CAMSAP1 peptide SQHGKDPASLLASELVQLH (SEQ ID NO:406) identified in human TBI CSF ultrafiltrate, showing the fragment ions for this peptide. The identified b- and y-type ions for this peptide shown from the database search are provided in Table 36, below. The presence of the CAMSAP1 PF indicates that CAMPSAP1 protein and it high molecular weight fragment/PBP are likely to be released in biofluids such as CSF.

TABLE 36
MS/MS Data for FIG. 21.

| | | | | SEQ ID | | | | |
|---|---|---|---|---|---|---|---|---|
| #1 | b⁺ | b²⁺ | b³⁺ | NO: 407 | Y⁺ | y²⁺ | Y³⁺ | #2 |
| 1 | 88.08539 | 44.54639 | 30.03339 | S | | | | 19 |
| 2 | 216.21630 | 108.61185 | 72.74370 | Q | 1944.19980 | 972.60360 | 648.73819 | 18 |
| 3 | 353.35770 | 177.18255 | 118.45750 | H | 1816.06889 | 908.53814 | 606.02789 | 17 |
| 4 | 410.40940 | 205.70840 | 137.47473 | G | 1678.92749 | 839.96744 | 560.31409 | 16 |
| 5 | 538.58390 | 269.79565 | 180.19956 | K | 1621.87579 | 811.44159 | 541.29686 | 15 |
| 6 | 653.67240 | 327.33990 | 218.56240 | D | 1493.70129 | 747.35434 | 498.57202 | 14 |
| 7 | 750.78920 | 375.89830 | 250.93466 | P | 1378.61279 | 689.81009 | 460.20919 | 13 |
| 8 | 821.86790 | 411.43765 | 274.62756 | A | 1281.49599 | 641.25169 | 427.83692 | 12 |
| 9 | 908.94590 | 454.97665 | 303.65356 | S | 1210.41729 | 605.71234 | 404.14402 | 11 |
| 10 | 1022.10570 | 511.55655 | 341.37350 | L | 1123.33929 | 562.17334 | 375.11802 | 10 |
| 11 | 1135.26550 | 568.13645 | 379.09343 | L | 1010.17949 | 505.59344 | 337.39809 | 9 |
| 12 | 1206.34420 | 603.67580 | 402.78633 | A | 897.01969 | 449.01354 | 299.67816 | 8 |
| 13 | 1293.42220 | 647.21480 | 431.81233 | S | 825.94099 | 413.47419 | 275.98526 | 7 |
| 14 | 1422.53780 | 711.77260 | 474.85086 | E | 738.86299 | 369.93519 | 246.95926 | 6 |
| 15 | 1535.69760 | 768.35250 | 512.57080 | L | 609.74739 | 305.37739 | 203.92072 | 5 |
| 16 | 1634.83040 | 817.91890 | 545.61506 | V | 496.58759 | 248.79749 | 166.20079 | 4 |
| 17 | 1762.96131 | 881.98435 | 588.32537 | Q | 397.45479 | 199.23109 | 133.15652 | 3 |
| 18 | 1876.12111 | 938.56425 | 626.04530 | L | 269.32388 | 135.16564 | 90.44622 | 2 |
| 19 | | | | H | 156.16408 | 78.58574 | 52.72629 | 1 |

FIG. 22A is an immunoblot showing the presence of CAMSAP1 (177 kDa) and its 110 kDa breakdown product in human TBI CSF samples. Both the intact protein and the PBP are present at higher levels in TBI subject CSF than in control CSF (loading 10 uL 3× concentrated CSF). FIG. 22B shows scatterplot data (bars are mean +SEM). CAMSAP1 and CAMSAP-PBP both are higher in TBI CSF than in control CSF (p<0.05, unpaired T-test).

In addition, FIG. 23 is an MS/MS spectrum for the Calmodulin regulated spectrin-associated protein 3 (CAMSAP3) peptide LQEKTEQEAAQ (SEQ ID NO:408) identified in human TBI CSF ultrafiltrate, displaying the fragment ions for this peptide. See Table 37, below for the identified b- and y-type ions for the peptide (indicated) were from the database search. Peptide ions in italics and underline were found in MS/MS spectra.

The presence of proteolytic breakdown products of CAMSAP3 in TBI CSF implies that CAMSAP1 protein and its higher molecular weight breakdown products are present and in higher in biofluids (CSF) from TBI subjects than in controls. This example therefore shows that human biofluid-based monitoring of CAMSAP1 and CAMSAP3 PBPs or PFs can be used to monitor axonal damage.

TABLE 37
MS/MS Data for FIG. 23.

| | | | SEQ ID | | | |
|---|---|---|---|---|---|---|
| #1 | b⁺ | b²⁺ | NO: 409 | Y⁺ | y²⁺ | #2 |
| 1 | 114.16719 | 57.58729 | L | | | 11 |
| 2 | 242.29810 | 121.65275 | Q | 1162.19921 | 581.60330 | 10 |
| 3 | 371.41370 | 186.21055 | E | 1034.06830 | 517.53785 | 9 |
| 4 | 499.58820 | 250.29780 | K | 904.95270 | 452.98005 | 8 |
| 5 | 600.69330 | 300.85035 | T | 776.77820 | 388.89280 | 7 |
| 6 | 729.80890 | 365.40815 | E | 675.67310 | 338.34025 | 6 |
| 7 | 857.93981 | 429.47360 | Q | 546.55750 | 273.78245 | 5 |
| 8 | 987.05541 | 494.03140 | E | 418.42659 | 209.71699 | 4 |
| 9 | 1058.13411 | 529.57075 | A | 289.31099 | 145.15919 | 3 |
| 10 | 1129.21281 | 565.11010 | A | 218.23229 | 109.61984 | 2 |
| 11 | | | Q | 147.15359 | 74.08049 | 1 |

### Example 19

### GAD1 Protein Breakdown Products

FIG. 24 is an MS/MS spectrum displaying the fragment ions for the glutamate decarboxylase 1 (GAD1) peptide HPRFFNQLSTGLDIIGLAG (SEQ ID NO:410) identified in human TBI CSF ultrafiltrate. The identified b- and y-type ions for this peptide are shown in Table 38, below. Peptide ions in italics and underline were found in MS/MS spectra. The presence of PFs of GAD1 in TBI CSF indicates that GAD1 protein and its higher molecular weight breakdown products can serve as biomarkers for central nervous system injury, and to monitor astroglial damage.

TABLE 38
Additional Data for FIG. 24.

| | | | | SEQ ID | | | | |
|---|---|---|---|---|---|---|---|---|
| #1 | b⁺ | b²⁺ | b³⁺ | NO: 411 | y⁺ | y²⁺ | y³⁺ | #2 |
| 1 | 138.14879 | 69.57809 | 46.72119 | H | | | | 19 |
| 2 | 235.26559 | 118.13649 | 79.09346 | P | 1920.22109 | 960.61424 | 640.74529 | 18 |
| 3 | 391.45359 | 196.23049 | 131.15613 | R | 1823.10429 | 912.05584 | 608.37302 | 17 |
| 4 | 538.63079 | 269.81909 | 180.21519 | F | 1666.91629 | 833.96184 | 556.31036 | 16 |
| 5 | 685.80799 | 343.40769 | 229.27426 | F | 1519.73909 | 760.37324 | 507.25129 | 15 |
| 6 | 799.91189 | 400.45964 | 267.30889 | N | 1372.56189 | 686.78464 | 458.19222 | 14 |
| 7 | 928.04280 | 464.52510 | 310.01920 | Q | 1258.45799 | 629.73269 | 420.15759 | 13 |
| 8 | 1041.20260 | 521.10500 | 347.73913 | L | 1130.32708 | 565.66724 | 377.44729 | 12 |
| 9 | 1128.28060 | 564.64400 | 376.76513 | S | 1017.16728 | 509.08734 | 339.72735 | 11 |
| 10 | 1229.38570 | 615.19655 | 410.46683 | T | 930.08928 | 465.54834 | 310.70135 | 10 |
| 11 | 1286.43740 | 643.72240 | 429.48406 | G | 828.98418 | 414.99579 | 276.99965 | 9 |
| 12 | 1399.59720 | 700.30230 | 467.20400 | L | 771.93248 | 386.46994 | 257.98242 | 8 |
| 13 | 1514.68570 | 757.84655 | 505.56683 | D | 658.77268 | 329.89004 | 220.26249 | 7 |
| 14 | 1627.84550 | 814.42645 | 543.28676 | I | 543.68418 | 272.34579 | 181.89965 | 6 |
| 15 | 1741.00530 | 871.00635 | 581.00670 | I | 430.52438 | 215.76589 | 144.17972 | 5 |
| 16 | 1798.05700 | 899.53220 | 600.02393 | G | 317.36458 | 159.18599 | 106.45979 | 4 |
| 17 | 1911.21680 | 956.11210 | 637.74386 | L | 260.31288 | 130.66014 | 87.44255 | 3 |
| 18 | 1982.29550 | 991.65145 | 661.43676 | A | 147.15308 | 74.08024 | 49.72262 | 2 |
| 19 | | | | G | 76.07438 | 38.54089 | 26.02972 | 1 |

### Example 20

### Synapsin Protein Breakdown Products and Peptide Fragments

FIG. 25 is an MS/MS spectrum for the Synapsin-1 (SYN1) peptide QDEVKAETIRS (SEQ ID NO:412) that can be identified in human TBI CSF ultrafiltrate, displaying the fragment ions for this peptide. Table 39, below shows the identified b- and y-type ions for this peptide. Peptide ions in italics and underline were found in MS/MS spectra. The presence of PFs of SYN1 in TBI CSF implies that SYN1 protein and its higher molecular weight breakdown products are suitable for use as biomarkers according to the invention.

TABLE 39
MS/MS Data for FIG. 25.

| | | | SEQ ID | | | |
|---|---|---|---|---|---|---|
| #1 | b⁺ | b²⁺ | NO: 413 | y⁺ | y²⁺ | #2 |
| 1 | 29.13830 | 65.07285 | Q | | | 11 |
| 2 | 244.22680 | 122.61710 | D | 1148.25928 | 574.63334 | 10 |
| 3 | 373.34240 | 187.17490 | E | 1033.17078 | 517.08909 | 9 |
| 4 | 472.47520 | 236.74130 | V | 904.05518 | 452.53129 | 8 |
| 5 | 600.64970 | 300.82855 | K | 804.92238 | 402.96489 | 7 |
| 6 | 671.72840 | 336.36790 | A | 676.74788 | 338.87764 | 6 |
| 7 | 800.84400 | 400.92570 | E | 605.66918 | 303.33829 | 5 |
| 8 | 901.94910 | 451.47825 | T | 476.55358 | 238.78049 | 4 |
| 9 | 1015.10890 | 508.05815 | I | 375.44848 | 188.22794 | 3 |
| 10 | 1171.29690 | 586.15215 | R | 262.28868 | 131.64804 | 2 |
| 11 | | | S | 106.10068 | 53.55404 | 1 |

FIG. 26 is an MS/MS spectrum for the Synapsin-2 (SYN2) peptide SQSLTNAFSFSESSFFRS (SEQ ID NO:414) identified in human TBI CSF ultrafiltrate, displaying the fragment ions for this peptide. The identified b- and y-type ions for this peptide are shown in Table 40, below. Peptide ions in italics and underline were found in MS/MS spectra. The presence of the breakdown products of SYN2 in TBI CSF indicates that SYN2 protein and its higher molecular weight breakdown products are suitable according to the invention for use as biomarkers for central nervous system injury.

TABLE 40
MS/MS Data for FIG. 26.

| | | | | SEQ ID | | | | |
|---|---|---|---|---|---|---|---|---|
| #1 | b⁺ | b²⁺ | b³⁺ | NO: 415 | y⁺ | y²⁺ | y³⁺ | #2 |
| 1 | 88.08539 | 44.54639 | 30.03339 | S | | | | 18 |
| 2 | 216.21630 | 108.61185 | 72.74370 | Q | 1943.08149 | 972.04444 | 648.36542 | 17 |
| 3 | 303.29430 | 152.15085 | 101.76970 | S | 1814.95058 | 907.97899 | 605.65512 | 16 |
| 4 | 416.45410 | 208.73075 | 139.48963 | L | 1727.87258 | 864.43999 | 576.62912 | 15 |
| 5 | 517.55920 | 259.28330 | 173.19133 | T | 1614.71278 | 807.86009 | 538.90919 | 14 |
| 6 | 631.66310 | 316.33525 | 211.22596 | N | 1513.60768 | 757.30754 | 505.20749 | 13 |
| 7 | 702.74180 | 351.87460 | 234.91886 | A | 1399.50378 | 700.25559 | 467.17285 | 12 |
| 8 | 849.91900 | 425.46320 | 283.97793 | F | 1328.42508 | 664.71624 | 443.47995 | 11 |
| 9 | 936.99700 | 469.00220 | 313.00393 | S | 1181.24788 | 591.12764 | 394.42089 | 10 |
| 10 | 1084.17420 | 542.59080 | 362.06300 | F | 1094.16988 | 547.58864 | 365.39489 | 9 |
| 11 | 1171.25220 | 586.12980 | 391.08900 | 5 | 946.99268 | 474.00004 | 316.33582 | 8 |
| 12 | 1300.36780 | 650.68760 | 434.12753 | E | 859.91468 | 430.46104 | 287.30982 | 7 |
| 13 | 1387.44580 | 694.22660 | 463.15353 | S | 730.79908 | 365.90324 | 244.27129 | 6 |
| 14 | 1474.52380 | 737.76560 | 492.17953 | S | 643.72108 | 322.36424 | 215.24529 | 5 |
| 15 | 1621.70100 | 811.35420 | 541.23860 | F | 556.64308 | 278.82524 | 186.21929 | 4 |
| 16 | 1768.87820 | 884.94280 | 590.29766 | F | 409.46588 | 205.23664 | 137.16022 | 3 |
| 17 | 1925.06620 | 963.03680 | 642.36033 | R | 262.28868 | 131.64804 | 88.10115 | 2 |
| 18 | | | | S | 106.10068 | 53.55404 | 36.03849 | |

FIG. 27 is an MS/MS spectra for the Synapsin-3 (SYN3) PF DWSKYFHGKKVNGEIEIRV (SEQ ID NO:416) identified in human TBI CSF ultrafiltrate, displaying the fragment ions for this peptide. The identified b- and y-type ions are shown in Table 41, below. Peptide ions in italics and underline were found in MS/MS spectra. The presence of the PFs of SYN3 in TBI CSF indicates that SYN3 protein and its higher molecular weight breakdown products are present and in higher in biofluids (CSF) from TBI subjects than in controls. This example shows that human biofluid-based monitoring of SYN1, SYN2 and SYN3 PFs can be used to monitor presynaptic terminal injury.

TABLE 41
MS/MS Data for FIG. 27.

| | | | | SEQ ID | | | | |
|---|---|---|---|---|---|---|---|---|
| #1 | b⁺ | b²⁺ | b³⁺ | NO: 417 | y⁺ | y²⁺ | y³⁺ | #2 |
| 1 | 116.09589 | 58.55164 | 39.37023 | D | | | | 19 |
| 2 | 302.30999 | 151.65869 | 101.44159 | W | 2191.54519 | 1096.27629 | 731.18666 | 18 |
| 3 | 389.38799 | 195.19769 | 130.46759 | S | 2005.33109 | 1003.16924 | 669.11529 | 17 |
| 4 | 517.56249 | 259.28494 | 173.19243 | K | 1918.25309 | 959.63024 | 640.08929 | 16 |
| 5 | 680.73910 | 340.87325 | 227.58463 | Y | 1790.07859 | 895.54299 | 597.36446 | 15 |
| 6 | 827.91630 | 414.46185 | 276.64370 | F | 1626.90198 | 813.95469 | 542.97225 | 14 |
| 7 | 965.05770 | 483.03255 | 322.35750 | H | 1479.72478 | 740.36609 | 493.91319 | 13 |
| 8 | 1022.10940 | 511.55840 | 341.37473 | G | 1342.58338 | 671.79539 | 448.19939 | 12 |
| 9 | 1150.28390 | 575.64565 | 384.09956 | K | 1285.53168 | 643.26954 | 429.18215 | 11 |
| 10 | 1278.45840 | 639.73290 | 426.82440 | K | 1157.35718 | 579.18229 | 386.45732 | 10 |
| 11 | 1377.59120 | 689.29930 | 459.86866 | V | 1029.18268 | 515.09504 | 343.73249 | 9 |
| 12 | 1491.69510 | 746.35125 | 497.90330 | N | 930.04988 | 465.52864 | 310.68822 | 8 |
| 13 | 1548.74680 | 774.87710 | 516.92053 | G | 815.94598 | 408.47669 | 272.65359 | 7 |
| 14 | 1677.86240 | 839.43490 | 559.95906 | E | 758.89428 | 379.95084 | 253.63635 | 6 |
| 15 | 1791.02220 | 896.01480 | 597.67900 | I | 629.77868 | 315.39304 | 210.59782 | 5 |
| 16 | 1920.13780 | 960.57260 | 640.71753 | E | 516.61888 | 258.81314 | 172.87789 | 4 |
| 17 | 2033.29760 | 1017.15250 | 678.43746 | I | 387.50328 | 194.25534 | 129.83935 | 3 |
| 18 | 2189.48560 | 1095.24650 | 730.50013 | R | 274.34348 | 137.67544 | 92.11942 | 2 |
| 19 | | | | V | 118.15548 | 59.58144 | 40.05675 | 1 |

### Example 21

### Striatin Protein Breakdown Products and Peptide Fragments

FIG. 28 is an MS/MS spectrum for the Striatin peptide AGLTVANEADSLTYD (SEQ ID NO:418) identified in human TBI CSF ultrafiltrate, displaying the fragment ions for this peptide. The identified b- and y-type ions for this peptide shown from the database search are shown in Table 42, below. Peptide ions in italics and underline were found in MS/MS spectra. The presence of proteolytic breakdown products (peptides) of Striatin in TBI CSF indicates that Striatin protein and its higher molecular weight breakdown products are present and are higher in biofluids (CSF) from TBI subjects than in controls. Since striatin is specifically expressed in striatum, this example shows that human biofluid-based monitoring of Striatin PBPs or PFs can be used to monitor striatum injury.

TABLE 42
MS/MS Data for FIG. 28.

| | | | SEQ ID | | | |
|---|---|---|---|---|---|---|
| #1 | b⁺ | b²⁺ | NO: 419 | y⁺ | y²⁺ | #2 |
| 1 | 72.08609 | 36.54674 | A | | | 15 |
| 2 | 129.13779 | 65.07259 | G | 1469.54549 | 735.27644 | 14 |
| 3 | 242.29759 | 121.65249 | L | 1412.49379 | 706.75059 | 13 |
| 4 | 343.40269 | 172.20504 | T | 1299.33399 | 650.17069 | 12 |
| 5 | 442.53549 | 221.77144 | V | 1198.22889 | 599.61814 | 11 |
| 6 | 513.61419 | 257.31079 | A | 1099.09609 | 550.05174 | 10 |
| 7 | 627.71809 | 314.36274 | N | 1028.01739 | 514.51239 | 9 |
| 8 | 756.83369 | 378.92054 | E | 913.91349 | 457.46044 | 8 |
| 9 | 827.91239 | 414.45989 | A | 784.79789 | 392.90264 | 7 |
| 10 | 943.00089 | 472.00414 | D | 713.71919 | 357.36329 | 6 |
| 11 | 1030.07889 | 515.54314 | S | 598.63069 | 299.81904 | 5 |
| 12 | 1143.23869 | 572.12304 | L | 511.55269 | 256.28004 | 4 |
| 13 | 1244.34379 | 622.67559 | T | 398.39289 | 199.70014 | 3 |
| 14 | 1407.52040 | 704.26390 | Y | 297.28779 | 149.14759 | 2 |
| 15 | | | D | 134.11118 | 67.55929 | 1 |

### Example 22

### GAP43 Protein Breakdown Products and Peptide Fragments

FIG. 29 is an MS/MS spectrum for the GAP43 peptide AETESATKASTDNSPSSKAEDA (SEQ ID NO:420) identified in human TBI CSF ultrafiltrate, displaying the fragment ions for this peptide. The identified b- and y-type ions for this peptide shown from the database search are shown in Table 43, below. Peptide ions in italics and underline were found in MS/MS spectra. The presence of PFs of GAP43 in TBI CSF indicates that GAP43 protein and its higher molecular weight breakdown products are present and in higher in biofluids (CSF) from TBI subjects than in controls. Since GAP43 is specifically expressed in neurite growth cones, this example shows that human biofluid-based monitoring of Striatin PBPs or PFs can be used to monitor neurite growth cones.

TABLE 43
MS/MS Data for FIG. 29.

| | | | | SEQ ID | | | | |
|---|---|---|---|---|---|---|---|---|
| #1 | b⁺ | b²⁺ | b³⁺ | NO: 421 | y⁺ | y²⁺ | y³⁺ | #2 |
| 1 | 72.08609 | 36.54674 | 24.70029 | A | | | | 22 |
| 2 | 201.20169 | 101.10454 | 67.73883 | E | 2127.13628 | 1064.07184 | 709.71702 | 21 |
| 3 | 302.30679 | 151.65709 | 101.44053 | T | 1998.02068 | 999.51404 | 666.67849 | 20 |
| 4 | 431.42239 | 216.21489 | 144.47906 | E | 1896.91558 | 948.96149 | 632.97679 | 19 |
| 5 | 518.50039 | 259.75389 | 173.50506 | S | 1767.79998 | 884.40369 | 589.93825 | 18 |
| 6 | 589.57909 | 295.29324 | 197.19796 | A | 1680.72198 | 840.86469 | 560.91225 | 17 |
| 7 | 690.68419 | 345.84579 | 230.89966 | T | 1609.64328 | 805.32534 | 537.21935 | 16 |
| 8 | 818.85869 | 409.93304 | 273.62449 | K | 1508.53818 | 754.77279 | 503.51765 | 15 |
| 9 | 889.93739 | 445.47239 | 297.31739 | A | 1380.36368 | 690.68554 | 460.79282 | 14 |
| 10 | 977.01539 | 489.01139 | 326.34339 | S | 1309.28498 | 655.14619 | 437.09992 | 13 |
| 11 | 1078.12049 | 539.56394 | 360.04509 | T | 1222.20698 | 611.60719 | 408.07392 | 12 |
| 12 | 1193.20899 | 597.10819 | 398.40793 | D | 1121.10188 | 561.05464 | 374.37222 | 11 |
| 13 | 1307.31289 | 654.16014 | 436.44256 | N | 1006.01338 | 503.51039 | 336.00939 | 10 |
| 14 | 1394.39089 | 697.69914 | 465.46856 | S | 891.90948 | 446.45844 | 297.97475 | 9 |
| 15 | 1491.50769 | 746.25754 | 497.84083 | P | 804.83148 | 402.91944 | 268.94875 | 8 |
| 16 | 1578.58569 | 789.79654 | 526.86683 | S | 707.71468 | 354.36104 | 236.57649 | 7 |
| 17 | 1665.66369 | 833.33554 | 555.89283 | S | 620.63668 | 310.82204 | 207.55049 | 6 |
| 18 | 1793.83819 | 897.42279 | 598.61766 | K | 533.55868 | 267.28304 | 178.52449 | 5 |
| 19 | 1864.91689 | 932.96214 | 622.31056 | A | 405.38418 | 203.19579 | 135.79965 | 4 |
| 20 | 1994.03249 | 997.51994 | 665.34909 | E | 334.30548 | 167.65644 | 112.10675 | 3 |
| 21 | 2109.12099 | 1055.06419 | 703.71193 | D | 205.18988 | 103.09864 | 69.06822 | 2 |
| 22 | | | | A | 90.10138 | 45.55439 | 30.70539 | 1 |

### Example 23

### Microtubule-associated Protein 6 Protein Breakdown Products and Peptide Fragments

FIG. 30A is an MS/MS spectrum for the MAP6 PF TKYSEATEHPGAPPQPPPPQQ (aa 31-51; SEQ ID NO:422) identified in human TBI CSF ultrafiltrate, displaying the fragment ions for this peptide. The identified b- and y-type ions for this peptide shown from the database search are provided in Table 44, below. Peptide ions in italics and underline are found in MS/MS spectra.

TABLE 44
MS/MS Data for FIG. 30A.

| | | | | SEQ ID | | | | |
|---|---|---|---|---|---|---|---|---|
| #1 | b⁺ | b²⁺ | b³⁺ | NO: 423 | y⁺ | y²⁺ | y³⁺ | #2 |
| 1 | 102.05 | 51.53 | 34.69 | T | | | | 21 |
| 2 | 230.15 | 115.58 | 77.39 | K | 2156.04 | 1078.52 | 719.35 | 20 |
| 3 | 393.21 | 197.11 | 131.74 | Y | 2027.95 | 1014.48 | 676.65 | 19 |
| 4 | 480.25 | 240.63 | 160.75 | S | 1864.88 | 932.94 | 622.3 | 18 |
| 5 | 609.29 | 305.15 | 203.77 | E | 1777.85 | 889.43 | 593.29 | 17 |
| 6 | 680.32 | 340.67 | 227.45 | A | 1648.81 | 824.91 | 550.27 | 16 |
| 7 | 781.37 | 391.19 | 261.13 | T | 1577.77 | 789.39 | 526.6 | 15 |
| 8 | 910.42 | 455.71 | 304.14 | E | 1476.72 | 738.87 | 492.91 | 14 |
| 9 | 1047.47 | 524.24 | 349.83 | H | 1347.68 | 674.34 | 449.9 | 13 |
| 10 | 1144.53 | 572.77 | 382.18 | P | 1210.62 | 605.81 | 404.21 | 12 |
| 11 | 1201.55 | 601.28 | 401.19 | G | 1113.57 | 557.29 | 371.86 | 11 |
| 12 | 1272.59 | 636.8 | 424.87 | A | 1056.55 | 528.78 | 352.85 | 10 |
| 13 | 1369.64 | 685.32 | 457.22 | P | 985.51 | 493.26 | 329.17 | 9 |
| 14 | 1466.69 | 733.85 | 489.57 | P | 888.46 | 444.73 | 296.82 | 8 |
| 15 | 1594.75 | 797.88 | 532.25 | Q | 791.4 | 396.21 | 264.47 | 7 |
| 16 | 1691.8 | 846.4 | 564.61 | P | 663.35 | 332.18 | 221.79 | 6 |
| 17 | 1788.86 | 894.93 | 596.96 | P | 566.29 | 283.65 | 189.44 | 5 |
| 18 | 1885.91 | 943.46 | 629.31 | P | 469.24 | 235.12 | 157.09 | 4 |
| 19 | 1982.96 | 991.98 | 661.66 | P | 372.19 | 186.6 | 124.73 | 3 |
| 20 | 2111.02 | 1056.01 | 704.34 | Q | 275.14 | 138.07 | 92.38 | 2 |
| 21 | | | | Q | 147.08 | 74.04 | 49.7 | 1 |

FIG. 30B is an MS/MS spectrum for the MAP6 PF QLPTVSPLPRVMIPTAPHTEYIESS (aa 788-812; SEQ ID NO:424) identified in human TBI CSF ultrafiltrate, displaying the fragment ions for this peptide. The identified b- and y-type ions for this peptide shown from the database search are provided in Table 45, below. Peptide ions in italics and underline are found in MS/MS spectra.

TABLE 45
MS/MS Data for FIG. 30B.

| | | | | SEQ ID | | | | |
|---|---|---|---|---|---|---|---|---|
| #1 | b⁺ | b²⁺ | b³⁺ | NO: 425 | y⁺ | y²⁺ | y³⁺ | #2 |
| 1 | 129.07 | 65.04 | 43.69 | Q | | | | 25 |
| 2 | 242.15 | 121.58 | 81.39 | L | 2715.35 | 1358.18 | 905.79 | 24 |
| 3 | 339.2 | 170.1 | 113.74 | P | 2602.26 | 1301.63 | 868.09 | 23 |
| 4 | 440.25 | 220.63 | 147.42 | T | 2505.21 | 1253.11 | 835.74 | 22 |
| 5 | 539.32 | 270.16 | 180.44 | V | 2404.16 | 1202.58 | 802.06 | 21 |
| 6 | 626.35 | 313.68 | 209.46 | S | 2305.09 | 1153.05 | 769.04 | 20 |
| 7 | 723.4 | 362.21 | 241.81 | P | 2218.06 | 1109.53 | 740.03 | 19 |
| 8 | 836.49 | 418.75 | 279.5 | L | 2121.01 | 1061.01 | 707.67 | 18 |
| 9 | 933.54 | 467.27 | 311.85 | P | 2007.92 | 1004.47 | 669.98 | 17 |
| 10 | 1089.64 | 545.32 | 363.89 | R | 1910.87 | 955.94 | 637.63 | 16 |
| 11 | 1188.71 | 594.86 | 396.91 | V | 1754.77 | 877.89 | 585.6 | 15 |
| 12 | 1319.75 | 660.38 | 440.59 | M | 1655.7 | 828.35 | 552.57 | 14 |
| 13 | 1432.83 | 716.92 | 478.28 | I | 1524.66 | 762.83 | 508.89 | 13 |
| 14 | 1529.89 | 765.45 | 510.63 | P | 1411.58 | 706.29 | 471.2 | 12 |
| 15 | 1630.93 | 815.97 | 544.32 | T | 1314.52 | 657.77 | 438.85 | 11 |
| 16 | 1701.97 | 851.49 | 568 | A | 1213.48 | 607.24 | 405.16 | 10 |
| 17 | 1799.02 | 900.02 | 600.35 | P | 1142.44 | 571.72 | 381.48 | 9 |
| 18 | 1936.08 | 968.55 | 646.03 | H | 1045.39 | 523.2 | 349.13 | 8 |
| 19 | 2117.1 | 1059.05 | 706.37 | T (p) | 908.33 | 454.67 | 303.45 | 7 |
| 20 | 2246.14 | 1123.57 | 749.38 | E | 727.31 | 364.16 | 243.11 | 6 |
| 21 | 2409.2 | 1205.11 | 803.74 | Y | 598.27 | 299.64 | 200.1 | 5 |
| 22 | 2522.29 | 1261.65 | 841.43 | I | 435.21 | 218.11 | 145.74 | 4 |
| 23 | 2651.33 | 1326.17 | 884.45 | E | 322.12 | 161.57 | 108.05 | 3 |
| 24 | 2738.36 | 1369.68 | 913.46 | S | 193.08 | 97.04 | 65.03 | 2 |
| 25 | | | | S | 106.05 | 53.53 | 36.02 | 1 |

The presence of PFs of MAP6 in TBI CSF indicates that MAP6 protein and its higher molecular weight breakdown products are present and higher in biofluids (CSF) from TBI subjects than in controls. This example shows that human biofluid-based monitoring of MAP6 PFs can be used to monitor dendritic injury.

The sequence of microtubule-associated protein 6 (human) (Q96JE9-1) is:

SEQ ID NO: 426
MAWPCITRACCIARFWNQLDKADIAVPLVFTKYSEATEHPGAPPQPPPPQ
QQAQPALAPPSARAVAIETQPAQGELDAVARATGPAPGPTGEREPAAGPG
RSGPGPGLGSGSTSGPADSVMRQDYRAWKVQRPEPSCRPRSEYQPSDAPF
ERETQYQKDFRAWPLPRRGDHPWIPKPVQISAASQASAPILGAPKRRPQS
QERWPVQAAAEAREQEAAPGGAGGLAAGKASGADERDTRRKAGPAWIVRR
AEGLGHEQTPLPAAQAQVQATGPEAGRGRAAADALNRQIREEVASAVSSS
YRNEFRAWTDIKPVKPIKAKPQYKPPDDKMVHETSYSAQFKGEASKPTTA
DNKVIDRRRIRSLYSEPFKEPPKVEKPSVQSSKPKKTSASHKPTRKAKDK
QAVSGQAAKKKSAEGPSTTKPDDKEQSKEMNNKLAEAKESLAQPVSDSSK
TQGPVATEPDKDQGSVVPGLLKGQGPMVQEPLKKQGSVVPGPPKDLGPMI
PLPVKDQDHTVPEPLKNESPVISAPVKDQGPSVPVPPKNQSPMVPAKVKD
QGSVVPESLKDQGPRIPEPVKNQAPMVPAPVKDEGPMVSASVKDQGPMVS
APVKDQGPIVPAPVKGEGPIVPAPVKDEGPMVSAPIKDQDPMVPEHPKDE
SAMATAPIKNQGSMVSEPVKNQGLVVSGPVKDQDVVVPEHAKVHDSAVVA
PVKNQGPVVPESVKNQDPILPVLVKDQGPTVLQPPKNQGRIVPEPLKNQV
PIVPVPLKDQDPLVPVPAKDQGPAVPEPLKTQGPRDPQLPTVSPLPRVMI
PTAPHTEYIESSP.

Regions in bold are MAP6 PFs found in human TBI CSF ultrafiltrate samples.

### Example 24

### Nesprin-1 Protein Breakdown Products and Peptide Fragments

FIG. 31 is an MS/MS spectrum for the Nesprin-1 PF HSAKEELHR (SEQ ID NO:427) identified in human TBI CSF ultrafiltrate, displaying the fragment ions for this peptide. The identified b- and y-type ions for this peptide shown from the database search are provided in Table 46, below. Peptide ions in italics and underline are found in MS/MS spectra. The presence of PFs of Nesprin-1 in TBI CSF indicates that Nesprin-1 protein and its higher molecular weight breakdown products are present and in higher in biofluids (CSF) from TBI subjects than in controls. This example shows that human biofluid-based monitoring of Nesprin-1 PFs can be used to monitor neuronal nuclear damage

TABLE 46
Additional Data for FIG. 31.

| | | | | SEQ ID | | | | |
|---|---|---|---|---|---|---|---|---|
| #1 | b⁺ | b²⁺ | b³⁺ | NO: 428 | y⁺ | y²⁺ | y³⁺ | #2 |
| 1 | 138.14879 | 69.57809 | 46.72119 | H | | | | 9 |
| 2 | 225.22679 | 113.11709 | 75.74719 | S | 970.07428 | 485.54084 | 324.02969 | 8 |
| 3 | 296.30549 | 148.65644 | 99.44009 | A | 882.99628 | 442.00184 | 295.00369 | 7 |
| 4 | 424.47999 | 212.74369 | 142.16493 | K | 811.91758 | 406.46249 | 271.31079 | 6 |
| 5 | 553.59559 | 277.30149 | 185.20346 | E | 683.74308 | 342.37524 | 228.58595 | 5 |
| 6 | 682.71119 | 341.85929 | 228.24199 | E | 554.62748 | 277.81744 | 185.54742 | 4 |
| 7 | 795.87099 | 398.43919 | 265.96193 | L | 425.51188 | 213.25964 | 142.50889 | 3 |
| 8 | 933.01239 | 467.00989 | 311.67573 | H | 312.35208 | 156.67974 | 104.78895 | 2 |
| 9 | | | | R | 175.21068 | 88.10904 | 59.07515 | 1 |

### Example 25

### Neurexin-3 Protein Breakdown Products and Peptide Fragments

FIG. 32 is an MS/MS spectrum for the Neurexin-3 PF IVLLPLPTAY (SEQ ID NO:429) identified in human TBI CSF ultrafiltrate, displaying the fragment ions for this peptide. The identified b- and y-type ions for this peptide shown from the database search are shown in Table 47, below. Peptide ions in italics and underline are found in MS/MS spectra. The presence of PFs of Neurexin-3 in TBI CSF indicates that Neurexin-3 protein and its higher molecular weight breakdown products are present and in higher in biofluids (CSF) from TBI subjects than in controls. This example shows that human biofluid-based monitoring of Neurexin-3 PFs can be used to monitor presynaptic terminal injury.

TABLE 47
MS/MS Data for FIG. 32.

| | | | | SEQ ID | | | | |
|---|---|---|---|---|---|---|---|---|
| #1 | b⁺ | b²⁺ | b³⁺ | NO: 430 | y⁺ | y²⁺ | y³⁺ | #2 |
| 1 | 114.16719 | 57.58729 | 38.72733 | I | | | | 10 |
| 2 | 213.29999 | 107.15369 | 71.77159 | V | 987.22889 | 494.11814 | 329.74789 | 9 |
| 3 | 326.45979 | 163.73359 | 109.49153 | L | 888.09609 | 444.55174 | 296.70362 | 8 |
| 4 | 439.61959 | 220.31349 | 147.21146 | L | 774.93629 | 387.97184 | 258.98369 | 7 |
| 5 | 536.73639 | 268.87189 | 179.58373 | P | 661.77649 | 331.39194 | 221.26376 | 6 |
| 6 | 649.89619 | 325.45179 | 217.30366 | L | 564.65969 | 282.83354 | 188.89149 | 5 |
| 7 | 747.01299 | 374.01019 | 249.67593 | P | 451.49989 | 226.25364 | 151.17156 | 4 |
| 8 | 848.11809 | 424.56274 | 283.37763 | T | 354.38309 | 177.69524 | 118.79929 | 3 |
| 9 | 919.19679 | 460. 10209 | 307.07053 | A | 253.27799 | 127.14269 | 85.09759 | 2 |
| 10 | | | | Y | 182.19929 | 91.60334 | 61.40469 | 1 |

### Example 26

### Chondroitin Sulfate Proteoglycan 4 Protein Breakdown Products and Peptide Fragments

FIG. 33 is an MS/MS spectrum for the Chondroitin sulfate proteoglycan 4 (CSPG4) PF YEHEMPPEPFWEAHD (SEQ ID NO:431) identified in human TBI CSF ultrafiltrate, displaying the fragment ions for this peptide. The identified b- and y-type ions for this peptide shown from the database search are provided in Table 48, below. Peptide ions in italics and underline are found in MS/MS spectra. The presence of PFs of CSPG4 in TBI CSF indicates that CSPG4 protein and its higher molecular weight breakdown products are present and in higher in biofluids (CSF) from TBI subjects than in controls. This example shows that human biofluid-based monitoring of CSPG4 PFs can be used to monitor brain extracellular matrix damage.

TABLE 48
MS/MS Data for FIG. 33.

| | | | | SEQ ID | | | | |
|---|---|---|---|---|---|---|---|---|
| #1 | b⁺ | b²⁺ | b³⁺ | NO: 432 | y⁺ | y²⁺ | y³⁺ | #2 |
| 1 | 164.18400 | 82.59570 | 55.39960 | Y | | | | 15 |
| 2 | 293.29960 | 147.15350 | 98.43813 | E | 1751.87618 | 876.44179 | 584.63032 | 14 |
| 3 | 430.44100 | 215.72420 | 144.15193 | H | 1622.76058 | 811.88399 | 541.59179 | 13 |
| 4 | 559.55660 | 280.28200 | 187.19046 | E | 1485.61918 | 743.31329 | 495.87799 | 12 |
| 5 | 690.75600 | 345.88170 | 230.92360 | M | 1356.50358 | 678.75549 | 452.83945 | 11 |
| 6 | 787.87280 | 394.44010 | 263.29586 | P | 1225.30418 | 613.15579 | 409.10632 | 10 |
| 7 | 884.98960 | 442.99850 | 295.66813 | P | 1128.18738 | 564.59739 | 376.73405 | 9 |
| 8 | 1014.10520 | 507.55630 | 338.70666 | E | 1031.07058 | 516.03899 | 344.36179 | 8 |
| 9 | 1111.22200 | 556.11470 | 371.07893 | P | 901.95498 | 451.48119 | 301.32325 | 7 |
| 10 | 1258.39920 | 629.70330 | 420.13800 | F | 804.83818 | 402.92279 | 268.95099 | 6 |
| 11 | 1444.61330 | 722.81035 | 482.20936 | W | 657.66098 | 329.33419 | 219.89192 | 5 |
| 12 | 1573.72890 | 787.36815 | 525.24790 | E | 471.44688 | 236.22714 | 157.82055 | 4 |
| 13 | 1644.80760 | 822.90750 | 548.94080 | A | 342.33128 | 171.66934 | 114.78202 | 3 |
| 14 | 1781.94900 | 891.47820 | 594.65460 | H | 271.25258 | 136.12999 | 91.08912 | 2 |
| 15 | | | | D | 134.11118 | 67.55929 | 45.37532 | 1 |

### Example 27

Complment protein Breakdown Products and Peptide Fragments. As shown in Table 48A, Complement protein Clqb, C3, C5, Cls, and CR1 peptides were identified in only human CSF samples, not control CSF samples.

TABLE 48A
Complement protein C1q, C3, C5, C1s and CR1 peptides identified in human CSF
samples
Complement C1q subcomponent subunit B D6R934

| | | | | |
|---|---|---|---|---|
| Peptide: | HGEFGEKGDPGIPG | Microglia activation | SEQ ID NO: | 701 |

Complement C3 P01024

| | | | | |
|---|---|---|---|---|
| Peptide: | HWESASLL | Microglia activation | SEQ ID NO: | 702 |
| Peptide: | VKVFSLAVNLIAI | Microglia activation | SEQ ID NO: | 703 |

Complement receptor type 1 CR1 E9PDY4

| | | | | |
|---|---|---|---|---|
| Peptide: | KTPEQFPFAS | Microglia activation | SEQ ID NO: | 704 |

Complement C5 P01031

| | | | | |
|---|---|---|---|---|
| Peptide: | VTcTNAELVKGRQ | Microglia activation | SEQ ID NO: | 705 |

Complement C1s P09871

| | | | | |
|---|---|---|---|---|
| Peptide: | IISGDTEEGRLcGQ | Microglia activation | SEQ ID NO: | 706 |
| | RSSNNPHSPIVE | | | |

### Example 27

### Summary Information

Table 49, below, is a spreadsheet showing additional representative PFs from brain proteins uniquely identified from human CSF ultrafiltrate samples. Table 50, below, shows combined evidence of PFs from brain proteins (peptidome) found in brain ultrafiltrate in the mouse model of TBI and/or in CSF samples from human TBI subjects. This summarizes the results showing that human biofluid-based monitoring of additional brain protein derived PFs can be used to monitor central nervous system injury such as TBI.

TABLE 49
Representative Peptide Fragments Uniquely Identified from Human CSF Ultrafiltrate
Samples.

| | | Protein | | | | | | | | |
|---|---|---|---|---|---|---|---|---|---|---|
| | Human | length | | | | | | | | SEQ |
| Protein (accession | Gene | (Peptide | | | | | MH+ | ΔM | m/z | ID |
| number) | Name | location) | Sequence | ΔCn | XCorr | Charge | (Da) | (ppm) | (Da) | NO |
| Brevican Core | BCAN | 911 | YENWNPGQPDSYFLSGE | 1.224 | 2.03 | 3 | 3474.66 | −20.18 | 1158.89 | 433 |
| Protein | | (765-793) | NcVVmVWHDQGQ¹ | | | | | | | |
| (Q96GW7) | | | | | | | | | | |
| Calmodulin- | CAMSAP1 | 1613 | NSLTRVDGQPRGAAIA | 1.008 | 2.54 | 3 | 1910.64 | 256.93 | 637.55 | 434 |
| regulated Spectrin- | | (456-473) | WP | | | | | | | |
| associated Protein | | | | | | | | | | |
| 1 (Q5T5Y3-3) | | | | | | | | | | |
| Calmodulin- | CAMSAP3 | 1249 | ASSPAATNSEVKmTSFA | 0.185 | 2.26 | 3 | 2257.03 | −223.61 | 753.01 | 435 |
| regulated Spectrin- | | (551-572) | ERKK² | | | | | | | |
| associated Protein | | | | | | | | | | |
| 3 (Q9P1Y5) | | | | | | | | | | |
| Calmodulin- | CAMK2B | 666 | FGFAGTPGYLSPEVLRK | 0.238 | 2.64 | 3 | 2482.71 | −635.85 | 828.24 | 436 |
| dependent Protein | | (172-194) | EAYGKP | | | | | | | |
| Kinase IIB | | | | | | | | | | |
| (Q13554) | | | | | | | | | | |
| Chondroitin | CSPG4 | 2322 | PEPFWEAHDT | 0.325 | 2.31 | 2 | 1229.24 | −44.05 | 615.12 | 437 |
| Sulfate | | (1664- | | | | | | | | |
| Proteoglycan | | 1673) | | | | | | | | |
| (Q6UVK1) | | | | | | | | | | |
| Disks Large | DLG4 | 721 | LGFSIAGGTDN | 0.161 | 2.51 | 3 | 1052.26 | 127.36 | 351.43 | 438 |
| (postsynaptic | | (72-82) | | | | | | | | |
| density protein95, | | | | | | | | | | |
| PSD-95) | | | | | | | | | | |
| (P78352-3) | | | | | | | | | | |
| Fructose- | ALDOC | 364 | EEFIKRAEVNGLAA | 1.183 | 2.27 | 2 | 1547.30 | −293.99 | 37.02 | 439 |
| bisphosphate | | (325-339) | | | | | | | | |
| aldolase C | | | | | | | | | | |
| (P09972) | | | | | | | | | | |
| Glial fibrillary | GFAP | 432 | DGEVIKESKQEHKDVM | 0.154 | 2.67 | 2 | 1873.82 | 383.80 | 937.41 | 440 |
| acidic protein | | (417-432) | | | | | | | | |
| (P14136) | | | | | | | | | | |
| Huntingtin | HTT | 3142 | YITAAcEmVAEmVESLQ | 0.170 | 2.05 | 3 | 2163.96 | −241.51 | 721.99 | 441 |
| (P42858) | | (2355- | SV³ | | | | | | | |
| | | 2373) | | | | | | | | |
| Macrophage | MRC1 | 1456 | GQASLEcLRMGSSLVSIE | 0.000 | 1.78 | 3 | 3462.39 | −144.43 | 1154.80 | 442 |
| mannose receptor | | (1257- | SAAESSFLSYRVEP⁴ | | | | | | | |
| 1; MRC1, CD208 | | 1288) | | | | | | | | |
| (P22897) | | | | | | | | | | |
| Microtubule- | MAP2 | 1823 | TYEQALAKDLSI | 0.293 | 2.08 | 2 | 1352.50 | −20.10 | 676.75 | 443 |
| associated protein | | (991-1002) | | | | | | | | |
| 2; isoform 3 | | | | | | | | | | |
| (P11137) | | | | | | | | | | |
| Microtubule- | MAP6 | 813 | TKYSEATEHPGAPPQPP | 0.145 | 3.65 | 3 | 2258.48 | 12.70 | 753.50 | 444 |
| associated protein | | (31-51) | PPQQ | | | | | | | |
| 6 (Q96JE9) | | | | | | | | | | |
| Golli-MBP, | MBPA1 | 304 | NAWQDAHPADPGSRPH | 0.235 | 2.16 | 3 | 1982.92 | −117.80 | 661.65 | 445 |
| isoform 3; myelin | | (75-92) | LI | | | | | | | |
| A1; HOG7 | | | | | | | | | | |
| (P02686-1) | | | | | | | | | | |
| Myelin basic | MBP | 197 | IVTPRTPPPSQG | 0.238 | 2.25 | 2 | 1250.09 | −277.39 | 625.55 | 446 |
| protein, isoform 4 | | (120-131) | | | | | | | | |
| (P02686-3) | | | | | | | | | | |
| Myelin expression | MYEF2 | 600 | LGSAmIGGFAGRIGSSN | 0.206 | 2.04 | 3 | 2803.90 | 260.88 | 935.31 | 447 |
| factor 2 | | (435-465) | MGPVGSGISGGmGS⁵ | | | | | | | |
| (Q9P2K5) | | | | | | | | | | |
| Myelin | MYT1 | 1121 | IKQLNQEIRDLNESNSEm | 0.150 | 2.04 | 3 | 2378.06 | −219.52 | 793.36 | 448 |
| transcription factor | | (1004- | EA⁶ | | | | | | | |
| 1 (Q01538) | | 1023) | | | | | | | | |
| Neurocan core | NCAN | 1321 | GHLTSVHSPEEHSFINSF | 1.187 | 3.07 | 3 | 3632.11 | 44.04 | 1211.38 | 449 |
| protein; CSPG3 | | (1121- | GHENTWIGLNDRIV | | | | | | | |
| (O14594) | | 1152) | | | | | | | | |
| Neurogranin | NRGN | 78 | GPGGPGGAGVARGGA | 0.594 | 2.77 | 2 | 1407.27 | 546.06 | 704.14 | 450 |
| (Q92686) | | (57-75) | GGGP | | | | | | | |
| Nesprin-1 | SYNE1 | 8797 | HSAKEELHR | 0.123 | 2.15 | 3 | 1106.67 | −494.70 | 369.56 | 451 |
| (Q8NF91) | | (2856- | | | | | | | | |
| | | 2865) | | | | | | | | |
| Neurexin-1 alpha | NRXN1 | 1547 | VDFFAIEmLDGHLYLLL | 0.136 | 2.17 | 3 | 3841.27 | 164.67 | 1281.09 | 452 |
| (Q9ULB1-3) | | (578-610) | DmGSGTIKIKALLKKVN⁷ | | | | | | | |
| Neurofilament | NEFM | 877 | KEEEPEAEEEEVAAKKS | 0.162 | 2.85 | 3 | 2128.81 | −222.20 | 710.28 | 453 |
| medium | | (495-513) | PV | | | | | | | |
| polypeptide | | | | | | | | | | |
| (E7ESP9) | | | | | | | | | | |
| Neurofilament | NEFL | 543 | AMQDTINKLENELRTTK | 0.123 | 2.57 | 3 | 2424.94 | 73.55 | 808.99 | 454 |
| light polypeptide | | (346-366) | SEMA | | | | | | | |
| (P07196) | | | | | | | | | | |
| Spectrin alpha | SPTAN1 | 2477 | KMKGLNGKVSDLEKA | 0.110 | 2.05 | 2 | 1619.36 | 259.70 | 810.18 | 455 |
| chain, | | (1955- | | | | | | | | |
| nonerythrocytic 1 | | 1969) | | | | | | | | |
| (A6NG51; Q13813-2) | | | | | | | | | | |
| Spectrin, beta, | SPTBN1 | 2155 | AQQYYFDAAEAEAWM | 0.154 | 2.19 | 3 | 3610.48 | −126.72 | 1204.17 | 456 |
| nonerythrocytic 1 | | (1581- | SEQELYmMSEEKAKD⁸ | | | | | | | |
| (Q01082) | | 1610) | | | | | | | | |
| Spectrin beta | SPTBN2 | 2390 | GQYSDINNRWDLPDSD | 0.136 | 2.22 | 2 | 2082.75 | 288.29 | 1041.88 | 457 |
| nonerythrocytic 2 | | (17-33) | W | | | | | | | |
| (O15020) | | | | | | | | | | |
| Spectrin, beta, | SPTBN4 | 2564 | EHAEIARWGQTL | 1.177 | 2.03 | 2 | 1410.77 | −558.29 | 705.89 | 458 |
| nonerythrocytic 4 | | (2527- | | | | | | | | |
| (Q9H254) | | 2539) | | | | | | | | |
| Striatin | STRN | 780 | DPYDSYDPSVLRGPL | 0.165 | 2.57 | 3 | 1695.15 | 182.57 | 565.72 | 459 |
| (O43815) | | (551-565) | | | | | | | | |
| Synapsin I | SYN1 | 705 | QASQAGPVPRTGPPTTQ | 0.181 | 2.04 | 3 | 2074.61 | −329.93 | 692.21 | 460 |
| (P17600-2) | | (603-622) | QPR | | | | | | | |
| Synapsin III | SYN3 | 580 | GRDYIIEVmDSSmPLIGE | 0.176 | 2.25 | 3 | 3796.92 | −94.71 | 1266.31 | 461 |
| (P07437) | | (359-391) | HVEEDRQLmADLVVS⁹ | | | | | | | |
| Synaptojanin-1 | SYNJ1 | 1350 | SHSLPSEASSQPQQEQPS | 1.103 | 3.36 | 2 | 1982.57 | 275.16 | 991.79 | 462 |
| (C9JFZ1) | | (1332- | G | | | | | | | |
| | | 1350) | | | | | | | | |
| Tubulin beta 5 | TUBB | 444 | GSQQYRALTVPELTQQV | 0.117 | 2.32 | 3 | 2898.60 | −250.19 | 966.87 | 463 |
| (P07437) | | (277-301) | FDAKNMMAA | | | | | | | |
| Vimentin | VIM | 466 | TLLIKTVETRDGQVIN | 1.000 | 2.11 | 2 | 1798.96 | −591.29 | 899.98 | 464 |
| (P08670) | | (441-456) | | | | | | | | |

¹Modification: C19(Carbamidomethyl); M22(Oxidation).
²Modification: M13(Oxidation).
³Modification: C6(Carbamidomethyl); M8(Oxidation); M12(Oxidation)
⁴Modification: C7(Carbamidomethyl)
⁵Modification: M5(Oxidation); M29(Oxidation)
⁶Modification: M18(Oxidation)
⁷Modification: M8(Oxidation); M19(Oxidation)
⁸Modification: M22(Oxidation)
⁹Modification: M9(Oxidation); M13(Oxidation); M27(Oxidation)

TABLE 50
Summary of Peptide Fragments from Brain Proteins Found in Brain Ultrafiltrate
Samples (Mouse Model of TBI and/or CSF Samples from Human TBI Subjects).

| | | in vivo Mouse | |
|---|---|---|---|
| | Gene name (based | Brain Lysate | Human CSF |
| Protein Name | on human) | CCI | TBI |
| Amyloid beta A4 precursor protein-binding family B | APBB1 | + | + |
| member 1-interacting protein | | | |
| Brain soluble acidic protein 1 | BASP1 | | |
| Brevican core protein (CSPG7) | BCAN | + | + |
| Calmodulin regulated spectrin-associated protein 1 | CAMSAP1 | | + |
| Calmodulin regulated spectrin-associated protein 2 | CAMSAP2 | + | |
| Calmodulin regulated spectrin-associated protein 3 | CAMSAP3 | + | + |
| Chondroitin sulfate proteoglycan 4 | CSPG4 | + | + |
| Cortexin-1 | | + | |
| Creatine kinase B-type | CKB | + | |
| Disks large homolog 4, PSD95 | DLG4 | | + |
| Disks large homolog 2, PSD98 | DLG2 | + | |
| Fructose-bisphosphate aldolase C | ALDOC | | + |
| Glial fibrillary acidic protein | GFAP | | + |
| Glutamate decarboxylase 1 | GAD1 | + | |
| Glutamate decarboxylase 2 | GAD2 | + | |
| Huntingtin | HTT | + | + |
| Macrophage mannose receptor 1 (CD208) | MRC1 | + | + |
| Microtubule-associated protein 1A | MAP1A | + | |
| Microtubule-associated protein 1B | MAP1B | + | |
| Microtubule-associated protein 2 | MAP2 | + | + |
| Microtubule-associated protein 6 | MAP6 | + | + |
| Microtubule-associated serine/threonine-protein | MAST1 | | + |
| kinase 1 | | | |
| Myelin basic protein, isoform 4 (21.5K) | MBP (4) | + | + |
| Golli-MBP (Myelin basic protein-A1) | MBP (A1) | + | + |
| Myelin transcription factor 1 | MYT1 | | + |
| Myelin expression factor 2 | MYEF2 | | + |
| Myelin regulatory factor | MYRF | + | |
| Nesprin-1 | SYNE1 | | + |
| Neurexin-1 | NRXN1 | + | + |
| Neurexin-3 | NRXN3 | + | |
| Neurocan | NCAN | + | + |
| Neurochondrin | NCDN | + | |
| Neurofascin | NFASC | + | |
| Neurofilament heavy polypeptide | NEFH | + | |
| Neurofilament light polypeptide | NEFL | + | + |
| Neurofilament medium polypeptide | NEFM | | + |
| Neurogranin | NRGN | + | + |
| Secretogranin-2 | SCG2 | + | |
| Striatin | STRN | | + |
| Synapsin I | SYN1 | | + |
| Synapsin II | SYN2 | + | |
| Synapsin III | SYN3 | | + |
| Synaptotagmin-2 | SYT2 | + | |
| Synaptojanin-1 | SYNJ1 | | + |
| Synuclein, beta | SNCB | | + |
| Tau | MAPT | + | + |
| Tubulin beta-4A chain | TUBB4A | + | |
| Tubulin beta-4B chain | TUBB4B | + | |
| Tubulin beta 5 | TBB5 | | + |
| Vimentin | VIM | + | + |

Additional key novel TBI PBP biomarkers identified were derived from Synapsin-I, II, III (SYN1, SYN2, SYN3), Cortexin-1,2,3 (CTXN1, CTXN2, CTXN3), Striatin (STRN), NRGN, Golli-MBP1, Tau-758, VIM, Brain acidic soluble protein (BASP1, BASP2 (GAP33)), Nesprin-1, Glutamate Decarboxylase-1, 2 (GAD1, GAD2), Neurexin-1, 2, 3 (NRXN1, NRXN2, NRXN3) Calmodulin-binding spectrin associated proteins-1, 2, 3 (CAMSAP1, 2, 3), and Chondroitin sulfate proteoglycans (CSPG4, Neurocan (CSPG3, brevican), and Neurochondrin. These proteins are listed in Table 48, with supporting data in Table 15. This example shows that human biofluid-based monitoring of additional these brain protein derived PBPs and/or PFs can be used to monitor brain injury such as TBI.

### Example 28

### Diagnosis of Trauma to the Central Nervous System

For diagnosis, prognosis or monitoring of trauma to the central nervous system the biofluid levels of protein, PBPs and PFs, or a battery of proteins, PBPs and/or PFs are measured. An initial subject fluid biological sample (such as blood, serum, plasma or CSF) is obtained within 24 or 72 hours after traumatic injury or suspected traumatic injury to the CNS (such as TBI), preferably within 24 hours after traumatic injury. The sample is subjected to ultrafiltration with a molecular cutoff of 10,000 Da, using a centrifugation-based ultrafiltration cell. The retentate is subjected to protein analysis. The filtrate is subjected to testing for PFs using an antibody-based immunoassay according to procedures well-known in the art, using antibodies that specifically recognize AEPRQEFEVMEDHAGTYGLG (SEQ ID NO:465), NVKMALDIEIAT (SEQ ID NO:466), DGEVIKES (SEQ ID NO:467), and GRTQDENPVVHFFKNIVTPRTPPPSQGKGRGLSLSRF (SEQ ID NO:468). The signal indicating the amount of the peptide is compared to the signal from an equivalent control sample from a control, uninjured subject. An amount of one or more PFs that is two times the control amount, indicates an injury. Sample interpretations of results are shown in Table 51.

TABLE 51
Exemplary Sample Results.

| PF or PBP | Result | Specific Indication or Diagnosis |
|---|---|---|
| Synapsin I PBP | 4-fold higher in | Traumatic injury to the CNS with |
| | levels in CNS | severe synaptic damage |
| | injury subject's | |
| | blood sample than | |
| | average levels in | |
| | normal control | |
| | subjects | |
| GFAP (DGEVIKES; SEQ ID | 2-3 fold higher in | Traumatic injury to the CNS with |
| NO: 469) | levels in CNS | moderate astroglia cell injury |
| | injury subject's | |
| | blood sample than | |
| | average levels in | |
| | normal control | |
| | subjects | |
| MAP6 | 4-fold higher in | Traumatic injury to the CNS with |
| (TKYSEATEHPGAPPQPPPPQQ; | levels in CNS | severe dendritic damage |
| SEQ ID NO: 470) | injury subject's | |
| | blood sample than | |
| | average levels in | |
| | normal control | |
| | subjects | |
| Striatin PBP | 3-fold higher in | Traumatic injury to the brain with |
| | levels in CNS | moderate damage to the striatum |
| | injury subject's | |
| | blood sample than | |
| | average levels in | |
| | normal control | |
| | subjects | |
| Cortexin-1 PBP | 6-fold higher in | Traumatic injury to the brain with |
| | levels in CNS | very severe damage to the cortex |
| | injury subject's | |
| | blood sample than | |
| | average levels in | |
| | normal control | |
| | subjects | |
| A Panel of the Synapsin I-PBP, | with the above | Traumatic injury to the CNS |
| GFAP PF and MAP6 PF (above) | indicated results | (brain/spinal cord) with severe |
| | | synaptic and dendritic damage, |
| | | but moderate astroglia cell injury |
| A Panel of Striatin PBP and | with the above | Traumatic injury to the brain with |
| Cortexin-1 PBP | indicated results | very severe cortex injury, but |
| | | moderate striatum injury |

In order to determine the prognosis of the subject above, the following further tests should be performed on samples collected from the subject at the following times: 24 hours, 48 hours and 72 hours post injury. If the 72-hour results are less than ⅓ of the levels for the 24-hour results, the prognosis is good to excellent; if the 72-hour biomarker test levels are about the same as or higher than the levels seen in the sample taken at 24 hours, the prognosis is poor.

### Example 29

For novel Golli-MBP protein, FIG. 33A is example of mouse mass culture clones against Golli-MBP N-terminal peptide region HAGKRELNAEKAST with ELISA test against this peptide region.

FIG. 33B and the right column of FIG. 33A showed the same mass culture clones against Golli-MBP N-terminal peptide region HAGKRELNAEKAST has showing strong detection of Golli-MBP (33 kDa) against human lysate. These data support that base don our FP peptides from Golli-MBP, one can derive useful antibody that can detect full length Golli-MBP protein in human brain tissue sample

### Example 29

### Interpretation of Results

By comparing the signals yielded for specific proteins, PBPs and/or PFs to available standards (such as cranial/spinal computer tomography (CTO or Magnetic resonance imaging (MRI) detectable abnormality or Glasgow coma scale score, or Glasgow outcome scale score), their cutoff values can be assigned. Such cutoff values are compared to control samples or to a prepared chart of levels to determine the severity of the injury, or the prognosis of the subject, or monitoring of the patient injury progression or recovery. For example, higher biofluid levels of one or more protein, PBP or PF indicates the subject is more severely injured, more likely to develop post-trauma complications, or to prone to have poor patient outcome. For example, for blood levels of a protein, PBP or PF (e.g. as derived from synapsin) usually would have levels in control subjects of less than 10 pg/mL, while mild to moderate CNS injured subjects generally are expected to have a level between 10-50 pg/mL, and more severe CNS injury subjects generally are expected to have a level above 50 pg/mL

In another example, at least two measurements of these proteins, PBPs, and PFs as biomarkers are assayed in an initial and at least one subsequent sample. For example, first measurement within 24 hours of the incident, and a second or additional measurement after the first 24 hours. The values of these biomarker levels over time provide the ability to monitor the progression of the traumatic injury or the recovery of the CNS from the initial traumatic injury. For example, a CNS trauma subject that is on course for good recovery with no complications would have biomarker levels in the second or additional measurements that are lower than the biomarker levels of the same biomarker(s) at a prior measurement. On the other hand, a subject who has biomarker(s) levels in the second or additional measurements that are higher than the biomarker levels of the same biomarker(s) at a prior measurement could indicate there is a deterioration or evolution of the injury condition, development secondary injury or post-trauma neurodegeneration development. For this later group, once identified, more aggressive medical monitoring and/or medical intervention then can be administrated.

### REFERENCES

References listed below and throughout the specification are hereby incorporated by reference in their entirety.
1. U.S. Pat. No. 7,291,710 to Hayes, et al.
2. U.S. Pat. No. 7,396,654 to Hayes, et al.
3. U.S. Pat. No. 7,456,027 to Hayes, et al.
4. U.S. Pat. No. 7,611,858 to Svetlov, et al.
5. International Patent Publication No. PCT/US2015/024880 to Wang.
6. Wang, Trends Neurosci. 23:59, 2000.
7. Yang et al., PLOS ONE 5, e15878, 2010.
8. Yang et al., J. Cerebral Blood Flow Metab. 34:1444-1452, 2014.
9. Wang et al., Expert Rev. Molec. Diagnostics E-pub PMID: 29338452, 2018

1. A method of diagnosing trauma to the central nervous system in a subject in need thereof, comprising:
testing a first fluid biological sample obtained from the subject for the level of at least two proteins, or their protein breakdown products (about 85%, or less, the size of the intact proteins and greater than 10 kDa) and lower molecular weight peptide fragments (ranging from 500 Da to 10 kDa) selected from the group consisting of
(a) Neurogranin-protein breakdown products, or peptide fragment
(b) Tau-758 (Tau-G) isoform;
(c) Tau-441 (Tau-F)N- or C-terminal peptide fragment
(d) Synapsin (Synapsin I, Synapsin II, Synapsin III);
(e) Vimentin;
(f) GFAP-C- and N-terminal peptide fragments
(g) Golli-Myelin Basic Protein (MBP) (without or with classic MBP);
(h) MAP6;
(i) Complement protein (C1q (a, b, c components), C3, C5, C1s, CR1, CR2, C1QRF) wherein levels of the at least two proteins or their protein breakdown products, or peptide fragments that are at least two-fold higher in the fluid biological sample from the subject than the levels of the at least two proteins or protein breakdown products in a fluid biological sample from an uninjured subject indicate the presence of a central nervous system injury.

2. The method of claim 1 wherein the at least two peptide fragments are selected from the group consisting of:
Phospho-Neurogranin peptide (position 16-64) ILDIPLDDPGANAAAAKIQAS(p)*FRGHMARKKIKSGERGRKGPGPGGPGGA (*(p)=phospho-Serine)(SEQ ID NO: 482),
Neurogranin peptide (position 16-64) ILDIPLDDPGANAAAAKIQASFRGHMARKKIKSGERGRKGPGPGGPGGA (SEQ ID NO: 483),
Neurogranin peptide (position 57-75) GPGGPGGAGVARGGAGGGP (SEQ ID NO: 450),
Golli-MBP N-terminal peptide HGSKYLATASTMD (SEQ ID NO: 494),
Golli-MBP internal peptide NAWQDAHPADPGSRPHLIRLFSRDAPGREDNTFKDRPSESDE (SEQ ID NO: 499)
Tau-G (P10636-9; 776 aa)-specific peptide (internal) [411-457]
SPKHPTPGSSDPLIQPSSPAVCPEPPSSPKYVSSVTSRTGSSGAKEM (SEQ ID NO: 477)
Tau-441 (P10636-8, 441 aa)N-terminal peptide [2-21] AEPRQEFEVMEDHAGTYGLG_(SEQ ID NO: 471)
Tau-441 (P10636-8, 441 aa)C-terminal peptide [421-438] SPQLATLADEVSASLAK (SEQ ID NO: 474);
GFAP N-terminal peptide [12-33] RSYVSSGEMMVGGLAPGRRLGP (SEQ ID NO: 502),
GFAP C-terminal peptide [388-400] QIRETSLDTKSVSE (SEQ ID NO: 81),
GFAP C-terminal peptide [417-423] DGEVIKES (SEQ ID NO:506);
Vimentin N-terminal peptide [1-75] MSTRSVSSSS YRRMFGGPGT ASRPSSSRSY VTTSTRTYSL GSALRPSTSR SLYASSPGGV YATRSSAVRL RSSVP (SEQ ID NO: 492),
Vimentin C-terminal peptide [400-464] YRKLLEGEESR ISLPLPTFSS
LNLRETNLES LPLVDTHSKR TLLIKTVETR DGQVINETSQ HHDD (SEQ ID NO: 490),
Classic MBP peptide [isoform−1; 115-125] KNIVTPRTPPP (SEQ ID NO: 195),
Classic MBP peptide, [isoform−5; 105-140] GRTQDENPVVHFFKNIVTPRTPPPSQGKGRGLSLSRF (SEQ ID NO: 162; SEQ ID NO: 347)
Classic MBP peptide [isoform-1; 107-116] TQDENPVVHF (SEQ ID NO: 322)

3. A method of diagnosing trauma to the central nervous system in a subject in need thereof, comprising:
testing a first fluid biological sample obtained from the subject for the level of a Phospho-Neurogranin peptide (position 16-64) ILDIPLDDPGANAAAAKIQAS(p)FRGHMARKKIKSGERGRKGPGPGGPGGA (SEQ ID NO: 482); and/or a Neurogranin peptide (position 16-64) ILDIPLDDPGANAAAAKIQASFRGHMARKKIKSGERGRKGPGPGGPGGA (SEQ ID NO: 483)
wherein levels of the Phospho-Neurogranin peptide and/or Neurogranin peptide that are at least two-fold higher in the fluid biological sample from the subject than the levels in a fluid biological sample from an uninjured subject indicate the presence of a central nervous system injury.

4. The method of claim 1, claim 2 or claim 3 wherein the first fluid biological sample is obtained from the subject within 24 hours of the trauma to the central nervous system.

5. The method of claim 1, claim 2 or claim 3 wherein the first fluid biological sample is obtained from the subject within 3 days of the trauma to the central nervous system.

6. The method of claim 1, claim 2 or claim 3 wherein one or more additional fluid biological samples are obtained from the subject at subsequent times to the first fluid biological sample.

7. The method of claim 1, claim 2 or claim 3 wherein the testing comprises subjecting the fluid biological samples are subjected to ultrafiltration using a ultrafiltration membrane filter with a molecular weight cutoff of about 10,000 Da to separate an ultrafiltrate fraction and then subjecting the ultrafiltrate fraction to assay for proteins, protein breakdown products or peptide fragments.

8. The method of claim 1, claim 2 or claim 3 wherein an increasing level of the at least two proteins, protein breakdown products, or peptide fragments in fluid biological samples taken at subsequent times indicates worsening of the severity of the central nervous system injury.

9. The method of claim 1, claim 2 or claim 3 wherein a decreasing level of the at least two proteins, protein breakdown products, or peptide fragments in fluid biological samples taken at subsequent times indicates improvement in the central nervous system injury.

10. The method of claim 1, claim 2 or claim 3 wherein an unchanging level of the at least two proteins, protein breakdown products, or peptide fragments in fluid biological samples taken at subsequent times indicates a leveling of the severity of the central nervous system injury.

11. The method of claim 1, claim 2 or claim 3 wherein the testing will additionally examine the anatomical location of trauma to the central nervous system in a subject in need thereof, comprising additional testing a fluid biological sample obtained from the subject for the presence of any combination of:
(a) one or more cortexin proteins, protein breakdown products, or peptide fragments, the presence of which above control levels identifies the cortex as the anatomical location;
(b) one or more myelin basic protein proteins, protein breakdown products, or peptide fragments, the presence of which above control levels identifies the white matter as the anatomical location; and
(c) one or more striatin proteins, protein breakdown products, or peptide fragments, the presence of which above control levels identifies the striatum as the anatomical location.

12. The method of claim 1, claim 2 or claim 3 wherein the testing will additionally examine cell types injured in trauma to the central nervous system in a subject in need thereof, comprising testing a fluid biological sample obtained from the subject for the presence of any combination of:
(a) one or more protein, or protein breakdown product of brain acidic soluble protein−1, glutamate decarboxylase 1, glutamate decarboxylase 2, neurochondrin or any combination thereof, the presence of which above control levels identifies the cell type as neurons;
(b) one or more protein, or protein breakdown product of GFAP or Vimentin, the presence of which above control levels identifies the cell type as astroglia; or
(c) one or more protein, or protein breakdown product of myelin basic protein 5 or Golli-myelin basic protein, the presence of which above control levels identifies the cell type as oligodendrocytes.

13. The method of claim 1, claim 2 or claim 3 wherein the testing will additionally examine the subcellular location of injury to the central nervous system after trauma in a subject in need thereof, comprising testing a fluid biological sample obtained from the subject for the presence of any combination of:
(a) one or more protein, or protein breakdown product of neurexin-1, neurexin-2, neurexin-3, synapsin-I, synapsin-II, synapsin-III or any combination thereof, the presence of which above control levels identifies the subcellular location as the presynaptic terminal;
(b) one or more protein, or protein breakdown product of neurogranin, the presence of which above control levels identifies the subcellular location as the post-synaptic terminal;
(c) one or more protein, or protein breakdown product of brain acidic soluble protein 2, growth associated protein 43 or a combination thereof, the presence of which above control levels identifies the subcellular location as the growth cone;
(d) one or more protein, or protein breakdown product of nesprin-1, the presence of which above control levels identifies the subcellular location as the neuronal nucleus;
(e) one or more protein, or protein breakdown product of Calmodulin regulated spectrin-associated protein 1, Calmodulin regulated spectrin-associated protein 2, Calmodulin regulated spectrin-associated protein 3, or any combination thereof, the presence of which above control levels identifies the subcellular location as the cortical cytoskeleton and axon;
(f) one or more protein, or protein breakdown product of microtubule associated protein 6, the presence of which above control levels identifies the subcellular location as dendrites; or
(g) one or more protein, or protein breakdown product of chondroitin sulfate proteoglycan 4, neurocan, brevican or any combination thereof, the presence of which above control levels identifies the subcellular location as the extracellular matrix.

14. A method of diagnosing the severity of trauma to the central nervous system in a subject in need thereof, comprising the steps of:
(a) testing a first fluid biological sample obtained from the subject up to 3 days after central nervous system injury for the levels of one or more proteins, protein breakdown products, and peptide fragments selected from claim 1, claim 2 or claim 3
(b) testing a second subsequent fluid biological sample obtained from the subject subsequent to the first fluid biological sample for the levels of the same one or more proteins, protein breakdown products, and peptide fragments as step (a);
(c) optionally testing further subsequent fluid biological samples for the levels of the same one or more proteins, protein breakdown products, and peptide fragments as step (a);
(d) comparing the levels of the one or more proteins, protein breakdown products, and peptide fragments in the fluid biological samples to a control sample from an uninjured subject and to each other; and
(e) when the levels of peptide breakdown products in the fluid biological samples increase in subsequent samples, diagnosing a severe central nervous system injury.

15. A method of distinguishing severe trauma to the central nervous system with pathoanatomical lesions detectable by CT, MRI, or both, from less severe central nervous system trauma with no detectable pathoanatomical lesions in a subject in need thereof, comprising:
(a) testing at least one first fluid biological sample obtained from the subject within 24 hours after central nervous system injury for the levels of one or more peptide fragments of a protein selected from claim 1, claim 2 or claim 3;
(b) testing a second subsequent fluid biological sample obtained from the subject about 2 days to about 6 months subsequent to the first fluid biological sample for the levels of the same one or more peptide fragments as step (a);
(c) comparing the levels of the same one or more peptide fragments in the first and second fluid biological samples to a control sample from an uninjured subject and to each other; and
(d) when the levels of the same one or more peptide fragments in the first fluid biological sample are above those in the control sample but decrease in the second fluid biological samples, diagnosing an acute central nervous system injury; and when the levels of the same one or more peptide fragments in the first fluid biological samples are above those in the control sample and increase or remain constant in subsequent samples, diagnosing a chronic central nervous system injury.

16. The method of claim 1, claim 2 or claim 3 wherein the trauma is cortical impact, closed head injury, blast overpressure induced brain injury, concussion or spinal cord injury.

17. The method of claim 1, claim 2 or claim 3 wherein the fluid biological sample is cerebrospinal fluid, blood, plasma, serum, saliva, urine, wound fluid, or biopsy, necropsy or autopsy samples of brain tissue, spinal tissue, retinal tissue, and/or nerves.

18. A diagnostic kit comprising:
(a) detection agents for antibody, aptamer or mass spectrometry detection methods for detection of one or more peptide fragments selected from the group consisting of
Phospho-Neurogranin peptide (position 16-64) ILDIPLDDPGANAAAAKIQAS (p)FRGHMARKKIKS GERGRKGPGPGGPGGA (*(p) phospho-Serine) (SEQ ID NO: 482),
Neurogranin peptide (position 16-64) ILDIPLDDPGANAAAAKIQASFRGHMARKKIKSGERGRKGPGPGGPGGA (SEQ ID NO: 483),
Neurogranin peptide (position 57-75) GPGGPGGAGVARGGAGGGP (SEQ ID NO: 450),
Golli-MBP N-terminal peptide HGSKYLATASTMD (SEQ ID NO: 494),
Golli-MBP internal peptide NAWQDAHPADPGSRPHLIRLFSRDAPGREDNTFKDRPSESDE (SEQ ID NO: 499)
Tau-G (P10636-9; 776 aa)-specific peptide (internal) [411-457]
SPKHPTPGSSDPLIQPSSPAVCPEPPSSPKYVSSVTSRTGSSGAKEM (SEQ ID NO: 477)
Tau-441 (P10636-8, 441 aa)N-terminal peptide [2-21] AEPRQEFEVMEDHAGTYGLG (SEQ ID NO: 471)
Tau-441 (P10636-8, 441 aa)C-terminal peptide [421-438] SPQLATLADEVSASLAK (SEQ ID NO: 474);
GFAP N-terminal peptide [12-33] RSYVSSGEMMVGGLAPGRRLGP (SEQ ID NO: 502),
GFAP C-terminal peptide [388-400] QIRETSLDTKSVSE (SEQ ID NO: 81),
GFAP C-terminal peptide [417-423] DGEVIKES (SEQ ID NO:506);
Vimentin N-terminal peptide [1-75] MSTRSVSSSS YRRMFGGPGT ASRPSSSRSY VTTSTRTYSL GSALRPSTSR SLYASSPGGV YATRSSAVRL RSSVP_(SEQ ID NO: 492),
Vimentin C-terminal peptide [400-464] YRKLLEGEESR ISLPLPTFSS LNLRETNLES LPLVDTHSKR TLLIKTVETR DGQVINETSQ HHDD (SEQ ID NO: 490),
Classic MBP Peptide [isoform−1; 115-125] KNIVTPRTPPP (SEQ ID NO: 195),
Classic MBP peptide, [isoform−5; 105-140] GRTQDENPVVHFFKNIVTPRTPPPSQGKGRGLSLSRF (SEQ ID NO: 162; SEQ ID NO: 347)
Classic MBP peptide [isoform-1; 107-116] TQDENPVVHF (SEQ ID NO: 322)
(b) an analyte protein, protein breakdown product, or peptide fragment to serve as internal standard and/or positive control; and
(c) a signal generation coupling component.
